(19) United States
(12) Patent Application Publication
(10) Pub. No.: US 2022/0213514 A1
(43) Pub. Date: Jul. 7, 2022

(54) GENETICALLY MODIFIED MICROORGANISM FOR PRODUCING 3-HYDROXYHEXANEDIOIC ACID, (E)-HEX-2-ENEDIOIC ACID AND/OR HEXANEDIOIC ACID, AND PRODUCTION METHOD FOR SAID CHEMICALS

(71) Applicant: TORAY INDUSTRIES, INC., Tokyo (JP)

(72) Inventors: Kyohei ISOBE, Kamakura-shi (JP); Kenji KAWAMURA, Kamakura-shi (JP); Katsushige YAMADA, Kamakura-shi (JP)

(73) Assignee: TORAY INDUSTRIES, INC., Tokyo (JP)

(21) Appl. No.: 17/609,964

(22) PCT Filed: May 8, 2020
(86) PCT No.: PCT/JP2020/018663

§ 371 Date: Nov. 9, 2021

(30) Foreign Application Priority Data
May 10, 2019 (JP) 2019-089770

(51) Int. Cl. C12P 7/42; C12P 7/44; C12N 15/70

(52) U.S. Cl. CPC C12P 7/42; C12N 15/70; C12P 7/44

(57) ABSTRACT

Disclosed is a genetically modified microorganism that produces 3-hydroxyadipic acid, α-hydromuconic acid. or adipic acid in high yield. A nucleic acid encoding any one of the polypeptides described in (a) to (c) below is introduced or the expression of the polypeptide is enhanced and the function of pyruvate kinase is impaired in the genetically modified microorganism: (a) a polypeptide composed of an amino acid sequence represented by any one of SEQ ID NOs: 1 to 7; (b) a polypeptide having the same amino acid sequence as any one of those amino acid sequences, except that one or several amino acids are substituted, deleted, inserted, andor added, and having an enzymatic activity that catalyzes a reaction to reduce 3-oxoadipyl-CoA to 3-hydroxyadipyl-CoA; (c) a polypeptide composed of an amino acid sequence with a sequence identity of not less than 70% to any one of those amino acid sequences and having an enzymatic activity that catalyzes a reaction to reduce 3-oxoadipyl-CoA to 3-hydroxyadipyl-CoA.

### TECHNICAL FIELD

The present invention relates to a genetically modified microorganism in which a nucleic acid encoding a polypeptide involved in the production of a substance of interest is introduced or the expression of the polypeptide is enhanced, and to a method of producing the substance by using the microorganism.

### BACKGROUND ART

3-Hydroxyadipic acid (IUPAC name: 3-hydroxyhexariediiiic acid), α-hydromuconic acid (IUPAC name: (E)-hex-2-enedioic acid), and adipic acid (IUPAC name: hexanedioic acid) are dicarboxylic acids containing six carbon atoms. These dicarboxylic acids can be polymerized with a polyhydric alcohol or a polyfunctional amine, to be used as raw materials for the production of polyesters or polyamides, respectively. Additionally, these dicarboxylic acids can be used alone after ammonia addition at a terminal position in these chemicals to form lactams as raw materials for the production of polyamides.

The following documents related to the production of 3-hydroxyadipic acid or α-hydromuconic acid using a microorganism are known.

Patent Document 1 describes a method of producing 1,3-butadiene by using a microorganism in which a relevant metabolic pathway is modified, wherein 3-hydroxyadipic acid (3-hydroxyadipate) is described to be a metabolic intermediate in the metabolic pathway for biosynthesis of 1,3-butadiene from acetyl-CoA and succinyl-CoA.

Patent Document 2 describes a method of producing muconic acid by using a microorganism in which a relevant metabolic pathway is modified, wherein α-hydromuconic acid (2,3-dehydroadipate) is described to be a metabolic intermediate in the metabolic pathway for biosynthesis of trans,trans-muconic acid from acetyl-CoA and succinyl-CoA.

Patent Documents 3 and 4 describe a method of producing adipic acid and hexamethylene diamine (HMDA) by using a non-natural microorganism, wherein the biosynthetic pathways for these substances are described to share a common reaction to synthesize 3-oxoadipyi-CoA from acetyl-CoA and succinyi-CoA but diverge after the synthesis of 3-oxoadipyl-CoA. Furthermore, Patent Document 3 describes the pyruvate kinase gene as a candidate gene that is additionally deleted from the metabolic pathway to improve the HMDA formation coupled with proliferation for the IIMDA production, but a potential relationship between pyruvate kinase deficiency and increased adipic acid production is not mentioned in this document.

Additionally, all the biosynthetic pathways mentioned in Patent Documents 1 to 4 are described to share a common enzymatic reaction that reduces 3-oxoadipyl-CoA to 3-hydroxyadipyl-CoA.

Patent Documents 5 and 6 describe methods of producing 3-hydroxyadipic acid and α-hydromuconic acid by using a microorganism of the genus Serratia , respectively. The patent documents disclose that the efficiency of producing 3-hydroxyadipic acid and α-hydromuconic acid can be increased particularly by enhancing the activity of an acyl transferase that catalyzes a reaction to produce 3-oxoadipyl-CoA from acetyl-CoA and succinyl-CoA, but these documents have no description related to pyruvate kinase.

Moreover, a method of modifying a microorganism based on an in silky) analysis is disclosed in Patent Document 7, in which the production of succinic acid is increased by deleting genes encoding pyruvate kinase and a phosphotransferase system enzyme in Escherichia coli (E. coli), pykF, pykA, and ptsG, and culturing the resulting E. coli bacteria under anaerobic conditions.

### PRIOR ART DOCUMENTS

### Patent Documents

Patent Document 1: JP 2013-535203 A
Patent Document 2: US 20110124911 A1
Patent Document 3: JP 2015-146810 A
Patent Document 4: JP 2011-515111 A
Patent Document 5: WO 2017209102
Patent Document 6: WO 2017209103
Patent Document 7: JP 2008-527991 A

### SUMMARY OF THE INVENTION

### Problems to be Solved by the Invention

Patent Documents 1 and 2 describe metabolic pathways by which the microorganisms can produce 3-hydroxyadipic acid and α-hydromuconic acid, but have no description about interruption of the metabolic pathways to allow the microorganisms to secrete 3-hydroxyadipic acid or α-hydromuconic acid into culture medium. Moreover, the prior studies described in Patent Documents 1 to 4 have not examined whether or not 3-hydroxyadipic acid, α-hydromuconic acid, or adipic acid can be actually produced by using a non-natural microorganism in which a nucleic acid encoding an enzyme that catalyzes a reaction to reduce 3-oxoadipyl-CoA to 3-hydroxyadipyl-CoA has been introduced. Accordingly, it is not known whether the enzyme that catalyzes a reaction to reduce 3-oxoadipyl-CoA to 3-hydroxyadipyl-CoA, as described in Patent Documents 1 to 4, also exhibits excellent activity in the production of 3-hydroxyadipic acid, α-hydromuconic acid, andor adipic acid.

Accordingly, an object of the present invention is to provide a genetically modified microorganism for producing 3-hydroxyadipic acid, α-hydromuconic acid, andor adipic acid in high yield and a method of producing a substance by using the modified microorganism, wherein the modified microorganism is based on a genetically modified microorganism in which a nucleic acid encoding an enzyme that exhibits excellent activity in 3-oxoadipyl-CoA reduction reaction is introduced or the expression of the enzyme is enhanced, and wherein the modified microorganism is further modified to have an altered metabolic pathway.

### Means for Solving the Problem

The inventors intensively studied in order to achieve the above-described object and consequently found that 3-hydroxyadipic acid, a-hydromuconic acid, andor adipic acid can be produced in high yield by a genetically modified microorganism in which a nucleic acid encoding an enzyme that exhibits excellent activity in 3-oxoadipyl-CoA reduction reaction is introduced or the expression of the enzyme is enhanced and the function of pyruvate kinase is further impaired, to complete the present invention.

That is, the present invention provides the following:
(1) A genetically modified microorganism in which a nucleic acid encoding any one of the polypeptides described in (a) to (c) below is introduced or the expression of the polypeptide is enhanced and the function of pyruvate kinase is impaired:

(a) a polypeptide composed of an amino acid sequence represented by any one of SEQ ID NOs: 1 to 7;

(b) a polypeptide composed of the same amino acid sequence as that represented by any one of SEQ ID NOs: 1 to 7, except that one or several amino acids are substituted, deleted, inserted, andor added, and having an enzymatic activity that catalyzes a reaction to reduce 3-oxoadipyl-CoA to 3-hydroxyadipyl-CoA;

(c) a polypeptide composed of an amino acid sequence with a sequence identity of not less than 70% to the sequence represented by any one of SEQ ID NOs: 1 to 7 and having an enzymatic activity that catalyzes a reaction to reduce 3-oxoadipyl-CoA to 3-hydroxyadipyl-CoA.
(2) The genetically modified microorganism according to (1), wherein a polypeptide selected from the above (b) and (c) contains a region composed of an amino acid sequence represented by SEQ ID NO: 173.
(3) The genetically modified microorganism according to (2), wherein the amino acid sequence represented by SEQ ID NO: 173 contains a phenylalanine or leucine residue at the 13th amino acid position from the N terminus, a leucine or glutamine residue at the 15th amino acid position from the N terminus, a lysine or asparagine residue at the 16th amino acid position from the N terminus, a glycine or serine residue at the 17th amino acid position from the N terminus, a proline or arginine residue at the 19th amino acid position from the N terminus, and a leucine, methionine, or valine residue at the 21st amino acid position from the N terminus.
(4) The genetically modified microorganism according to any one of (1) to (3), which is a genetically modified microorganism belonging to a genus selected from the group consisting of Escherichia, Serratia, Hafnia, and Pseudomonas.
(5) The genetically modified microorganism according to any one of (1) to (4), which has an ability to generate 3-oxoadipyl-CoA and coenzyme A from acetyl-CoA and succinyl-CoA and an ability to generate 3-hydroxyadipic acid from 3-hydroxyadipyl-CoA.
(6) The genetically modified microorganism according to any one of (1) to (4), which has an ability to generate 3-oxoadipyl-CoA and coenzyme A from acetyl-CoA and succinyl-CoA, an ability to generate 2,3-dehydroadipyl-CoA from 3-hydroxyadipyl-CoA, and an ability to generate α-hydromuconic acid from 2,3-dehydroadipyl-CoA.
(7) The genetically modified microorganism according to any one of (1) to (4), which has an ability to generate 3-oxoadipyl-CoA and coenzyme A from acetyl-CoA and succinyl-CoA, an ability to generate 2,3-dehydroadipyl-CoA from 3-hydroxyadipyl-CoA, an ability to generate adipyl-CoA from 2,3-dehydroadipyl-CoA, and an ability to generate adipic acid from adipyl-CoA.
(8) The genetically modified microorganism according to any one of (1) to (7), wherein the function of a phosphotransferase system enzyme is further impaired.
(9) A method of producing 3-hydroxyadipic acid, comprising culturing the genetically modified microorganism according to any one of (1) to (5) and (8) in a culture medium containing a carbon source as a raw material for fermentation.
(10) A method of producing u-hydromuconic acid, comprising culturing the genetically modified microorganism according to any one of (1) to (4), (6) and (8) in a culture medium containing a carbon source as a raw material for fermentation.
(11) A method of producing adipic acid, comprising culturing the genetically modified microorganism according to any one of (1) to (4), (7) and (8) in a culture medium containing a carbon source as a raw material for fermentation.
(12) A method of producing one or more substances selected from the group consisting of 3-hydroxyadipic acid, α-hydromuconic acid, and adipic acid, comprising culturing a genetically modified microorganism in a culture medium containing a carbon source as a raw material for fermentation, wherein a nucleic acid encoding a polypeptide encoded by the 3-hydroxybutyryl-CoA dehydrogenase gene of a microorganism of the genus Serratia, which forms a gene cluster with 5-aminolevulinic acid synthase gene in the microorganism, is introduced or the expression of the polypeptide is enhanced and the function of pyruvate kinase is impaired in the genetically modified microorganism.
(13) The method according to (12), wherein the genetically modified microorganism is a microorganism in which the function of a phosphotransferase system enzyme is further impaired.

### Effects of the Invention

The genetically modified microorganism according to the present invention, which expresses an enzyme that exhibits excellent activity in a reaction to reduce 3-oxoadipyl-CoA to 3-hydroxyadipyl-CoA and, furthermore, has an impaired pyruvate kinase function, can produce 3-hydroxyadipic acid, α-hydromuconic acid, andor adipic acid in high yield compared to a parental strain of the microorganism in which pyruvate kinase is not impaired.

The method of producing a substance according to the present invention uses the genetically modified microorganism which is excellent in the production of 3-hydroxyadipic acid, α-hydromuconic acid, andor adipic acid via production of 3-hydroxyadipyl-CoA and thus can greatly increase the production of those substances.

### BRIEF DESCRIPTION OF THE DRAWING

FIG. 1 shows a gene cluster composed of a 3-hydroxybutyryl-CoA dehydrogenase gene and a 5-aminolevulinic acid synthase gene.

### DETAILED DESCRIPTION OF THE INVENTION

The microorganism according to the present invention is a genetically modified microorganism in which a nucleic acid encoding a polypeptide described in (a) to (c) below is introduced or the expression of the polypeptide is enhanced and the function of pyruvate kinase is impaired:

(a) a polypeptide composed of an amino acid sequence represented by any one of SEQ ID NOs: 1 to 7;

(b) a polypeptide composed of the same amino acid sequence as that represented by any one of SEQ ID NOs: 1 to 7, except that one or several amino acids are substituted, deleted, inserted, andor added, and having an enzymatic activity that catalyzes a reaction to reduce 3-oxoadipyl-CoA to 3-hydroxyadipyl-CoA;

(c) a polypeptide composed of an amino acid sequence with a sequence identity of not less than 70% to the sequence represented by any one of SEQ ID NOs: 1 to 7 and having activity in reduction of 3-oxoadipyl-CoA to 3-hydroxyadipyl-CoA.

An enzyme that catalyzes the reaction of reducing 3-oxoadipyl-CoA to 3-hydroxyadipyl-CoA is hereinafter referred to as “3-oxoadipyl-CoA reductase” in the specification. Additionally, 3-hydroxyadipic acid, α-hydromuconic acid, and adipic acid may be abbreviated as 3IIA, HMA, and ADA, respectively, in this specification.

In the present invention, introducing a nucleic acid means introducing a nucleic acid from the outside to the inside of a microorganism to give the microorganism an ability to produce a polypeptide encoded by the nucleic acid. The method of introduction of a nucleic acid is not limited to a particular method, and examples of the method that can be used include a method in which a nucleic acid of interest is integrated into an expression vector capable of autonomous replication in a microorganism and then integrated into a host microorganism, and a method in which a nucleic acid of interest is integrated into the genome of a microorganism.

In the present invention, enhancing the expression of a polypeptide means enhancing the expression of a polypeptide which a microorganism originally has. The method of enhancement of expression is not limited to a particular method, and examples of the method include a method in which a nucleic acid encoding a polypeptide of interest is increased in copy number, and a method in which a promoter region or a ribosome-binding sequence upstream of the region coding for a polypeptide of interest is modified. These methods may be carried out individually or in combination.

Additionally, one or more of the above nucleic acids may be introduced. Moreover, the introduction of a nucleic acid and the enhancement of polypeptide expression may be combined.

For the polypeptide used in the present invention and composed of the same amino acid sequence as that represented by any one of SEQ ID NOs: 1 to 7, except that one or several amino acids are substituted, deleted, inserted, andor added, and having 3-oxoadipyl-CoA reductase activity, the range represented by the phrase “one or several” is preferably 10 or less, more preferably 5 or less, especially preferably 4 or less, and most preferably 1 or 2 or less. In the case of amino acid substitution, the activity of the original polypeptide is more likely to be maintained when an amino acid(s) isare replaced by an amino acid(s) with similar properties (so-called conservative substitution). That is, the physiological properties of the original polypeptide are often maintained when an amino acid(s) isarc replaced by an amino acid(s) with similar properties. Therefore, in the case of substitution, a given amino acid is preferably replaced by another amino acid with similar properties. That is, the 20 amino acids that make up natural proteins can be divided into groups of amino acids with similar properties, such as neutral amino acids with a less polar side chain (Gly, Ile, Val, Leu. Ala, Met, Pro), neutral amino acids with a hydrophilic side chain (Asn, Gln, Thr, Ser, Tyr, Cys), acidic amino acids (Asp, Glu), and basic amino acids

(Arg, Lys, His), and aromatic amino acids (Phe, Tyr, Trp). It is often the case that substitution between amino acids in the same group does not change the properties of the original polypeptide.

For the polypeptide used in the present invention and having an amino acid sequence with a sequence identity of not less than 70% to the sequence represented by any one of SEQ ID NOs: 1 to 7 and having 3-oxoadipyl-CoA reductase activity, the sequence identity is preferably not less than 80%, more preferably not less than 85%, further preferably not less than 90%, still further preferably not less than 95%, yet further preferably not less than 97%, and even further preferably not less than 99%.

In the present invention, the term “sequence identity” means a ratio (percentage) of the number of identical amino acid or nucleotide residues relative to the total number of amino acid or nucleotide residues over the overlapping portion of an amino acid sequence alignment (including an amino acid corresponding to the translation start site) or a nucleotide sequence alignment (including the start codon), which is obtained by aligning two amino acid or nucleotide sequences with or without introduction of gaps for an optimal match, and is calculated by the following formula (1). In the formula (1), the length of a shorter sequence being compared is not less than 400 amino acids; in cases where the length of the shorter sequence is less than 400 amino acids, the sequence identity is not defined. The sequence identity can be easily determined using BLAST (Basic Local Alignment Search Tool), an algorithm widely used in this field. For example, BLAST is publicly available on a website, such as that of NCB1 (National Center for Biotechnology Information) or KEGG (Kyoto Encyclopedia of Genes and Genomes), on which the sequence identity can be easily determined using default parameters. Additionally, the sequence identity can also be determined using a similar function implemented in a software program such as Genetyx.

$$( 1 )$$

$$Sequence identity ( \% ) = the number of matches ( without counting the number of gaps ) \text{/} the length of a shorter sequence ( excluding the terminal gaps ) \times 100$$

By using a function of Genetyx (% Identity Matrix) to calculate sequence identities based on the formula (1) among the amino acid sequences represented by SEQ ID NOs: 1 to 7, the lowest sequence identity of 71.51% is found between the sequences represented by SEQ ID NOs: 2 and 4, and the sequence identities among the amino acid sequences represented by SEQ ID NOs: 1 to 7 are found to be at least not less than 70%. The results of calculation of sequence identity using Genetyx are presented in Table 1. In Tables 1 to 5 below, the numbers in the leftmost column represent SEQ ID NOs.

TABLE 1
[GENETYX: identity Matrix]
*Gaps are NOT taken into account.

| | 1 Serratia | 2 Serratia | 3 Serratia | 4 Serratia | 5 Serratia | 6 Serratia | 7 Serratia |
|---|---|---|---|---|---|---|---|
| [%] | | | | | | | |
| 1 Serratia marcescens ATCC13880 | * | | | | | | |
| 2 Serratia nematodiphila DSM21420 | 98.23 | * | | | | | |
| 3 Serratia plymuthica NBRC102599 | 72.10 | 71.51 | * | | | | |
| 4 Serratia proteamaculans 568 | 72.29 | 71.51 | 86.24 | * | | | |
| 5 Serratia ureilytica Lr5/4 | 90.76 | 90.76 | 72.88 | 73.28 | * | | |
| 6 Serratia sp. BW106 | 72.29 | 71.90 | 87.03 | 92.33 | 73.67 | * | |
| 7 Sierratia liquefaciens FK01 | 72.29 | 71.70 | 84.67 | 86.83 | 73.47 | 87.81 | * |
| [Match Count/Length] | | | | | | | |
| 1 Serratia marcescens ATCC13880 | * | | | | | | |
| 2 Serratia nematodiphila DSM21420 | 500/509 | * | | | | | |
| 3 Serratia plymuthica NBRC102599 | 367/509 | 364/509 | * | | | | |
| 4 Serratia proteamaculans 568 | 368/509 | 364/509 | 439/509 | * | | | |
| 5 Serratia ureilytica Lr5/4 | 462/509 | 462/509 | 371/509 | 373/509 | * | | |
| 6 Serratia sp. BW106 | 368/509 | 366/509 | 443/509 | 470/509 | 375/509 | * | |
| 7 Serratia liquefaciens FK01 | 368/509 | 365/509 | 431/509 | 442/509 | 374/509 | 447/509 | * |

When each of the amino acid sequences represented by SEQ ID NOs: 1 to 7 as queries was compared using BLASTP to all the amino acid sequences registered in the NCBI amino acid database (non-redundant protein sequences) to determine sequence identities, all sequences with a sequence identity of not less than 70% were found to be from bacteria of the genus Serrano.

All the polypeptides represented by SEQ ID NOs: 1 to 7 as described above in (a) contain a common sequence 1 composed of 24 amino acid residues and represented by SEQ ID NO: 173 within a region from the 1 5th to the 38th amino acid residues from the N terminus (hereinafter, an amino acid residue at the n-th position from the N terminus may conveniently be represented by n “a.a.”; for example, the region from the 15th to the 38th amino acid residues from the N tei tinus may be thus simply represented by “15 to 38 a.a.”). In the common sequence 1, Xaa represents an arbitrary amino acid residue, and the 13 a.a. is preferably a phenylalanine or leucine, and the 15 a.a. is preferably a leucine or glutamine, and the 16 a.a. is preferably a lysine or asparagine, and the 17 a.a. is a glycine or serine, more preferably a glycine, and the 19 a.a. is preferably a proline or arginine, and the 21 a.a. is preferably a leucine, methionine, or valine. The common sequence 1 corresponds to the region including the NADtbinding residue and the surrounding amino acid residues. In the NAD^{±}-binding residues, the 24th amino acid residue in the common sequence 1 is an aspartic acid, as described in Biochimie., 2012 Feb. 94 (2): 471-8., but in the common sequence 1, the residue is an asparagine, which is characteristic. It is thought that the presence of the common sequence 1 causes the polypeptides represented by SEQ ID NOs: 1 to 7 to show excellent enzymatic activity as 3-oxoadipyl-CoA reductases.

The polypeptides as described above in (b) and (c) also preferably contain the common sequence 1 composed of 24 amino acid residues and represented by SEQ ID NO: 173 within a region from 1 to 200 a.a. The common sequence is more preferably located within a region from 1 to 150 a.a., and further preferably within a region from 1 to 100 a.a. Specific examples of the polypeptides include those with the amino acid sequences represented by SEQ ID NOs: 8 to 86. The amino acid sequences represented by SEQ ID NOs: 8 to 86 contain the common sequence 1 composed of 24 amino acid residues and represented by SEQ ID NO: 173 within a region from 15 to 38 a.a. The amino acid sequences represented by SEQ ID NOs: 8 to 86 have a sequence identity of not less than 90% to the amino acid sequence represented by any one of SEQ ID NOs: 1 to 7. The results of calculation of sequence identity using Genetyx are presented in Tables 2-1 to 2-3 and Tables 3-1 to 3-3.

TABLE 2-1
[GENETYX: 1 Identity Matrix]
*Gaps are NOT taken into account.

| [%] | | 1 Serratia | 2 Serratia | 3 Serratia | 4 Serratia | 5 Serratia | 6 Serratia | 7 Serratia |
|---|---|---|---|---|---|---|---|---|
| 1 | Serratia marcescens ATCC13880 | * | | | | | | |
| 2 | Serratia nematodiphila DSM21420 | 98.23 | * | | | | | |
| 3 | Serratia plymuthica NBRC102599 | 72.10 | 71.51 | * | | | | |
| 4 | Serratia proteamaculans 568 | 72.29 | 71.51 | 86.24 | * | | | |
| 5 | Serratia ureilytica Lr5/4 | 90.76 | 90.76 | 72.88 | 73.28 | * | | |
| 6 | Serratia sp. BW106 | 72.29 | 71.90 | 87.03 | 92.33 | 73.67 | * | |
| 7 | Serratia liquefaciens FK01 | 72.29 | 71.70 | 84.67 | 86.83 | 73.47 | 87.81 | * |
| 8 | Serratia sp. S119 | 94.89 | 94.30 | 72.88 | 72.49 | 91.55 | 73.08 | 72.88 |
| 9 | Serratia sp. YD25 | 92.33 | 92.33 | 72.49 | 72.49 | 93.51 | 72.69 | 72.88 |
| 10 | Serratia sp. FS14 | 98.62 | 99.60 | 71.70 | 71.70 | 91.15 | 72.10 | 72.10 |
| 11 | Serratia sp. HMSC15F11 | 94.89 | 94.30 | 73.28 | 73.28 | 91.35 | 73.47 | 73.47 |
| 12 | Serratia sp. JKS000199 | 90.76 | 90.76 | 72.69 | 73.08 | 99.41 | 73.47 | 73.28 |
| 13 | Serratia sp. TEL | 90.56 | 90.56 | 72.88 | 73.28 | 99.80 | 73.67 | 73.47 |
| 14 | Serratia sp. ISTD04 | 90.56 | 90.56 | 72.49 | 73.08 | 99.41 | 73.47 | 73.28 |
| 15 | Serratia sp. SCB1 | 90.76 | 90.76 | 72.88 | 73.28 | 99.60 | 73.47 | 73.47 |
| 16 | Serratia sp. S4 | 72.10 | 71.31 | 86.44 | 98.62 | 73.08 | 91.94 | 86.64 |
| 17 | Serratia sp. C-1 | 72.49 | 71.90 | 98.03 | 86.05 | 73.28 | 86.64 | 84.08 |
| 18 | Serratia marcescens 532 | 99.80 | 98.03 | 72.29 | 72.10 | 90.56 | 72.10 | 72.10 |
| 19 | Serratia marcescens 2880STDY5683033 | 99.60 | 97.83 | 72.10 | 72.29 | 90.37 | 72.10 | 72.29 |
| 20 | Serratia marcescens WW4 | 98.42 | 99.41 | 71.90 | 71.90 | 90.96 | 72.29 | 71.90 |
| 21 | Serratia marcescens K27 | 98.23 | 99.21 | 71.31 | 71.31 | 90.96 | 71.70 | 71.70 |
| 22 | Serratia marcescens 280 | 98.42 | 99.41 | 71.70 | 71.70 | 90.96 | 72.10 | 72.10 |
| 23 | Serratia marcescens 19F | 98.42 | 99.41 | 71.51 | 71.70 | 90.96 | 72.10 | 72.10 |
| 24 | Serratia marcescens 1185 | 98.23 | 99.60 | 71.31 | 71.31 | 90.37 | 71.70 | 71.51 |

TABLE 2-2

| | | | | | | | | |
|---|---|---|---|---|---|---|---|---|
| 25 | Serratia marcescens S217 | 98.23 | 99.21 | 71.31 | 71.51 | 90.96 | 71.90 | 71.90 |
| 26 | Serratia marcescens KHCo-24B | 98.03 | 99.80 | 71.31 | 71.31 | 90.56 | 71.70 | 71.90 |
| 27 | Serratia marcescens Z6 | 98.03 | 99.01 | 71.70 | 71.90 | 90.56 | 72.29 | 71.90 |
| 28 | Serratia marcescens 546 | 97.83 | 99.21 | 71.51 | 71.70 | 90.37 | 72.10 | 71.70 |
| 29 | Serratia nematodiphila HB307 | 98.03 | 99.80 | 71.31 | 71.51 | 90.56 | 71.90 | 71.70 |
| 30 | Serratia marcescens VGH107 | 98.03 | 99.01 | 71.31 | 71.51 | 90.56 | 71.90 | 71.90 |
| 31 | Serratia marcescens MCB | 95.48 | 95.28 | 72.29 | 72.69 | 91.15 | 72.88 | 72.69 |
| 32 | Serratia marcescens AH0650 | 95.67 | 95.48 | 72.29 | 72.69 | 90.76 | 73.28 | 72.69 |
| 33 | Serratia marcescens UMH12 | 95.48 | 95.28 | 72.10 | 72.49 | 90.56 | 73.08 | 72.49 |
| 34 | Serratia sp. OMLW3 | 95.48 | 95.28 | 72.29 | 72.49 | 90.76 | 73.28 | 72.69 |
| 35 | Serratia marcescens UMH11 | 95.28 | 95.08 | 72.10 | 72.69 | 90.56 | 73.47 | 72.49 |
| 36 | Serratia marcescens UMH1 | 95.08 | 94.89 | 72.29 | 72.49 | 90.17 | 73.08 | 72.29 |
| 37 | Serratia marcescens 2880STDY5683020 | 95.48 | 94.89 | 73.08 | 72.69 | 92.14 | 73.28 | 73.08 |
| 38 | Serratia marcescens 99 | 95.48 | 94.69 | 73.28 | 72.88 | 91.55 | 73.67 | 73.28 |
| 39 | Serratia marcescens 374 | 94.89 | 94.69 | 72.29 | 72.29 | 90.17 | 73.08 | 72.29 |
| 40 | Serratia marcescens 2880STDY5683036 | 95.28 | 94.49 | 73.08 | 72.69 | 91.35 | 73.47 | 73.08 |
| 41 | Serratia marcescens 2880STDY5683034 | 95.28 | 94.69 | 73.08 | 72.69 | 91.94 | 73.28 | 73.08 |
| 42 | Serratia marcescens 2880STDY5682892 | 95.28 | 94.69 | 73.28 | 72.88 | 91.94 | 73.47 | 73.28 |
| 43 | Serratia marcescens SM39 | 95.08 | 94.49 | 73.28 | 72.69 | 92.14 | 73.28 | 73.28 |
| 44 | Serratia marcescens 189 | 95.08 | 94.49 | 73.28 | 72.88 | 92.14 | 73.47 | 73.28 |
| 45 | Serratia marcescens SMB2099 | 95.08 | 94.49 | 73.47 | 72.69 | 91.74 | 73.67 | 73.47 |
| 46 | Serratia marcescens 2880STDY5682862 | 94.89 | 94.30 | 73.47 | 72.88 | 91.55 | 73.47 | 73.47 |
| 47 | Serratia marcescens SE4145 | 94.89 | 94.30 | 73.08 | 72.49 | 91.94 | 73.08 | 73.08 |
| 48 | Serratia marcescens 2880STDY5682876 | 95.08 | 94.49 | 73.28 | 72.88 | 91.74 | 73.47 | 73.28 |
| 49 | Serratia marcescens 709 | 95.08 | 94.49 | 73.08 | 72.69 | 91.74 | 73.28 | 73.08 |
| 50 | Serratia marcescens MGH136 | 94.89 | 94.30 | 72.88 | 72.49 | 91.94 | 73.08 | 72.88 |
| 51 | Serratia marcescens 2880STDY5682884 | 94.69 | 94.10 | 72.88 | 72.49 | 91.74 | 73.08 | 73.08 |
| 52 | Serratia marcescens D-3 | 95.08 | 94.49 | 73.08 | 72.69 | 91.74 | 73.28 | 73.08 |
| 53 | Serratia marcescens 2880STDY5682957 | 94.89 | 94.30 | 72.88 | 72.69 | 91.55 | 73.28 | 72.88 |
| 54 | Serratia marcescens YDC563 | 94.69 | 94.10 | 72.88 | 72.69 | 91.35 | 73.28 | 72.88 |
| 55 | Serratia marcescens 2880STDY5683035 | 94.80 | 94.30 | 73.08 | 72.69 | 91.55 | 73.28 | 73.08 |

TABLE 2-3

| | | | | | | | | |
|---|---|---|---|---|---|---|---|---|
| 56 | Serratia marcescens 2880STDY5682930 | 94.69 | 94.10 | 72.88 | 72.49 | 91.35 | 73.08 | 72.88 |
| 57 | Serratia marcescens 790 | 94.49 | 94.30 | 73.28 | 72.88 | 91.35 | 73.47 | 73.28 |
| 58 | Serratia marcescens UMH5 | 93.51 | 92.92 | 72.69 | 72.88 | 90.37 | 72.69 | 72.49 |
| 59 | Serratia marcescens 288OSTDY5682988 | 93.32 | 92.73 | 72.69 | 72.88 | 90.17 | 72.69 | 72.49 |
| 60 | Serratia marcescens 945154301 | 94.89 | 94.30 | 73.28 | 73.28 | 91.35 | 73.67 | 73.47 |
| 61 | Serratia marcescens at10508 | 94.69 | 94.10 | 73.47 | 73.47 | 91.15 | 73.67 | 73.67 |
| 62 | Serratia marcescens ML2637 | 94.49 | 93.90 | 73.28 | 73.47 | 90.96 | 73.67 | 73.67 |
| 63 | Serratia marcescens SM1978 | 94.30 | 93.71 | 73.28 | 73.28 | 90.76 | 73.67 | 73.67 |
| 64 | Serratia marcescens PWN146 | 94.10 | 93.51 | 72.88 | 72.88 | 90.96 | 72.88 | 73.28 |
| 65 | Serratia marcescens H1q | 92.53 | 92.53 | 72.49 | 72.49 | 93.51 | 72.69 | 73.08 |
| 66 | Serratia marcescens UMH6 | 91.15 | 91.15 | 72.69 | 73.08 | 99.60 | 73.47 | 73.28 |
| 67 | Serratia nematodiphila WCU338 | 91.15 | 91.15 | 72.69 | 73.08 | 99.41 | 73.47 | 73.28 |
| 68 | Serratia sp. OLEL1 | 90.96 | 90.96 | 72.88 | 73.28 | 99.80 | 73.67 | 73.47 |
| 69 | Serratia marcescens 7209 | 90.96 | 90.96 | 72.49 | 72.88 | 99.41 | 73.28 | 73.08 |
| 70 | Serratia marcescens sicaria (Ss1) | 90.96 | 90.96 | 72.69 | 73.08 | 99.41 | 73.28 | 73.28 |
| 71 | Serratia sp. OLFL2 | 90.76 | 90.76 | 72.69 | 73.08 | 99.60 | 73.47 | 73.28 |
| 72 | Serratia marcescens BIDMC 81 | 90.76 | 90.76 | 72.88 | 73.28 | 99.60 | 73.67 | 73.47 |
| 73 | Serratia marcescens BIDMC 50 | 90.76 | 90.76 | 72.69 | 73.08 | 99.21 | 73.47 | 73.28 |
| 74 | Serratia marcescens UMH7 | 90.56 | 90.56 | 72.88 | 73.28 | 99.80 | 73.67 | 73.47 |
| 75 | Serratia marcescens RSC-14 | 90.56 | 90.56 | 72.88 | 73.47 | 99.21 | 73.87 | 73.67 |
| 76 | Serratia marcescens SIMO3 | 92.33 | 92.33 | 72.29 | 72.29 | 93.51 | 72.49 | 72.88 |
| 77 | Serratia marcescens 90-166 | 90.17 | 89.78 | 72.49 | 73.47 | 96.66 | 73.67 | 73.08 |
| 78 | Serratia marcescens UMH2 | 90.76 | 90.76 | 72.88 | 73.28 | 99.21 | 73.67 | 73.47 |
| 79 | Serratia plymuthica A30 | 72.49 | 71.90 | 96.66 | 85.06 | 73.47 | 86.05 | 83.69 |
| 80 | Serratia plymuthica tumart 205 | 72.69 | 72.10 | 98.03 | 86.24 | 73.47 | 86.64 | 84.28 |
| 81 | Serratia plymuthica A30 | 72.29 | 71.70 | 98.82 | 85.65 | 72.88 | 86.44 | 84.08 |
| 82 | Serratia plymuthica 4Rx13 | 72.29 | 71.70 | 97.83 | 85.85 | 73.08 | 86.44 | 84.28 |
| 83 | Serratia plymuthica V4 | 72.29 | 71.70 | 98.42 | 85.85 | 71.08 | 86.44 | 84.28 |
| 84 | Serratia plymuthica 3Rp8 | 72.29 | 71.70 | 98.62 | 86.05 | 73.08 | 86.64 | 84.08 |
| 85 | Serratia proteamaculans MFPA44A14 | 72.29 | 71.90 | 87.03 | 92.53 | 73.28 | 98.82 | 87.22 |
| 86 | Serratia plymuthica A153 | 72.10 | 71.51 | 99.21 | 86.05 | 72.88 | 86.64 | 84.47 |

TABLE 3-1

| [Match Count/Length] | | 1 Serratia | 2 Serratia | 3 Serratia | 4 Serratia | 5 Serratia | 6 Serratia | 7 Serratia |
|---|---|---|---|---|---|---|---|---|
| 1 | Serratia marcescens ATCC13880 | * | | | | | | |
| 2 | Serratia nematodiphila DSM21420 | 500/509 | * | | | | | |
| 3 | Serratia plymuthica NBRC102599 | 367/509 | 364/509 | * | | | | |
| 4 | Serratia proteamaculans 568 | 368/509 | 364/509 | 439/509 | * | | | |
| 5 | Serratia ureilytica Lr5/4 | 482/509 | 462/509 | 371/500 | 373/509 | * | | |
| 6 | Serratia sp. BW106 | 368/509 | 366/509 | 443/509 | 470/509 | 375/509 | * | |
| 7 | Serratia liquefaciens FK01 | 368/509 | 365/509 | 431/509 | 442/509 | 374/509 | 447/509 | * |
| 8 | Serratia sp. S119 | 483/509 | 480/509 | 371/509 | 369/509 | 466/509 | 372/509 | 371/509 |
| 9 | Serratia sp. YD25 | 470/509 | 470/509 | 369/509 | 369/509 | 476/509 | 370/509 | 371/509 |
| 10 | Serratia sp. FS14 | 502/509 | 507/509 | 365/509 | 365/509 | 464/509 | 367/509 | 367/509 |
| 11 | Serratia sp. HMSC15F11 | 483/509 | 480/509 | 373/509 | 373/509 | 465/509 | 374/509 | 374/509 |
| 12 | Serratia sp. JKS000199 | 402/509 | 462/509 | 370/509 | 372/509 | 506/509 | 374/509 | 373/509 |
| 13 | Serratia sp. TEL | 461/509 | 461/509 | 371/509 | 373/509 | 508/508 | 375/509 | 374/509 |
| 14 | Serratia sp. ISTD04 | 461/509 | 461/509 | 369/509 | 372/509 | 506/509 | 374/509 | 373/509 |
| 15 | Serratia sp. SCBI | 462/509 | 462/509 | 371/509 | 373/509 | 507/509 | 374/509 | 374/509 |
| 16 | Serratia sp. S4 | 367/509 | 363/509 | 440/509 | 502/509 | 372/509 | 468/509 | 441/509 |
| 17 | Serratia sp. C-1 | 369/509 | 366/509 | 499/509 | 438/509 | 373/509 | 441/509 | 428/509 |
| 18 | Serratia marcescens 532 | 508/509 | 499/509 | 368/509 | 367/509 | 461/509 | 367/509 | 367/509 |
| 19 | Serratia marcescens 2880STDY5683033 | 507/509 | 498/509 | 367/509 | 368/509 | 460/509 | 367/509 | 368/509 |
| 20 | Serratia marcescens WW4 | 501/509 | 506/509 | 366/509 | 366/509 | 463/509 | 368/509 | 366/509 |
| 21 | Serratia marcescens K27 | 500/509 | 505/509 | 363/509 | 363/509 | 463/509 | 365/509 | 365/509 |
| 22 | Serratia marcescens 280 | 501/509 | 506/509 | 365/509 | 365/509 | 463/509 | 367/509 | 367/509 |
| 23 | Serratia marcescens 19F | 501/509 | 506/509 | 364/509 | 365/509 | 463/509 | 367/509 | 367/509 |
| 24 | Serratia marcescens 1185 | 500/509 | 507/509 | 363/509 | 363/509 | 460/509 | 365/509 | 364/509 |

TABLE 3-2

| | | | | | | | | |
|---|---|---|---|---|---|---|---|---|
| 25 | Serratia marcescens S217 | 500/509 | 505/509 | 363/509 | 364/509 | 463/509 | 366/509 | 366/509 |
| 26 | Serratia marcescens KHCo-24B | 499/509 | 508/509 | 363/509 | 363/509 | 461/509 | 365/509 | 366/509 |
| 27 | Serratia marcescens Z6 | 499/509 | 504/509 | 365/509 | 366/509 | 461/509 | 368/509 | 366/509 |
| 28 | Serratia marcescens 546 | 498/509 | 505/509 | 364/509 | 365/509 | 460/509 | 367/509 | 365/509 |
| 29 | Serratia nematodiphila MB307 | 499/509 | 508/509 | 363/509 | 364/509 | 461/509 | 366/509 | 365/509 |
| 30 | Serratia marcescens VGH107 | 499/509 | 504/509 | 363/509 | 364/509 | 461/509 | 366/509 | 366/509 |
| 31 | Serratia marcescens MCB | 486/509 | 485/509 | 368/509 | 370/509 | 464/509 | 371/509 | 370/509 |
| 32 | Serratia marcescens AH0650 | 487/509 | 486/509 | 368/509 | 370/509 | 462/509 | 373/509 | 370/509 |
| 33 | Serratia marcescens UMH12 | 486/509 | 485/509 | 367/509 | 369/509 | 461/509 | 372/509 | 369/509 |
| 34 | Serratia sp. OMLW3 | 486/509 | 485/509 | 368/509 | 369/509 | 462/509 | 373/509 | 370/509 |
| 35 | Serratia marcescens UMH11 | 485/509 | 484/509 | 367/509 | 370/509 | 461/509 | 374/509 | 369/509 |
| 36 | Serratia marcescens UMH1 | 484/509 | 483/509 | 368/509 | 369/509 | 459/509 | 372/509 | 368/509 |
| 37 | Serratia marcescens 2880STDY568320 | 486/509 | 483/509 | 372/509 | 370/509 | 469/509 | 373/509 | 372/509 |
| 38 | Serratia marcescens 99 | 486/509 | 482/509 | 373/509 | 371/509 | 466/509 | 375/509 | 373/509 |
| 39 | Serratia marcescens 374 | 483/509 | 482/509 | 368/509 | 368/509 | 459/509 | 372/509 | 368/509 |
| 40 | Serratia marcescens 2880STDY5683036 | 485/509 | 481/509 | 372/509 | 370/509 | 465/509 | 374/509 | 372/509 |
| 41 | Serratia marcescens 2880STDY5683034 | 485/509 | 482/509 | 372/509 | 370/509 | 468/509 | 373/509 | 372/509 |
| 42 | Serratia marcescens 2880STDY5682892 | 485/509 | 482/509 | 373/509 | 371/509 | 468/509 | 374/509 | 373/509 |
| 43 | Serratia marcescens SM39 | 484/509 | 481/509 | 373/509 | 370/509 | 469/509 | 373/509 | 373/509 |
| 44 | Serratia marcescens 189 | 484/509 | 481/509 | 373/509 | 371/509 | 469/509 | 374/509 | 373/509 |
| 45 | Serratia marcescens SMB2099 | 484/509 | 481/509 | 374/509 | 370/509 | 467/509 | 375/509 | 374/509 |
| 46 | Serratia marcescens 2880STDY5682862 | 483/509 | 480/509 | 374/509 | 371/509 | 466/509 | 374/509 | 374/509 |
| 47 | Serratia marcescens SE4145 | 483/509 | 480/509 | 372/509 | 369/509 | 468/509 | 372/509 | 372/509 |
| 48 | Serratia marcescens 2880STDY5682876 | 484/509 | 481/509 | 373/509 | 371/509 | 467/509 | 374/509 | 373/500 |
| 49 | Serratia marcescens 709 | 484/509 | 481/509 | 372/509 | 370/509 | 467/509 | 373/509 | 372/509 |
| 50 | Serratia marcescens MGH136 | 483/509 | 480/509 | 371/509 | 369/509 | 468/509 | 372/509 | 371/509 |
| 51 | Serratia marcescens 2880STDY5682884 | 482/509 | 479/509 | 371/509 | 369/509 | 467/509 | 372/509 | 372/509 |
| 52 | Serratia marcescens D-3 | 484/509 | 481/509 | 372/509 | 370/509 | 467/509 | 373/509 | 372/509 |
| 53 | Serratia marcescens 2880STDY5682957 | 483/509 | 480/509 | 371/509 | 370/509 | 466/509 | 373/509 | 371/509 |
| 54 | Serratia marcescens YDC563 | 482/509 | 479/509 | 371/509 | 370/509 | 465/509 | 373/509 | 371/509 |
| 55 | Serratia marcescens 2880STDY5683035 | 483/509 | 480/509 | 372/509 | 370/509 | 466/509 | 373/509 | 372/509 |

TABLE 3-3

| | | | | | | | | |
|---|---|---|---|---|---|---|---|---|
| 56 | Serratia marcescens 2880STDY5682930 | 482/509 | 479/509 | 371/509 | 369/509 | 465/509 | 372/509 | 371/509 |
| 57 | Serratia marcescens 790 | 481/509 | 480/509 | 373/509 | 371/509 | 465/509 | 374/509 | 373/509 |
| 58 | Serratia marcescens UMH5 | 476/509 | 473/509 | 370/509 | 371/509 | 460/509 | 370/509 | 369/509 |
| 59 | Serratia marcescens 2880STDY5682988 | 475/509 | 472/509 | 370/509 | 371/509 | 459/509 | 370/509 | 389/509 |
| 60 | Serratia marcescens 945154301 | 483/509 | 480/509 | 373/509 | 373/509 | 465/509 | 375/509 | 374/509 |
| 61 | Serratia marcescens at 10508 | 482/509 | 479/509 | 374/509 | 374/509 | 464/509 | 375/509 | 375/509 |
| 62 | Serratia marcescens ML2637 | 481/509 | 478/509 | 373/509 | 374/509 | 463/509 | 375/509 | 375/509 |
| 63 | Serratia marcescens SM1978 | 480/509 | 477/509 | 373/509 | 373/509 | 462/509 | 375/509 | 375/509 |
| 64 | Serratia marcescens PWN146 | 479/509 | 476/509 | 371/509 | 371/509 | 463/509 | 371/509 | 373/509 |
| 65 | Serratia marcescens H1q | 471/509 | 471/509 | 369/509 | 369/509 | 476/509 | 370/509 | 372/509 |
| 66 | Serratia marcescens UMH6 | 464/509 | 464/509 | 370/509 | 372/539 | 507/509 | 374/509 | 373/509 |
| 67 | Serratia nematodiphila WCU338 | 464/509 | 464/509 | 370/509 | 372/509 | 506/509 | 374/509 | 373/509 |
| 68 | Serratia sp. OLEL1 | 463/509 | 463/509 | 371/509 | 373/509 | 508/509 | 375/509 | 374/509 |
| 69 | Serratia marcescens 7209 | 463/509 | 463/509 | 369/509 | 371/509 | 506/509 | 373/509 | 372/509 |
| 70 | Serratia marcescens sicaria (Ss1) | 463/509 | 463/509 | 370/509 | 372/509 | 506/509 | 373/509 | 373/509 |
| 71 | Serratia sp. OLFL2 | 462/509 | 462/509 | 370/509 | 372/509 | 507/509 | 374/509 | 373/509 |
| 72 | Serratia marcescens BIDMC 81 | 462/509 | 462/509 | 371/509 | 373/509 | 507/509 | 375/509 | 374/509 |
| 73 | Serratia marcescens BIDMC 50 | 462/509 | 462/509 | 370/509 | 372/509 | 505/509 | 374/509 | 373/509 |
| 74 | Serratia marcescens UMH7 | 461/509 | 461/509 | 371/509 | 373/509 | 508/509 | 375/509 | 374/509 |
| 75 | Serratia marcescens RSC-14 | 461/509 | 461/509 | 371/509 | 374/509 | 505/509 | 376/509 | 375/509 |
| 76 | Serratia marcescens SMO3 | 470/509 | 470/509 | 368/509 | 368/509 | 476/509 | 369/509 | 371/509 |
| 77 | Serratia marcescens 90-166 | 459/509 | 457/509 | 369/509 | 374/509 | 492/509 | 375/509 | 372/509 |
| 78 | Serratia marcescens UMH2 | 462/509 | 462/509 | 371/509 | 373/509 | 505/509 | 375/509 | 374/509 |
| 79 | Serratia plymuthica AS9 | 369/509 | 366/509 | 492/509 | 433/509 | 374/509 | 438/509 | 426/509 |
| 80 | Serratia plymuthica tumat 205 | 370/509 | 367/509 | 499/509 | 439/509 | 374/509 | 441/509 | 429/509 |
| 81 | Serratia plymuthica A30 | 368/509 | 365/509 | 503/509 | 436/509 | 371/509 | 440/509 | 428/509 |
| 82 | Serratia plymuthica 4Rx13 | 368/509 | 365/509 | 498/509 | 437/509 | 372/509 | 440/509 | 429/509 |
| 83 | Serratia plymuthica V4 | 368/509 | 365/509 | 501/509 | 437/509 | 372/509 | 440/509 | 429/509 |
| 84 | Serratia plymuthica 3Rp8 | 368/509 | 365/509 | 502/509 | 438/509 | 372/509 | 441/509 | 428/509 |
| 85 | Serratia proteamaculans MFPA44A14 | 368/509 | 366/509 | 443/509 | 471/509 | 373/509 | 503/509 | 444/509 |
| 86 | Serratia plymuthica A153 | 367/509 | 364/509 | 505/509 | 438/509 | 371/509 | 441/509 | 430/509 |

The nucleic acids encoding the polypeptides described in (a) to (c) according to the present invention may contain an additional sequence that encodes a peptide or protein added to the original polypeptides at the N terminus andor the C terminus. Examples of such a peptide or protein can include secretory signal sequences, translocation proteins, binding proteins, peptide tags for purification, and fluorescent proteins. Among those peptides or proteins, a peptide or protein with a desired function can be selected depending on the purpose and can be added to the polypeptides of the present invention by those skilled in the art. It should be noted that the amino acid sequence of such a peptide or protein is excluded from the calculation of sequence identity.

The nucleic acids encoding the polypeptides represented by SEQ ID NOs: 1 to 86 are not specifically limited, provided that the nucleic acids have nucleotide sequences that can be translated to the amino acid sequences represented by SEQ ID NOs: 1 to 86, and the nucleotide sequences can be deteunined considering the set of codons (standard genetic code) corresponding to each amino acid. In this respect, the nucleotide sequences may be redesigned using codons that are frequently used by a host microorganism used in the present invention.

Specific examples of the nucleotide sequences of the nucleic acids that encode the polypeptides with the amino acid sequences represented by SEQ ID NOs: 1 to 86 include the nucleotide sequences represented by SEQ ID NOs: 87 to 172.

In the present invention, whether or not a polypeptide encoded by a certain nucleic acid has 3-oxoadipyl-CoA reductase activity is determined as follows: transformants A and B below are produced and grown in a culture test; if 3-hydroxyadipic acid or α-hydromuconic acid is confirmed in the resulting culture medium, it is judged that the nucleic acid encodes a polypeptide having 3-oxoadipyl-CoA reductase activity. The determination method will be described using the scheme 1 below which shows a biosynthesis pathway.

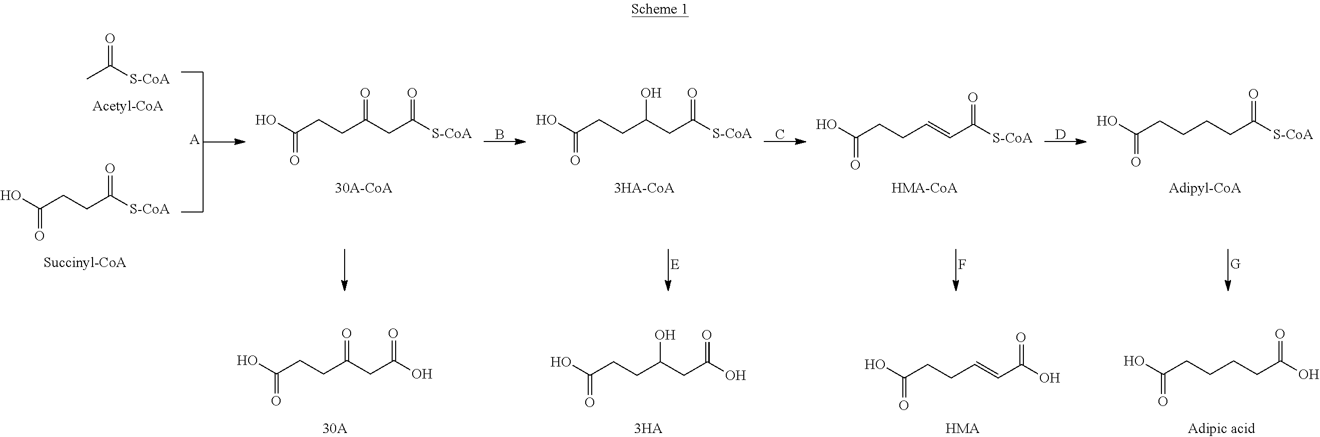

The above scheme 1 shows an exemplary reaction pathway required for the production of 3-hydroxyadipic acid, α-hydromuconic acid, andor adipic acid. In this scheme, the reaction A represents a reaction that generates 3-oxoadipyl-CoA and coenzyme A from acetyl-CoA and succinyl-CoA. The reaction B represents a reaction that generates 3-hydroxyadipyl-CoA from 3-oxoadipyl-CoA. The reaction C represents a reaction that generates 2,3-dehydroadipyl-CoA from 3-hydroxyadipyl-CoA. The reaction D represents a reaction that generates adipyl-CoA from 2,3-dehydroadipyl-CoA. The reaction E represents a reaction that generates 3-hydroxyadipic acid from 3-hydroxyadipyl-CoA. The reaction F represents a reaction that generates α-hydromuconic acid from 2,3-dehydroadipyl-CoA. The reaction G represents a reaction that generates adipic acid from adipyl-CoA.

The transformant A has enzymes that catalyze the reactions A, E, and F. The transformant B has enzymes that catalyze the reactions A, C, E, and F.

The transformant A is first produced. Plasmids fbr the expression of the enzymes that catalyze the reactions A, E, and F, respectively, are produced. The reactions E and F can be catalyzed by an identical enzyme. The plasmids are introduced into Escherichia coli strain BL21 (DE3), which is a microorganism strain lacking abilities to produce all of 3-hydroxyadipic acid, α-hydromuconic acid, and adipic acid. Into the obtained transformant, an expression plasmid carrying a nucleic acid that encodes a polypeptide to be analyzed for the presence of the enzymatic activity of interest and is integrated downstream of an appropriate promoter is introduced to obtain the transformant A. The transformant A is cultured, and the post-culture fluid is examined for the presence of 3-hydroxyadipic acid. Once the presence of 3-hydroxyadipic acid in the culture fluid is confirmed, the transformant B is then produced. The transformant B is obtained by producing a plasmid for the expression of an enzyme that catalyzes the reaction C and introducing the resulting plasmid into the transformant A. The transformant B is cultured, and the post-culture fluid is examined for the presence of α-hydromuconic acid. When the presence of α-hydromuconic acid in the post-culture fluid is confirmed, it indicates that 3-hydroxyadipic acid produced in the transformant A and α-hydromuconic acid produced in the transformant B are generated via production of 3-hydroxyadipyl-CoA, and that the polypeptide of interest has 3-oxoadipyl-CoA reductase activity.

As the gene encoding the enzyme that catalyzes the reaction A, pcaF from Pseudomonas putida strain KT2440 (NCBI Gene ID: 1041755; SEQ ID NO: 174) is used.

As the genes encoding the enzyme that catalyzes the reactions E and F, a continuous sequence including the full lengths of peal and peal from Pseudomonas putida strain KT2440 (NCBI Gene IDs: 1046613 and 1046612; SEQ ID NOs: 175 and 176) is used. The polypeptides encoded by peal and peal forms a complex and then catalyze the reactions E and F.

As the nucleic acid encoding the enzyme that catalyzes the reaction C, the paaF gene from Pseudomonas putida strain KT2440 (NCBI Gene ID: 1046932, SEQ ID NO: 177) is used.

The method of culturing the transformant A and the transformant B is as follows. Antibiotics for stable maintenance of the plasmids and inducer substances for induction of expression of the polypeptides encoded by the incorporated nucleic acids may be added as appropriate to the culture. A loopful of either the transformant A or B is inoculated into 5 mL of the culture medium I (10 gL Bacto Tryptone (manufactured by Difco Laboratories), 5 gL Bacto Yeast Extract (manufactured by Difco Laboratories), 5 gL sodium chloride) adjusted at pH 7 and is cultured at 30° C. with shaking at 120 min⁻¹ for 18 hours to prepare a preculture fluid. Subsequently, 0.25 mL of the preculture fluid is added to 5 mL of the culture medium II (10 gL succinic acid, 10 gL glucose, 1 gL ammonium sulfate, 50 mM potassium phosphate, 0.025 gL magnesium sulfate, 0.0625 mgL iron sulfate, 2.7 mgL manganese sulfate, 0.33 mgL calcium chloride, 1.25 gL sodium chloride, 2.5 gL Bacto Tryptone, 1.25 gL Bacto Yeast Extract) adjusted to pH 6.5 and is cultured at 30° C. with shaking at 120 min⁻¹ for 24 hours. The obtained culture fluid is examined for the presence of 3-hydroxyadipic acid or α-hydromuconic acid.

The presence of 3-hydroxyadipic acid or α-hydromuconic acid in the culture fluid can be confirmed by centrifuging the culture fluid and analyzing the supernatant with LC-MSMS. The analysis conditions are as described below:
HPLC: 1290 Infinity (manufactured by Agilent Technologies, Inc.)
Column: Synergi hydro-RP (manufactured by Phenomenex Inc.), length: 100 mm,
internal diameter: 3 mm, particle size: 2.5 μm
Mobile phase: 0.1% aqueous formic acid solution methanol =7030
Flow rate: 0.3 mLmin
Column temperature: 40° C.
LC detector: DAD (210 nm)
MSMS: Triple-Quad LCMS (manufactured by Agilent Technologies, Inc.) Ionization method: ESI in negative mode.

The 3-oxoadipyl-CoA reductase activity value can be calculated by quantifying 3-hydroxyadipyl-CoA generated from 3-oxoadipyl-CoA used as a substrate by using purified 3-oxoadipyl-CoA reductase, wherein the 3-oxoadipyl-CoA is prepared from 3-oxoadipic acid by an enzymatic reaction. The specific method is as follows.

3-Oxoadipic acid can be prepared by a known method (for example, a method described in Reference Example 1 of WO 2017099209).

Preparation of 3-oxoadipyl-CoA solution: A PCR using the genomic DNA of Pseudomonas putida strain KT2440 as a template is performed in accordance with routine procedures, to amplify a nucleic acid encoding a CoA transferase (peal and pcaJ; NCBI-GenelDs: 1046613 and 1046612) in the full-length form. The nucleotide sequences of primers used in this PCR are, for example, those represented by SEQ ID NOs: 194 and 195. The amplified fragment is inserted into the Kpnl site of pRSF-1b (manufactured by Novagen), an expression vector for E. coli, in-frame with the histidine-tag sequence. The plasmid is introduced into E. coli BL21 (DE3), and the enzyme is expressed from the plasmid under isopropyl-β-thiogalactopyranoside (IPTG) induction and is then purified using the histidine tag from the culture fluid in accordance with routine procedures to obtain a CoA transferase solution. The solution is used to prepare an enzymatic reaction solution for 3-oxoadipyl-CoA preparation with the following composition, and the enzymatic reaction solution is kept at 25° C. for 3 minutes to allow the reaction to proceed and is then filtered through a UF membrane (Amicon Ultra-0.5mL 10K; manufactured by Merck Millipore) to remove the enzyme, and the obtained filtrate is designated as 3-oxoadipyl-CoA solution.

### (Enzymatic Reaction Solution)

100 mM Tris-HCl (pH 8.2)

10 mM MgCl₂

0.5 mM succinyl-CoA

5 mM 3-oxoadipic acid sodium salt

2 μM CoA transferase.

Identification of 3-oxoadipyl-CoA reductase activity: A PCR using the genomic DNA of a microorganism strain as a template is performed in accordance with routine procedures, to amplify a nucleic acid encoding 3-oxoadipyl-CoA reductase in the full-length form. The nucleotide sequences of primers used in this PCR are, for example, those represented by SEQ ID NOs: 196 and 197. The amplified fragment is inserted into the Ba.mHI site of pACYCDuet-1 (manufactured by Novagen), an expression vector for E. coli, in-frame with the histidine-tag sequence. The plasmid is introduced into E. coli BL21 (DE3), and the enzyme is expressed from the plasmid under isopropyl(3-thiogalactopyranoside (IPTG) induction and is then purified using the histidine tag from the culture fluid in accordance with routine procedures to obtain a 3-oxoadipyl-CoA reductase solution. The 3-oxoadipyl-CoA reductase activity can be determined by using the enzyme solution to prepare an enzymatic reaction solution with the following composition and quantifying 3-hydroxyadipyl-CoA generated at 25° C.

### (Enzymatic Reaction Solution)

100 mM Tris-HCl (pH 8.2)

10 mM MgCl₂

150 μLmL 3-oxoadipyl-CoA solution

0.5 mM NADH

1 mM dithiothreitol

10 μM 3-oxoadipyl-CoA reductase.

In the present invention, the genetically modi lied microorganism in which the expression of any one of the polypeptides described in (a) to (c) is enhanced is a microorganism as a host which originally has a nucleic acid encoding any one of the polypeptides described in (a) to (c) and is genetically modified for increased expression of any one of the polypeptides described in (a) to (c) which are owned by the host microorganism.

Specific examples of the microorganism which originally has a nucleic acid encoding any one of the polypeptides described in (a) to (c) include the following microorganisms of the genus Serratia, including Serratia marcescens (a microorganism having the sequences represented by SEQ ID NOs: 1, 18 to 28, 30 to 33, 35 to 66, 69, 70, 72 to 78, and 79), Serratia nematodiphila (a microorganism having the sequences represented by SEQ ID NOs: 2, 29, and 67), Serratia plymuthica (a microorganism having the sequences represented by SEQ ID NOs: 3, 79 to 84, and 86), Serratia proteamaculans (a microorganism having the sequences represented by SEQ ID NOs: 4 and 85), Serratia ureilytica (a microorganism having the sequence represented by SEQ ID NO: 5), Serratia sp. BW106 (a microorganism having the sequence represented by SEQ ID NO: 6), Serratia liquefaciens (a microorganism having the sequence represented by SEQ ID NO: 7), Serratia sp. S119 (a microorganism having the sequence represented by SEQ ID NO: 8), Serratia sp. YD25 (a microorganism having the sequence represented by SEQ ID NO: 9), Serratia sp. FS14 (a microorganism having the sequence represented by SEQ ID NO: 10). Serratia sp. HMSC15F11 (a microorganism having the sequence represented by SEQ ID NO: I1), Serratia sp. JKS000199 (a microorganism having the sequence represented by SEQ ID NO: 12), Serratia sp. TEL (a microorganism having the sequence represented by SEQ ID NO: 13), Serratia sp. ISTD04 (a microorganism having the sequence represented by SEQ ID NO: 14), Serratia sp. SCBI (a microorganism having the sequence represented by SEQ ID NO: 15), Serratia sp. S4 (a microorganism having the sequence represented by SEQ ID NO: 16), Serratia sp. C-1 (a microorganism having the sequence represented by SEQ ID NO: 17), Serratia sp. OMLW3 (a microorganism having the sequence represented by SEQ ID NO: 34), Serratia sp. OLEL1 (a microorganism having the sequence represented by SEQ ID NO: 68), Serratia sp. OLEL2 (a microorganism having the sequence represented by SEQ ID NO: 71), and the like.

Each of the polypeptides as described above in (a), (b), and (c) also has 3-hydroxybutyryl-CoA dehydrogenase activity, and the 3-hydroxybutyryl-CoA dehydrogenase is encoded by a 3-hydroxybutyryl-CoA dehydrogenase gene, which forms a gene cluster with the 5-aminolevulinic acid synthase gene in the microorganisms of the genus Serratia.

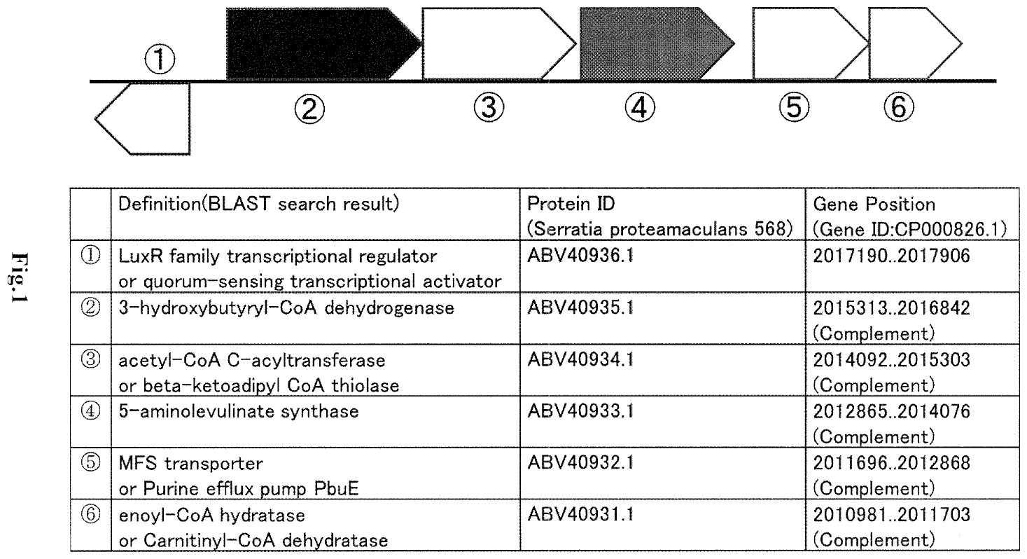

As used herein, the term “gene cluster” in the phrase “the 3-hydroxybutyryl-CoA dehydrogenase gene, which forms a gene cluster with 5-aminolevulinic acid synthase gene in the microorganisms of the genus Serratia” refers to a region in which a set of nucleic acids encoding proteins with related functions are located in close proximity to each other. Specific components in a gene cluster include, for example, nucleic acids which are transcribed under the control of a single transcription regulator, and those in an operon which are transcribed under the control of a single transcription promoter. Whether or not a certain nucleic acid is a nucleic acid component of a gene cluster can also be investigated using an online gene cluster search program, such as antiSMASH. Additionally, whether or not a certain polypeptide is classified as a 3-hydroxybutyryl-CoA dehydrogenase or a 5-aminolevulinic acid synthase can be determined by BLAST (Basic Local Alignment Search Tool) searching on a website, such as that of NCR! (National Center for Biotechnology Information) or KEGG (Kyoto Encyclopedia of Genes and Genomes), to find any enzyme with a high degree of homology to the polypeptide in amino acid sequence. For example, the amino acid sequence represented by SEQ ID NO: 4 is registered in an NCBI database under Protein ID: ABV40935.1, which is annotated as a putative protein with 3-hydroxybutyryl-CoA dehydrogenase activity, as judged from the amino acid sequence. A gene encoding the amino acid sequence represented by SEQ ID NO: 4 is registered in an NCBI database under Gene ID: CP000826.1 and can be identified through a database search as conserved in the genome of Serratia proteamaculans strain 568 or as conserved in the region from 2015313 to 2016842 bp on the sequence of Gene ID: CP000826.1. Furthermore, the positional information of the gene can lead to identification of the sequences of flanking genes, from which the gene can be found to form a gene cluster with the 5-aminolevulinic acid synthase gene (Protein ID: ABV40933.1), as shown in FIG. 1. Similarly, for the amino acid sequences represented by SEQ ID NOs: 1 to 3, 6 to 20, 22 to 30,32 to 35,37,38,40,42 to 48,51 to 56, 59 to 63, 65, 66, 68 to 73, 75 to 81, and 83 to 85, the information can be checked on the NCBI site with the Protein IDs and Gene IDs presented in Tables 3-4 and 3-5.

TABLE 3-4

| SEQ | | |
|---|---|---|
| ID | | |
| NO: | Gene ID:position (from . . . to) | Protein ID |
| 1 | JMPQ01000047.1:133194 . . . 134723 | KFD11732.1 |
| 2 | JPUX00000000.1:4202615 . . . 4204144 | WP_033633399.1 |
| 3 | BCTU01000013.1:85647 . . . 87176 | WP_063199278.1 |
| 4 | CP000826.1:2015313 . . . 2016842 | ABV40935.1 |
| 6 | MCGS01000002.1:43811 . . . 45340 | WP_099061672.1 |
| 7 | CP006252.1:1825868 . . . 1827397 | AGQ30498.1 |
| 8 | MSFH01000022.1:147976 . . . 149505 | ONK16968.1 |
| 9 | CP016948.1:1213474 . . . 1215003 | AOE98783.1 |
| 10 | CP005927.1:4244665 . . . 4246194 | WP_044031504.1 |
| 11 | LWNG01000196.1:83086 . . . 84615 | OFS85208.1 |
| 12 | LT907843.1:1172733 . . . 1174262 | SNY82966.1 |
| 13 | LDEG01000005.1:19627 . . . 21156 | KLE40298.1 |
| 14 | MBDW01000089.1:53478 . . . 55007 | ODJ15373.1 |
| 15 | CP003424.1:1869825 . . . 1871300 | AIM21329.1 |
| 16 | APLA01000003.1:1964823 . . . 1966352 | WP_017892361.1 |
| 17 | CAQO01000118.1:101692 . . . 103221 | WP_062792820.1 |
| 18 | JVDI01000070.1:19399 . . . 20928 | WP_049300487.1 |
| 19 | FCGF01000001.1:938090 . . . 939619 | WP_060444298.1 |
| 20 | NC_020211.1:1963542 . . . 1965071 | WP_015377392.1 |
| 22 | JVNC01000043.1:47711 . . . 49240 | WP_049187553.1 |
| 23 | MCNK01000010.1:591271 . . . 592800 | WP_076740355.1 |
| 24 | JVZV01000138.1:53080 . . . 54609 | WP_049277247.1 |
| 25 | CP021984.1:1963542 . . . 1965071 | WP_088381461.1 |
| 26 | NERL01000025.1:86571 . . . 88100 | WP_060559176.1 |
| 27 | MTEH01000001.1:215863 . . . 217392 | WP_085336366.1 |
| 28 | JVCS01000001.1:19397 . . . 20926 | WP_049239700.1 |
| 29 | MTBJ01000002.1:216232 . . . 217761 | WP_082996863.1 |
| 30 | AORJ01000010.1:70272 . . . 71801 | WP_033645451.1 |
| 32 | LFJS01000012.1:944087 . . . 945616 | WP_025302345.1 |
| 33 | CP018930.1:1161338 . . . 1162867 | WP_060447438.1 |
| 34 | MSTK01000013.1:54046 . . . 55575 | WP_099817374.1 |
| 35 | CP018929.1:1167577 . . . 1170106 | WP_089180755.1 |
| 37 | FCGS01000006.1:98915 . . . 100444 | WP_060438851.1 |
| 38 | MQRI01000002.1:585500 . . . 587029 | WP_060387554.1 |
| 40 | FCFE01000001.1:962839 . . . 964368 | WP_060435888.1 |
| 42 | FCIO01000002.1:145369146898 . . . | WP_033637938.1 |
| 43 | AP013063.1:1329259 . . . 1330788 | WP_041034581.1 |
| 44 | MQRJ01000004.1:178926 . . . 180455 | WP_074026553.1 |
| 45 | HG738868.1:1928329 . . . 1929858 | WP_060437960.1 |

TABLE 3-5

| SEQ | | |
|---|---|---|
| ID | | |
| NO: | Gene ID:position (from . . . to) | Protein ID |
| 46 | FCHQ01000006.1:51377 . . . 52906 | WP_060420535.1 |
| 47 | NPGG01000001.1:301231 . . . 302760 | WP_047568134.1 |
| 48 | FCME01000002.1:205632 . . . 207161 | WP_060443161.1 |
| 51 | FCIH01000014.1:52403 . . . 53932 | WP_060429049.1 |
| 52 | NBWV01000007.1:110621 . . . 112150 | WP_039566649.1 |
| 53 | FCKI01000001.1:594106 . . . 595635 | WP_060429902.1 |
| 54 | JPOB01000010.1:81351 . . . 82880 | WP_033654196.1 |
| 55 | FCFI01000001.1:582222 . . . 583751 | WP_060443342.1 |
| 56 | FCML01000001.1:1005802 . . . 1007331 | WP_060456892.1 |
| 59 | FCMR01000001.1:1873566 . . . 1875095 | WP_060440240.1 |
| 60 | LJEV02000002.1:115432 . . . 116961 | WP_047727865.1 |
| 61 | NPIX01000027.1:38249 . . . 39778 | WP_094461128.1 |
| 62 | NDXU01000091.1:70343 . . . 71872 | WP_048233299.1 |
| 63 | FNXW01000055.1:13619 . . . 15148 | WP_080490898.1 |
| 65 | AYMO01000023.1:23978 . . . 25507 | WP_025160335.1 |
| 66 | CP018926.1:1215941 . . . 1217470 | WP_089191486.1 |
| 68 | MORG01000026.1:13723 . . . 15252 | WP_099782744.1 |
| 69 | PEHC01000008.1:57274 . . . 58803 | PHY81681.1 |
| 70 | MEDA01000063.1:13491 . . . 15020 | WP_072627918.1 |
| 71 | MORH01000030.1:13633 . . . 15162 | WP_099789708.1 |
| 72 | KK214286.1:392757 . . . 394286 | WP_033650708.1 |
| 73 | KI929259.1:1574567 . . . 1576096 | WP_033642621.1 |
| 75 | CP012639.1:230596 . . . 232125 | WP_060659686.1 |
| 76 | LZOB01000011.1:1613417 . . . 1614946 | WP_074054551.1 |
| 77 | LCWI01000024.1:46336 . . . 47865 | WP_046899223.1 |
| 78 | CP018924.1:1213305 . . . 1214834 | WP_089194521.1 |
| 79 | NC_015567.1:1930552 . . . 1932081 | WP_013812379.1 |
| 80 | MQML01000205.1:9362 . . . 10891 | WP_073439751.1 |
| 81 | AMSV01000032.1:251478 . . . 253007 | WP_006324610.1 |
| 83 | CP007439.1:1991332 . . . 1992861 | AHY06789.1 |
| 84 | CP012096.1:319897 . . . 321426 | WP_037432641.1 |
| 85 | FWWG01000018.1:38528 . . . 40057 | WP_085116175.1 |

A nucleic acid encoding a polypeptide encoded by the 3-hydroxybutyryl-CoA dehydrogenase gene of a microorganism of the genus Serratia , which forms a gene cluster with the 5-aminolevulinic acid synthase gene, is hereinafter referred to as “the 3-hydroxybutyryl-CoA dehydrogenase gene used in the present invention,” and the polypeptide encoded by the 3-hydroxybutyryl-CoA dehydrogenase gene is referred as “the 3-hydroxybutyryl-CoA dehydrogenase used in the present invention.”

A gene cluster including the 3-hydroxybutyryl-CoA dehydrogenase gene used in the present invention may include other nucleic acids, provided that the gene cluster includes at least the 3-hydroxybutyryl-CoA dehydrogenase gene and the 5-aminolevulinic acid synthase gene. FIG. 1 shows a specific example of the gene cluster including the 3-hydroxybutyryl-CoA dehydrogenase gene used in the present invention.

Specific examples of the microorganisms of the genus Serratia that contain the above gene cluster include S. marcescens, S. nematodiphila, S. plymuthica, S. proteamaculans, S. ureilytica, S. liquelaciens. Serratia sp. BW106, Serratia sp. S119, Serratia sp. YD25, Serratia sp. FS14. Serratia sp. HMSC15F11, Serratia sp. JKS000199, Serratia sp. TEL, Serratia sp. ISTD04, Serratia sp. SCBI, Serratia sp. S4, Serratia sp. C-1, Serratia sp. OMLW3. Serratia sp. OLEL1, Serratia sp. OLEL2, and S. liquefaciens.

The 3-hydroxybutyryl-CoA dehydrogenase used in the present invention has an excellent 3-oxoadipyl-CoA reductase activity. Whether or not a 3-hydroxybutyryl-CoA dehydrogenase-encoding nucleic acid has a 3-oxoadipyl-CoA reductase activity can be determined by the same method as described above.

The polypeptide encoded by the 3-hydroxybutyryl-CoA dehydrogenase gene used in the present invention is characterized by containing the common sequence 1. Specific examples of amino acid sequences of such polypeptides include the amino acid sequences represented by SEQ ID NOs: 1 to 86.

In the present invention, a nucleic acid encoding a polypeptide composed of the same amino acid sequence as that represented by any one of SEQ ID NOs: 8 to 86, except that one or several amino acids are substituted, deleted, inserted, andor added, and having an enzymatic activity that catalyzes a reaction to reduce 3-oxoadipyl-CoA to 3-hydroxyadipyl-CoA can also be suitable for use, provided that the common sequence 1 is contained in the polypeptide. In this respect, the range represented by the phrase “gone or several” is preferably 10 or less, more preferably 5 or less, especially preferably 4 or less, and most preferably one or two. In the case of amino acid substitution, the activity of the original polypeptide is more likely to be maintained when an amino acid(s) isare replaced by an amino acid(s) with similar properties (i.e., conservative substitution as described above). A nucleic acid encoding a polypeptide composed of an amino acid sequence with a sequence identity to not less than 70%, preferably not less than 80%, more preferably not less than 85%, further preferably not less than 90%, still further preferably not less than 95%, yet further preferably not less than 97%, even further preferably not less than 99%, to the sequence represented by any one of SEQ ID NOs: 8 to 86 and having an enzymatic activity that catalyzes a reaction to reduce 3-oxoadipyl-CoA to 3-hydroxyadipyl-CoA can also be suitably used.

On the other hand, examples of a polypeptide that is not the 3-hydroxybutyryl-CoA dehydrogenase used in the present invention but has 3-oxoadipyl-CoA reductase activity include PaaH from Pseudomonas putida strain KT2440 (SEQ ID NO: 178), PaaH from Escherichia coli strain K-12 substrain MG1655 (SEQ ID NO: 179). DcaH from Acinetobacter haylyi strain ADP1 (SEQ ID NO: 180), and PaaH from Serratia plymuthica strain NBRC102599 (SEQ ID NO: 181). As shown in Tables 4 and 5, these polypeptides are found not to contain the common sequence I. It should be noted that those polypeptides are neither (b) polypeptides composed of the same amino acid sequence as that represented by any one of SEQ ID NOs: 1 to 7, except that one or several amino acids are substituted, deleted, inserted, andor added, and having an enzymatic activity that catalyzes a reaction to reduce 3-oxoadipyl-CoA to 3-hydroxyadipyl-CoA, nor (c) polypeptides having an amino acid sequence with a sequence identity of not less than 70% to the sequence represented by any one of SEQ ID NOs: 1 to 7 and having an enzymatic activity that catalyzes a reaction to reduce 3-oxoadipyl-CoA to 3-hydroxyadipyl-CoA.

TABLE 4

| | | | 5 10 15 20 25 30 35 40 |
|---|---|---|---|
| | | | Consensus sequence1 |
| | | | GAGTMGRG\|AYLXAXXX\|XTXLYN |
| 1. | 1 | Serratia marcescens ATCC13880 | MAESNAA\|QSAAI\|GAGTMGRG\|AYLFAQKG\|RTVLYNRN |
| 2. | 2 | Serratia nematodiphila DSM21420 | MAESNAA\|QSAAI\|GAGTMGRG\|AYLFAQKG\|PTMLYNRN |
| 3. | 3 | Serratia plymuthica NBRC102599 | MAENNSA\|RSAAV\|GAGTMGRG\|AYLLALNG\|RTVLYNRN |
| 4. | 4 | Serratia proteamaculans 568 | MAENNSA\|HSVAV\|GAGTMGRG\|AYLLAQNG\|RTLLYNRS |
| 5. | 5 | Serratia ureilytica Lr5/4 | MAESNAA\|QSAAI\|GAGTMGRG\|AYLLAQKS\|RTVLYNRN |
| 6. | 6 | Serratia sp. BW106 | MAENNSA\|HSVAV\|GAGTMGRG\|AYLLAQNG\|RTLLYNRS |
| 7. | 7 | Serratia liquefaciens FK01 | MAENNTA\|DSVAV\|GAGTMGRG\|AYLLALNG\|RTLLYNRN |
| 8. | 8 | Serratia sp. S119 | MAESNAA\|QSAAI\|GAGTMGRG\|AYLFAQKG\|RTVLYNRN |
| 9. | 9 | Serratia sp. YD25 | MAERNAA\|QSAAI\|GAGTMGRG\|AYLFAQKG\|RTVLYNRN |
| 10. | 10 | Serratia sp. FS14 | MAESNAA\|QSAAI\|GAGTMGRG\|AYLFAQKG\|PTMLYNRN |
| 11. | 11 | Serratia sp. HMSC15F11 | MAESNAA\|QSAAI\|GAGTMGRG\|AYLFAQKG\|RTVLYNRN |
| 12. | 12 | Serratia sp. JKS000199 | MAESNAA\|QSAAI\|GAGTMGRG\|AYLLAQKS\|RTVLYNRN |
| 13. | 13 | Serratia sp. TEL | MAESNAA\|QSAAI\|GAGTMGRG\|AYLLAQKS\|RTVLYNRN |
| 14. | 14 | Serratia sp. ISTD04 | MAESNAA\|QSAAI\|GAGTMGRG\|AYLLAQKS\|RTVLYNRN |
| 15. | 15 | Serratia sp. SCBI | MAESNAA\|QSAAI\|GAGTMGRG\|AYLLAQKS\|RTVLYNRN |
| 16. | 16 | Serratia sp. S4 | MAENNSA\|HSVAV\|GAGTMGRG\|AYLLAQNG\|RTLLYNRS |
| 17. | 17 | Serratia sp. C-1 | MAENNSA\|RSAAV\|GAGTMGRG\|AYLLALNG\|RTVLYNRN |
| 18. | 18 | Serratia marcescens 532 | MAESNAA\|QSAAI\|GAGTMGRG\|AYLFAQKG\|RTVLYNRN |
| 19. | 19 | Serratia marcescens 2880STDY5683033 | MAESNAA\|QSAAI\|GAGTMGRG\|AYLFAQKG\|RTVLYNRN |
| 20. | 20 | Serratia marcescens WW4 | MAESNAA\|QSAAI\|GAGTMGRG\|AYLFAQKG\|PTMLYNRN |
| 21. | 21 | Serratia marcescens K27 | MAESNAA\|QSAAI\|GAGTMGRG\|AYLFAQKG\|PTMLYNRN |
| 22. | 22 | Serratia marcescens 280 | MAESNAA\|QSAAI\|GAGTMGRG\|AYLFAQKG\|PTMLYNRN |
| 23. | 23 | Serratia marcescens 19F | MAESNAA\|QSAAI\|GAGTMGRG\|AYLFAQKG\|PTMLYNRN |
| 24. | 24 | Serratia marcescens 1185 | MAESNAA\|QSAAI\|GAGTMGRG\|AYLFAQKG\|PTMLYNRN |
| 25. | 25 | Serratia marcescens S217 | MAESNAA\|QSAAI\|GAGTMGRG\|ATLFAQKG\|PTMLYNRN |
| 26. | 26 | Serratia marcescens KHCo-24B | MAESNAA\|QSAAI\|GAGTMGRG\|AYLFAQKG\|PTMLYNRN |
| 27. | 27 | Serratia marcescens Z6 | MAESNAA\|QSAAI\|GAGTMGRG\|AYLFAQKG\|PTMLYNRN |
| 28. | 28 | Serratia marcescens 546 | MAESNAA\|QSAAI\|GAGTMGRG\|AYLFAQKG\|PTMLYNRN |
| 29. | 29 | Serratia nematodiphila MB307 | MAESNAA\|QSAAI\|GAGTMGRG\|AYLFAQKG\|PTMLYNRN |
| 30. | 30 | Serratia marcescens VGH107 | MAESNAA\|QSAAI\|GAGTMGRG\|AYLFAQKG\|PTMLYNRN |
| 31. | 31 | Serratia marcescens MCB | MAESNAA\|QSAAI\|GAGTMGRG\|AYLFAQKG\|RTVLYNRN |
| 32. | 32 | Serratia marcescens AH0650 | MAESNAA\|QSAAI\|GAGTMGRG\|AYLFAQKG\|RTVLYNRN |
| 33. | 33 | Serratia marcescens UMH12 | MAESNAE\|QSAAI\|GAGTMGRG\|AYLFAQKG\|RTVLYNRN |
| 34. | 34 | Serratia sp. OMLW3 | MAESNAA\|QSAAI\|GAGTMGRG\|AYLFAQKG\|RTVLYNRN |
| 35. | 35 | Serratia marcescens UMH11 | MAESNAA\|QSAAI\|GAGTMGRG\|AYLFAQKG\|RTVLYNRS |
| 36. | 36 | Serratia marcescens UMH1 | MAESNAA\|QSAAI\|GAGTMGRG\|AYLFAQKG\|RTVLYNRN |
| 37. | 37 | Serratia marcescens 2880STDY5683020 | MAESNAA\|QSAAI\|GAGTMGRG\|AYLFAQKG\|RTVLYNRN |
| 38. | 38 | Serratia marcescens 99 | MAESNAA\|QSAAI\|GAGTMGRG\|AYLFAQKG\|RTVLYNRN |
| 39. | 39 | Serratia marcescens 374 | MAESNAA\|QSAAI\|GAGTMGRG\|AYLFAQKG\|RTVLYNRN |
| 40. | 40 | Serratia marcescens 2880STDY5683036 | MAESNAA\|QSAAI\|GAGTMGRG\|AYLFAQKG\|RTVLYNRN |
| 41. | 41 | Serratia marcescens 2880STDY5683034 | MAESNAA\|QSAAI\|GAGTMGRG\|AYLFAQKG\|RTVLYNRN |
| 42. | 42 | Serratia marcescens 2880STDY5682892 | MAESNAA\|QSAAI\|GAGTMGRG\|AYLFAQKG\|RTVLYNRN |
| 43. | 43 | Serratia marcescens SM39 | MAESNAA\|QSAAI\|GAGTMGRG\|AYLFAQKG\|RTVLYNRN |

TABLE 5

| | | | 5 10 15 20 25 30 35 40 |
|---|---|---|---|
| | | | Consensus sequence1 |
| | | | GAGTMGRG\|AYLXAXXX\|XTXLYN |
| 44. | 44 | Serratia marcescens 189 | MAESNAA\|QSAAI\|GAGTMGRG\|AYLFAQKG\|RTVLYNRN |
| 45. | 45 | Serratia marcescens SMB2099 | MAESNAA\|QSAAI\|GAGTMGRG\|AYLFAQKG\|RTVLYNRN |
| 46. | 46 | Serratia marcescens 2880STDY5682862 | MAESNAA\|QSAAI\|GAGTMGRG\|AYLFAQKG\|RTVLYNRN |
| 47. | 47 | Serratia marcescens SE4145 | MAESNAE\|QSAAI\|GAGTMGRG\|AYLFAQKG\|RTVLYNRN |
| 48. | 48 | Serratia marcescens 2880STDY5682876 | MAESNAA\|QSAAI\|GAGTMGRG\|AYLFAQKG\|RTVLYNRN |
| 49. | 49 | Serratia marcescens 709 | MAESNAA\|QSAAI\|GAGTMGRG\|AYLFAQKG\|RTVLYNRN |
| 50. | 50 | Serratia marcescens MGH136 | MAESNAA\|QSAAI\|GAGTMGRG\|AYLFAQKG\|RTVLYNRN |
| 51. | 51 | Serratia marcescens 2880STDY5682884 | MAESNAA\|QSAAI\|GAGTMGRG\|AYLFAQKG\|RTVLYNRN |
| 52. | 52 | Serratia marcescens D-3 | MAESNAA\|QSAAI\|GAGTMGRG\|AYLFAQKG\|RTVLYNRN |
| 53. | 53 | Serratia marcescens 2880STDY5682957 | MAESNAA\|QSAAI\|GAGTMGRG\|AYLFAQKG\|RTVLYNRN |
| 54. | 54 | Serratia marcescens YDC563 | MAESNAA\|QSAAI\|GAGTMGRG\|AYLFAQKG\|RTVLYNRN |
| 55. | 55 | Serratia marcescens 2880STDY5683035 | MAESNAA\|QSAAI\|GAGTMGRG\|AYLFAQKG\|RTVLYNRN |
| 56. | 56 | Serratia marcescens 2880STDY5682930 | MAESNAA\|QSAAI\|GAGTMGRG\|AYLFAQKG\|RTVLYNRN |
| 57. | 57 | Serratia marcescens 790 | MAESNAA\|QSAAI\|GAGTMGRG\|AYLFAQKG\|RTVLYNRN |
| 58. | 58 | Serratia marcescens UMH5 | MAESNAA\|QSAAI\|GAGTMGRG\|AYLFAQKG\|RTVLYNRN |
| 59. | 59 | Serratia marcescens 2880STDY5682988 | MAESNAA\|QSAAI\|GAGTMGRG\|AYLFAQKG\|RTVLYNRN |
| 60. | 60 | Serratia marcescens 945154301 | MAESNAA\|QSAAI\|GAGTMGRG\|AYLFAQKG\|RTVLYNRN |
| 61. | 61 | Serratia marcescens at10508 | MAESNAA\|QSAAI\|GAGTMGRG\|AYLFAQKG\|RTVLYNRN |
| 62. | 62 | Serratia marcescens ML2637 | MAESNAA\|QSAAI\|GAGTMGRG\|AYLFAQKG\|RTVLYNRN |
| 63. | 63 | Serratia marcescens SM1978 | MAESNAA\|QSAAI\|GAGTMGRG\|AYLFAQKG\|RTVLYNRN |
| 64. | 64 | Serratia marcescens PWN146 | MAESNAA\|QSAAI\|GAGTMGRG\|AYLFAQKG\|RTVLYNRN |
| 65. | 65 | Serratia marcescens H1q | MAERNAA\|QSAAI\|GAGTMGRG\|AYLFAQKG\|RTVLYNRN |
| 66. | 66 | Serratia marcescens UMH6 | MAESNAA\|QSAAI\|GAGTMGRG\|AYLFAQKS\|RTVLYNRN |
| 67. | 67 | Serratia nematodiphila WCU338 | MAESNAA\|QSAAI\|GAGTMGRG\|AYLFAQKS\|RTVLYNRN |
| 68. | 68 | Serratia sp. OLEL1 | MAESNAA\|QSAAI\|GAGTMGRG\|AYLLAQKS\|RTVLYNRN |
| 69. | 69 | Serratia marcescens 7209 | MAESNAA\|QSAAI\|GAGTMGRG\|AYLFAQKS\|RTVLYNRN |
| 70. | 70 | Serratia marcescens sicaria (Ss1) | MAESNAA\|QSAAI\|GAGTMGRG\|AYLFAQKS\|RTVLYNRN |
| 71. | 71 | Serratia sp. OLFL2 | MAESNAA\|QSAAI\|GAGTMGRG\|AYLLAQKS\|RTVLYNRN |
| 72. | 72 | Serratia marcescens BIDMC 81 | MAESNAA\|QSAAI\|GAGTMGRG\|AYLLAQKS\|RTVLYNRN |
| 73. | 73 | Serratia marcescens BIDMC 50 | MAESNAA\|QSAAI\|GAGTMGRG\|AYLFAQKS\|RTVLYNRN |
| 74. | 74 | Serratia marcescens UMH7 | MAESNAA\|QSAAI\|GAGTMGRG\|AYLLAQKS\|RTVLYNRN |
| 75. | 75 | Serratia marcescens RSC-14 | MAESNAA\|QSAAI\|GAGTMGRG\|AYLLAQKS\|RTVLYNRN |
| 76. | 76 | Serratia marcescens SMO3 | MAERNAA\|QSAAI\|GAGTMGRG\|AYLFAQKG\|RTVLYNRN |
| 77. | 77 | Serratia marcescens 90-166 | MAESNAA\|QSAAI\|GAGTMGRG\|AYLFAQKG\|RTVLYNRN |
| 78. | 78 | Serratia marcescens UMH2 | MAESNAA\|QSAAI\|GAGTMGRG\|AYLLAQKS\|RTVLYNRN |
| 79. | 79 | Serratia plymuthica AS9 | MAENNSA\|RSAAV\|GAGTMGRG\|AYLLALNG\|RTVLYNRN |
| 80. | 80 | Serratia plymuthica tumat 205 | MAENNSA\|RSAAV\|GAGTMGRG\|AYLLALNG\|RTVLYNRN |
| 81. | 81 | Serratia plymuthica A30 | MAENNSA\|RSAAV\|GAGTMGRG\|AYLLALNG\|RTVLYNRN |
| 82. | 82 | Serratia plymuthica 4Rx13 | MAENNSA\|RSAAV\|GAGTMGRG\|AYLLALNG\|RTVLYNRN |
| 83. | 83 | Serratia plymuthica V4 | MAENNSA\|RSAAV\|GAGTMGRG\|AYLLALNG\|RTVLYNRN |
| 84. | 84 | Serratia plymuthica 3Rp8 | MAENNSA\|RSAAV\|GAGTMGRG\|AYLLALNG\|RTVLYNRN |
| 85. | 85 | Serratia proteamaculans MFPA44A14 | MAENNSA\|HSVAV\|GAGTMGRG\|AYLLAQNG\|RTLLYNRS |
| 86. | 86 | Serratia plymuthica A153 | MAENNSA\|RSAAV\|GAGTMGRG\|AYLLALNG\|RTVLYNRN |

In the present invention, impairing the function of pyruvate kinase or a phosphotransferase system enzyme means impairing the enzymatic activity of the enzyme. The method of impairment of the function is not limited to a particular method, but the function can be impaired, for example, by disrupting a gene that encodes the enzyme, such as via partial or complete deletion of the gene by mutagenesis with a chemical mutagen, ultraviolet irradiation, or the like, or by site-directed mutagenesis or the like, or via introduction of a frame-shift mutation or a stop codon into the nucleotide sequence of the gene. Alternatively, recombinant DNA technologies can be used to disrupt the gene by partial or complete deletion of the nucleotide sequence or by partial or complete substitution of the nucleotide sequence with another nucleotide sequence. Among those, the methods for partial or complete deletion of the nucleotide sequence are preferred.

Pyruvate kinase is classified as EC 2.7.1.40 and is an enzyme that catalyzes a reaction to dephosphorylate phosphoenolpyruvic acid (in this specification, also referred to as PEP) to pyruvic acid and ATP. Specific examples of pyruvate kinase include pykF (NCBI-Protein ID: NP 416191, SEQ ID NO: 182) and pykA (NCBI-Protein ID: NP 416368, SEQ ID NO: 183) from Escherichia coli strain K-12 substrain MG1655, and pykF (SEQ ID NO: 184) and pykA (SEQ ID NO: 185) from Serratia grimesii strain NBRC13537.

In cases where a microorganism used in the present invention has two or more genes that each encode a pyruvate kinase, as illustrated in the metabolic pathway shown in the scheme 2 below, it is desirable to impair the function of all the pyruvate kinases. Whether or not a polypeptide encoded by a certain gene of a microorganism used in the present invention is a pyruvate kinase may be determined by BLAST (Basic Local Alignment Search Tool) searching on a website, such as that of NCBI (National Center for Biotechnology Information) or KEGG (Kyoto Encyclopedia of Genes and Genomes).

In the genetically modified microorganism of the present invention, it is desirable to further impair the function of a phosphotransferase system enzyme. The phosphotransferase system enzyme is relevant to the phosphoenolpyruvate

(PEP)-dependent phosphotransferase system (PTS) (in this specification, also referred to as a PTS enzyme). PTS is a major mechanism for the uptake of carbohydrates such as hexose, hexitol, and disaccharide into a cell, as illustrated in the metabolic pathway shown in the scheme 2 below. PTS involves uptake of carbohydrates into a cell and simultaneous conversion of the carbohydrates to a phosphate ester, while converting a phosphate donor, PEP, to pyruvic acid. Therefore, the conversion reaction from PEP to pyruvic acid is inhibited in a mutant microorganism with a disrupted PTS enzyme gene.

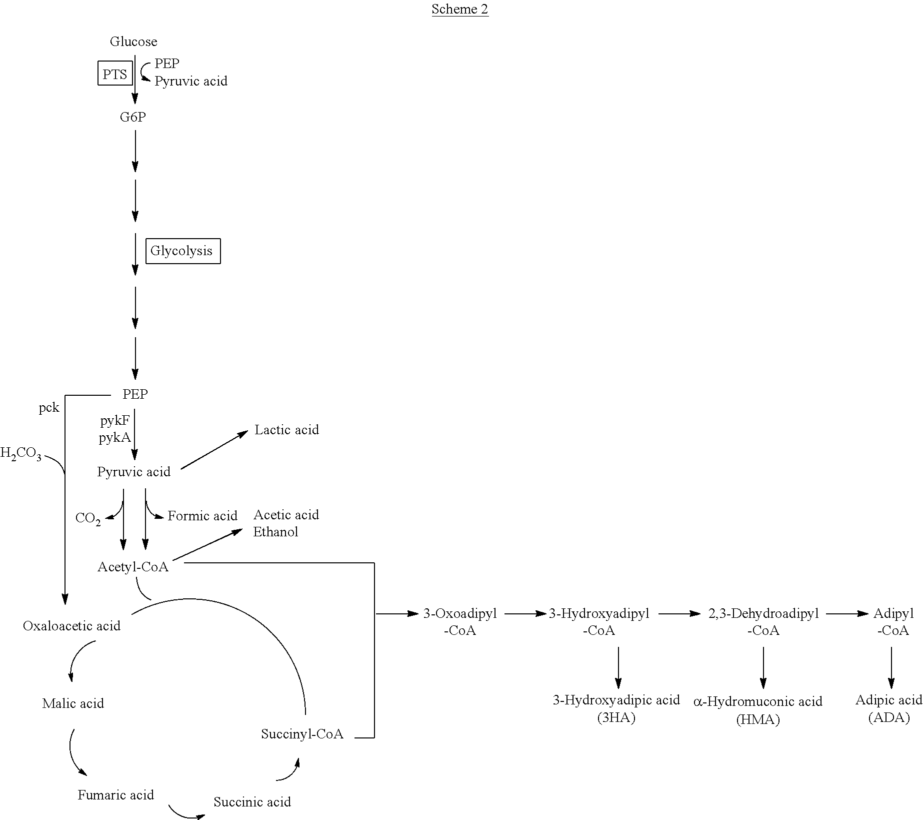

PTS enzymes are composed of two common enzymes that exert their functions on any type of carbohydrate, phosphoenolpyruvate sugar phosphotransferase enzyme I and phospho carrier protein HPr, and membrane-bound sugar specific permeases (enzymes II) that are specific for particular carbohydrates. The enzymes II are further composed of sugar-specific components IIA, IIB, and IIC. The enzymes II exist as independent proteins or as fused domains in a single protein, and this depends on the organism which those enzymes are originated from. In microorganisms, phosphoenolpyruvate sugar phosphotransferase enzyme I is encoded by the pts gene, and phospho carrier protein 1-1Pr is encoded by the ptsH gene, and glucose-specific enzyme HA is encoded by the crr gene, and glucose-specific enzymes IIB and HC are encoded by the ptsG gene. The enzyme encoded by the ptsG gene is classified as EC 2.7.1.199 and is called protein-Npi-phosphohistidinc-D-glucose phosphotransferase.

In the present invention, one or more of the above PTS enzyme genes may be disrupted. Although any of the above PTS enzyme genes may be disrupted, it is desirable to impair an enzyme gene that is involved in glucose uptake, particularly the ptsG gene. Specific examples of the ptsG gene include ptsG from Escherichia coli strain K-12 substrain MG1655 (NCBI-Gene ID: 945651) andptsG from Serratia grimesii strain NBRC13537 (SEQ ID NO: 238).

Whether or not a polypeptide encoded by a certain gene of a microorganism used in the present invention is a protein-Npi-phosphohistidine-D-glucose phosphotransferase may be determined by BLAST searching on a website, such as that of NCBI or KEGG.

As described below, E. coli is a microorganism that has an ability to produce 3-hydroxyadipic acid and α-hydromuconic acid, and JP 2008-527991 A describes production of a genetically modified E. coli strain with defects in the pykF and pykA genes, which each encode a pyruvate kinase, and in the ptsG gene, which encodes a phosphotransferase system enzyme, wherein the yield of succinic acid is increased, and the yields of acetic acid and ethanol are decreased, by culturing the genetically modified strain under anaerobic conditions. In this respect, acetic acid and ethanol are compounds generated from the metabolism of acetyl-CoA, as illustrated in the metabolic pathway shown in the above scheme 2. That is, in JP 2008-527991 A. it is presumed that the defects of the ptsG, pykF, and pykA genes in E. coli resulted in a reduced supply of acetyl-CoA and in turn a lower yield of acetic acid and ethanol.

The 3-hydroxyadipic acid, α-hydromuconic acid, andor adipic acid produced by the method of the present invention are compounds generated through reactions in the metabolism of 3-oxoadipyl-CoA, which is produced from acetyl-CoA and succinyl-CoA by the reaction A, as described above. Accordingly, from the description in JP 2008-527991 A, it is expected that disruption of genes encoding pyruvate kinase and a phosphotransferase system enzyme also results in a decreased yields of 3-hydroxyadipic acid, α-hydromuconic acid, andor adipic acid due to the reduced supply of acetyl-CoA. However, in the present invention, disruption of genes encoding pyruvate kinase and a phosphotransferase system enzyme increases the yields of 3-hydroxyadipic acid, α-hydromuconic acid, andor adipic acid and also the yields of acetic acid and ethanol in a genetically modified microorganism expressing an enzyme that exhibits excellent activity in a reaction to reduce 3-oxoadipyl-CoA to 3-hydroxyadipyl-CoA, which is contrary to the above expectation.

In the present invention, examples of the microorganism that can be used as a host to obtain the genetically modified microorganism include microorganisms belonging to the genera Escherichia, Serratia, Hafnia, Pseudomonas, Corynebacterium, Bacillus. Streptomyces, Cupriavidus, Acinetobacter, Alcaligenes, Brevibacterium, Delftia, Aerobacter, Rhizobium, ThermoNfida, Clostridium, Schizosaccharomyces, Kluyveromyees, Pichia, and Candida. Among those, microorganisms belonging to the genera Escherichia, Serratia, Hafnia, and Pseudomonas are preferred.

The method of producing 3-hydroxyadipic acid, α-hydromuconic acid, andor adipic acid by using a genetically modified microorganism of the present invention will be described.

As a microorganism that has an ability to produce 3-hydroxyadipic acid, a microorganism that has an ability to generate 3-oxoadipyl-CoA and coenzyme A from acetyl-CoA and succinyl-CoA (the reaction A), and an ability to generate 3-hydroxyadipic acid from 3-hydroxyadipyl-CoA (the reaction E) is used. The microorganism with these production abilities can be used as a host microorganism to obtain a genetically modified microorganism according to the present invention with an ability to abundantly produce 3-hydroxyadipic acid.

Microorganisms that are speculated to originally have abilities to catalyze the above reactions A and E include microorganisms belonging to the following species:

species of the genus Escherichia, such as Escherichia fergusonii and Escherichia coli;

species of the genus Pseudomonas, such as Pseudomonas chlororaphis, Pseudomonas putida, Pseudomonas azotoformans, and Pseudomonas chlororaphis subsp. aureofaciens;

species of the genus Hafnia, such as Hafnia alvei;

species of the genus Corynebacterium, such as Corynebacterium acetoacidophilum, Corynebacterium acetoglulamicum, Corynebacterium ammoniagenes, and Corynebacterium glutamicum;

species of the genus Bacillus, such as Bacillus badius, Bacillus magaterium, and Bacillus roseus;

species of the genus Streptomyces, such as Streptomyces vinaceus, Streptomyces karnatakensis, and Streptomyces olivaceus;

species of the genus Cupriavidus, such as Cupriavidus metallidurans, Cupriavidus necator, and Cupriavidus oxalaticus;

species of the genus Acinetobacter, such as Acinetobacter baylyi and Acinetobacter radioresistens;

species of the genus Alcaligenes, such as Alcaligenes faecalis;

species of the genus Nocardioides, such as Nocardioides albus;

species of the genus Brevibacterium, such as Brevibacterium iodinum;

species of the genus Delfila, such as Delftia acidovorans;

species of the genus Shimwellia, such as Shimwellia blattae;

species of the genus Aerobacter, such as Aerobacter cloacae;

species of the genus Rhizobium, such as Rhizobium radiobacter;

species of the genus Serratia, such as Serratia grimesii, Serratia ficaria, Serratia fonticokt, Serratia odorfera, Serratia plymuthica, Serratia entomophila, and Serratia nematodiphila.

Even a microorganism that originally has no abilities to catalyze the reactions A andor E can also be used as the aforementioned host microorganism when an appropriate combination of nucleic acids that encode enzymes catalyzing the reactions A and E is introduced into the microorganism to impart those production abilities.

As a microorganism that has an ability to produce α-hydromuconic acid, a microorganism that has an ability to generate 3-oxoadipyl-CoA and coenzyme A from acetyl-CoA and succinyl-CoA (the reaction A), an ability to generate 2,3-dehydroadipyl-CoA from 3-hydroxyadipyl-CoA through dehydration (the reaction C), and an ability to generate α-hydromuconic acid from 2.3-dehydroadipyl-CoA (the reaction F) is used. The microorganism with these production abilities can be used as a host microorganism to obtain a genetically modified microorganism according to the present invention with an ability to abundantly produce α-hydromuconic acid.

Microorganisms that are speculated to originally have abilities to catalyze the above reactions A, C, and F include microorganisms belonging to the following species:

species of the genus Escherichia, such as Escherichia jergusonii and Escherichia coli;

species of the genus Pseudomonas, such as Pseudomonas fluorescens, Pseudomonas putida, Pseudomonas azotoformans, and Pseudomonas chlororaphis subsp. aureofaciens;

species of the genus Hafnia, such as Hafnia alvei;

species of the genus Bacillus, such as Bacillus badius;

species of the genus Cupriavidus, such as Cupriavidus metallidurans, Cupriavidus numazuensis, and Cupriavidus oxalaticus;

species of the genus Acinetobacter, such as Acinetobacter haylyi and Acinetobacter radioresistens;

species of the genus Alcaligenes, such as Alcaligenes faecalis;

species of the genus Delftia, such as Delftict acidovorans;

species of the genus Shimwellia, such as Shimwellia blattae;

species of the genus Serratia, such as Serratia grimesii, Serratia ficaria, Serratia fonticola, Serratia odorifera, Serratia plymuthica, Serratia entomophila, and Serratia nematodiphila.

Even a microorganism that originally has no abilities to catalyze the reactions A, C. andor F can also be used as the aforementioned host microorganism when an appropriate combination of nucleic acids that encode enzymes catalyzing the reactions A, C, and F is introduced into the microorganism to impart those production abilities.

As a microorganism that has an ability to produce adipic acid, a microorganism that has an ability to generate 3-oxoadipyl-CoA and coenzyme A from succinyl-CoA (the reaction A), an ability to generate 2,3-dehydroadipyl-CoA from 3-hydroxyadipyl-CoA through dehydration (the reaction C), an ability to reduce 2,3-dehydroadipyl-CoA to adipyl-CoA (the reaction D), and an ability to generate adipic acid from adipyl-CoA (the reaction G) is used. The microorganism with these production abilities can be used as a host microorganism to obtain a genetically modified microorganism with an ability to abundantly produce adipic acid.

Microorganisms that are speculated to originally have abilities to catalyze the above reactions A, C, D, and G include microorganisms of the genus Thermobifida, such as Thermobifida fusca.

Even a microorganism that originally has no abilities to catalyze the reactions A, C, D, and G can also be used as the aforementioned host microorganism when an appropriate combination of nucleic acids that encode enzymes catalyzing the reactions A, C, D, and G is introduced into the microorganism to impart those production abilities.

Specific examples of the enzymes that catalyze the reactions A and C to G are presented below.

As an enzyme that catalyzes the reaction A to generate 3-oxoadipyl-CoA, for example, an acyl transferase (P-ketothiolase) can be used. The acyl transferase is not limited to a particular number in the EC classification but is preferably an acyl transferase classified into EC 2.3.1.-, specifically including an enzyme classified as 3-oxoadipyl-CoA thiolase and classified into EC number 2.3.1.174, an enzyme classified as acetyl-CoA C-acetyltransferase and classified into EC number 2.3.1.9, and an enzyme classified as acetyl-CoA C-acyl transferase and classified into EC number 2.3.1.16. Among these, PaaJ from Escherichia coli strain MG1655 (NCBI-Protein ID: NP 415915), PcaF from Pseudomonas putida strain KT2440 (NCBI-Protein ID: NP 743536), and the like can be suitably used.

Whether or not the above acyl transferases can generate 3-oxoadipyl-CoA from succinyl-CoA and acetyl-CoA as substrates can be determined by measuring a decrease in NADH coupled with reduction of 3-oxoadipyl-CoA in a combination of the reaction catalyzed by purified acyl transferase to generate 3-oxoadipyl-CoA and a reaction catalyzed by purified 3-oxoadipyl-CoA reductase to reduce 3-oxoadipyl-CoA as a substrate. The specific measurement method is, for example, as follows.

Identification of acyl transferase activity: A PCR using the genomic DNA of a subject microorganism strain as a template is performed in accordance with routine procedures, to amplify a nucleic acid encoding an acyl transferase in the full-length form. The amplified fragment is inserted into the Suet site of pACYCDuet-1 (manufactured by Novagen), an expression vector for E. colt, in-frame with the histidine-tag sequence. The plasmid is introduced into E. coli BL21 (DE3), and expression of the enzyme is induced with isopropyl-P-thiogalactopyranoside (IPTG) in accordance with routine procedures and the enzyme is purified using the histidine tag from the culture fluid to obtain an acyl transferase solution. The acyl transferase activity can be determined by using the enzyme solution to prepare an enzymatic reaction solution with the following composition and measuring a decrease in absorbance at 340 ran coupled with oxidation of NADH at 30° C.

### (Enzymatic Reaction Solution)

100 mM Tris-HCl (pH 8.0)

10 mM MgCl₂

0.1 mM succinyl-CoA

0.2 mM acetyl-CoA

0.2 mM NADH

1 mM dithiothreitol

10 μgmL 3-oxoadipyl-CoA reductase

5 μgmL acyl transferase.

Whether or not an enzyme originally expressed in a host microorganism used in the present invention has acyl transferase activity can be determined by performing the above-described measurement using cell homogenate (cell free extract: CFE) instead of purified acyl transferase. The specific measurement method targeted to E. coli is, for example, as follows.

Preparation of CFE: A loopful of E. coli strain MG1655 to be subjected to the measurement of the activity is inoculated into 5 mL of a culture medium (culture medium composition: 10 gL tryptone, 5 gL yeast extract, 5 gL sodium chloride) adjusted to pH 7, and incubated at 30° C. with shaking for 18 hours. The obtained culture fluid is added to 5 mL of a culture medium (culture medium composition: 10 gL tryptone, 5 gL yeast extract, 5 gL sodium chloride, 2.5 mM ferulic acid, 2.5 mMp-coumaric acid, 2.5 mM benzoic acid, 2.5 mM cis,cis-muconic acid, 2.5 mM protocatechuic acid, 2.5 m1\4 catechol. 2.5 mM 30A, 2.5 mM 3-hydroxyadipic acid, 2.5 mM cc-hydromuconic acid. 2.5 mM adipic acid, 2.5 mM phenylethylamine) adjusted to pH 7. and incubated at 30° C. with shaking for 3 hours.

The obtained culture fluid is supplemented with 10 mL of 0.9% sodium chloride and then centrifuged to remove the supernatant from bacterial cells, and this operation is repeated three times in total to wash the bacterial cells. The washed bacterial cells are suspended in 1 mL of a Tris-HCl buffer composed of 100 mM Tris-HCl (pH 8.0) and 1 mM dithiothreitol, and glass beads (with a diameter of 0.1 mm) are added to the resulting suspension to disrupt the bacterial cells at 4° C. with an ultrasonic disruptor. The resulting bacterial homogenate is centrifuged to obtain the supernatant, and 0.5 mL of the supernatant is filtered through a UF membrane (Amicon Ultra-0.5mL 10K; manufactured by Merck Millipore) to remove the resulting filtrate, followed by application of 0.4 mL of the Tris-HCl buffer to the UF membrane, and this operation is repeated three times in total to remove low-molecular-weight impurities, and the resulting supernatant is then resuspended in the Tris-HCl buffer to a final volume of 0.1 mL, which is designated as CFE. Instead of purified enzyme, 0.05 mL of the CFE is added to a total of 0.1 mL of the enzymatic reaction solution to determine the enzymatic activity.

As an enzyme that catalyzes the reaction C to generate 2,3-dehydroadipyl-CoA, for example, an enoyl-CoA hydratase can be used. The enoyl-CoA hydratase is not limited by a particular number in the EC classification, and is preferably an enoyl-CoA hydratase classified into EC 4.2.1.-, specifically including an enzyme classified as enoyl-CoA hydratase or 2,3-dehydroadipyl-CoA hydratase and classified into EC 4.2.1.17. Among them, PaaF from Escherichia coli strain MG1655 (NCBI-ProteinlD: NP_415911), PaaF from Pseudomonas puhda strain KT2440 (NCBI-ProteinlD: NP_745427), and the like can be suitably used.

Since the reaction catalyzed by enoyl-CoA hydratase is generally reversible, whether or not an enoyl-CoA hydratase has an activity to catalyze a reaction that generates 2,3-dehydroadipyl-CoA from 3-hydroxyadipyl-CoA used as a substrate can be determined by detecting 3-hydroxyadipyl-CoA generated using purified enoyl-CoA hydratase with 2.3-dehydroadipyl-CoA used as a substrate thereof, which is prepared from α-hydromuconic acid through an enzymatic reaction. The specific measurement method is, for example, as follows.

The α-hydromuconic acid used in the above reaction can be prepared by a known method (for example, a method described in Reference Example 1 of WO 2016199858 A1).

Preparation of 2,3-dehydroadipyl-CoA solution: A PCR using the genomic DNA of Pseudomonas putida strain KT2440 as a template is performed in accordance with routine procedures, to amplify a nucleic acid encoding a CoA transferase (including peal and peal; NCBI-GenelDs: 1046613 and 1046612) in the full-length form. The amplified fragment is inserted into the Kpnl site of pRSF-⁻lb (manufactured by Novagen), an expression vector for E. coli, in-frame with the histidine-tag sequence. The plasmid is introduced into E. coli BL21 (DE3), and expression of the enzyme is induced with isopropyl-13-thiogalactopyranoside (IPTG) in accordance with routine procedures and the enzyme is purified using the histidine tag from the culture fluid to obtain a CoA transferase solution. The solution is used to prepare an enzymatic reaction solution for 2,3-dehydroadipyl-CoA preparation with the following composition, which is allowed to react at 30° C. for 10 minutes and then filtered through a UF membrane (Amicon Ultra-0.5mL 10K; manufactured by Merck Millipore) to remove the enzyme, and the obtained filtrate is designated as 2,3-dehydroadipyl-CoA solution.

### (Enzymatic Reaction Solution)

100 mM Tris-IICl (pH 8.0)

10 mM MgCI,

0.4 mM succinyl-CoA

2 mM ci-hydromuconic acid sodium salt

20 μgmL CoA transferase.

Identification of enoyl-CoA hydratase activity: A PCR using the genomic DNA of a subject microorganism strain as a template is performed in accordance with routine procedures, to amplify a nucleic acid encoding an enoyl-CoA hydratase in the lull-length form. The amplified fragment is inserted into the Ndel site of pET-16b (manufactured by Novagen), an expression vector for E. coil, in-frame with the histidine-tag sequence. The plasmid is introduced into E. coli BL21 (DE3), and expression of the enzyme is induced with isopropyl-β-thiogalactopyranoside (IPTG) in accordance with routine procedures and the enzyme is purified using the histidine tag from the culture fluid to obtain an enoyl-CoA hydratase solution. The solution is used to prepare an enzymatic reaction solution with the following composition, which is allowed to react at 30° C. for 10 minutes and then filtered through a UF membrane (Amicon Ultra-0.5mL 10K; manufactured by Merck Millipore) to remove the enzyme. The enoyl-CoA hydratase activity can be confirmed by detecting 3-hydroxyadipyl-CoA in the resulting filtrate on high-performance liquid chromatograph-tandem mass spectrometer (LC-MSMS) (Agilent Technologies, Inc.).

### (Enzymatic Reaction Solution)

100 mM Tris-HCl (pH 8.0)

10 mM MgCl,

300 μLmL 2,3-dehydroadipyl-CoA solution

1 mM dithiothreitol

20 ugmL enoyl-CoA hydratase.

Whether or not an enzyme originally expressed in a host microorganism used in the present invention has enoyl-CoA hydratase activity can be determined by adding 0.05 mL of the CFE, instead of purified enoyl-CoA hydratase, to a total of 0.1 mL of the enzymatic reaction solution and performing the above-described measurement. The specific CFE preparation method targeted to E. coli is as described for that used in determination of acyl transferase activity.

As an enzyme that catalyzes the reaction D to generate adipyl-CoA, for example, an enoyl-CoA reductase can be used. The enoyl-CoA reductase is not limited by a particular number in the EC classification, and is preferably an enoyl-CoA reductase classified into EC 1.3.-.-, specifically including an enzyme classified as trans-2-enoyl-CoA reductase and classified into EC 1.3.1.44, and an enzyme classified as acyl-CoA dehydrogenase and classified into EC 1.3.8.7. These specific examples are disclosed in, for example JP 2011-515111 A, J Appl Microbiol. 2015 Oct; 119 (4): 1057-63., and the like; among them, TER from Euglena gracilis strain Z (UniProtKB: Q5E⁻1590), Tfu 1647 from Thermobilida fitsca strain YX (NCBI-ProteinID: AAZ55682), DcaA from Acinetobacter baylyi strain ADPI (NCBI-ProteinID: AAL09094.1), and the like can be suitably used.

Whether or not an enoyl-CoA reductase has an activity to generate adipyl-CoA from 2,3-dehydroadipyl-CoA used as a substrate can be determined by measuring a decrease in NADH coupled with reduction of 2,3-dehydroadipyl-CoA in a reaction using purified enoyl-CoA reductase with 2,3-dehydroadipyl-CoA used as a substrate thereof, which is prepared from α-hydromuconic acid through another enzymatic reaction.

Preparation of α-hydromuconic acid and of 2,3-dehydroadipyl-CoA solution can be performed in the same manner as described above.

Identification of enoyl-CoA reductase activity: A PCR using the genomic DNA of a subject microorganism strain as a template is performed in accordance with routine procedures, to amplify a nucleic acid encoding an enoyl-CoA reductase in the full-length form. The amplified fragment is inserted into the NdeI site of pET-16b (manufactured by Novagen), an expression vector for E. coli, in-frame with the histidine-tag sequence. The plasmid is introduced into E. coli BL21 (DE3), and expression of the enzyme is induced with isopropyl-13-thiogalactopyranoside (IPTG) in accordance with routine procedures and the enzyme is purified using the histidine tag from the culture fluid to obtain an enoyl-CoA reductase solution. The enoyl-CoA reductase activity can be determined by using the enzyme solution to prepare an enzymatic reaction solution with the following composition and measuring a decrease in absorbance at 340 nm coupled with oxidation of NADH at 30° C.

### (Enzymatic Reaction Solution)

100 mM Tris-HCl (pH 8.0)

10 mM MgCl₂

300 μLmL 2,3-dehydroadipyl-CoA solution

0.2 mM NADH

1 mM dithiothreitol

20 μgmI. enoyl-CoA reductase.

Whether or not an enzyme originally expressed in a host microorganism used in the present invention has enoyl-CoA reductase activity can be determined by adding 0.05 mL of the CFE, instead of purified enoyl-CoA reductase, to a total of 0.1 mL of the enzymatic reaction solution and performing the above-described measurement. The specific CFE preparation method targeted to E. coli is as described for that used in determination of acyl transferase activity.

As an enzyme that catalyzes the reaction E to generate 3-hydroxyadipic acid, the reaction F to generate cc-hydromuconic acid, and the reaction G to generate adipic acid, for example, a CoA transferase or an acyl-CoA hydrolase, preferably a CoA transferase, can be used.

The CoA transferase is not limited by a particular number in the EC classification, and is preferably a CoA transferase classified into EC 2.8.3.-, specifically including an enzyme classified as CoA transferase or acyl-CoA transferase and classified into EC 2.8.3.6, and the like.

In the present invention, the term “CoA transferase” refers to an enzyme with activity (CoA transferase activity) to catalyze a reaction that generates carboxylic acid and succinyl-CoA from acyl-CoA and succinic acid used as substrates.

As an enzyme that catalyzes the reaction E to generate 3-hydroxyadipic acid and the reaction F to generate ct-hydromuconic acid, Peal and PcaJ from Pseudomonas putido strain KT2440 (NCBI-ProteinlDs: NP 746081 and NP 746082), and the like can be suitably used, among others.

As an enzyme that catalyzes the reaction G to generate adipic acid, Deal and DcaJ from Acinetobacter haylyi strain ADP1 (NCBI-ProteinlDs: CAG68538 and CAG68539), and the like can be suitably used.

Since the above enzymatic reactions are reversible, the CoA transferase activity against 3-hydroxyadipyl-CoA, 2,3-dehydroadipyl-CoA, or adipyl-CoA used as a substrate can be determined by detecting 3-hydroxyadipyl-CoA, 2,3-dehydroadipyl-CoA, or adipyl-CoA generated respectively using purified CoA transferase with 3-hydroxyadipic acid and succinyl-CoA, α-hydromuconic acid and succinyl-CoA, or adipic acid and succinyl-CoA used as substrates thereof. The specific measurement method is, for example, as follows.

Preparation of 3-hydroxyadipic acid: Preparation of 3-hydroxyadipic acid is performed according to the method described in Reference Example 1 of WO 2016199856 A1.

Identification of CoA transferase activity using 3-hydroxyadipic acid as a substrate: A PCR using the genomic DNA of a subject microorganism strain as a template is performed in accordance with routine procedures, to amplify a nucleic acid encoding a CoA transferase in the full-length form. The amplified fragment is inserted into the Kpnl site of pRSF-1 b (manufactured by Novagen), an expression vector for E. coli, in-frame with the histidine-tag sequence. The plasmid is introduced into E. coli BL21 (DE3), and expression of the enzyme is induced with isopropyl-1-thiogalactopyranoside (IPTG) in accordance with routine procedures and the enzyme is purified using the histidine tag from the culture fluid to obtain a CoA transferase solution. The solution is used to prepare an enzymatic reaction solution with the following composition, which is allowed to react at 30° C. for 10 minutes and then filtered through a UF membrane (Amicon Ultra-0.5mL 10K; manufactured by Merck Millipore) to remove the enzyme. The CoA transferase activity can be confirmed by detecting 3-hydroxyadipyl-CoA in the resulting filtrate on high-performance liquid chromatograph-tandem mass spectrometer (LC-MSMS) (Agilent Technologies, Inc.).

### (Enzymatic Reaction Solution)

100 mM Tris-HCl (pII 8.0)

10 mM MgCl,

0.4 mM succinyl-CoA

2 mM 3-hydroxyadipic acid sodium salt

20 μgmL CoA transferase.

Preparation of α-hydromuconic acid: Preparation of α-hydromuconic acid is performed according to the method described in Reference Example 1 of WO 2016199858 A1.

Identification of CoA transferase activity using α-hydromuconic acid as a substrate: A PCR using the genomic DNA of a subject microorganism strain as a template is performed in accordance with routine procedures, to amplify a nucleic acid encoding a CoA transferase in the full-length lot III. The amplified fragment is inserted into the Kpnl site of pRSF-lb (manufactured by Novagen), an expression vector for E. coli, in-frame with the histidine-tag sequence. The plasmid is introduced into E. coli BL21 (DE3), and expression of the enzyme is induced with isopropyl-β-thiogalactopyranoside (IPTG) in accordance with routine procedures and the enzyme is purified using the histidine tag from the culture fluid to obtain a CoA transferase solution. The solution is used to prepare an enzymatic reaction solution with the following composition, which is allowed to react at 30° C. for 10 minutes and then filtered through a UF membrane (Amicon Ultra-0.5mL 10K; manufactured by Merck Millipore) to remove the enzyme. The CoA transferase activity can be confirmed by detecting 2,3-dehydroadipyl-CoA in the resulting filtrate on high-performance liquid chromatograph-tandem mass spectrometer (LC-MSMS) (Agilent Technologies, Inc.).

### (Enzymatic Reaction Solution)

100 mM Tris-HCl (pH 8.0)

10 niM MgCl₂

0.4 mM succinyl-CoA

2 mM α-hydromuconic acid sodium salt

20 μgmL CoA transferase.

Identification of CoA transferase activity using adipic acid as a substrate: A PCR using the genomic DNA of a subject microorganism strain as a template is performed in accordance with routine procedures, to amplify a nucleic acid encoding a CoA transferase in the full-length form. The amplified fragment is inserted into the Kpnl site of pRSF-1 b (manufactured by Novagen), an expression vector for E. coli, in-frame with the histidine-tag sequence. The plasmid is introduced into E. coli BL21 (DE3), and expression of the enzyme is induced with isopropyl-β-thiogalactopyranoside (IPTG) in accordance with routine procedures and the enzyme is purified using the histidine tag from the culture fluid to obtain a CoA transferase solution. The solution is used to prepare an enzymatic reaction solution with the following composition, which is allowed to react at 30° C. for 10 minutes and then filtered through a UF membrane (Amicon Ultra-0.5mL 10K; manufactured by Merck Millipore) to remove the enzyme. The CoA transferase activity can be confirmed by detecting adipyl-CoA in the resulting filtrate on high-performance liquid chromatograph-tandem mass spectrometer (LC-MSMS) (Agilent Technologies, Inc.).

### (Enzymatic Reaction Solution)

100 mM Tris-HCl (pH 8.0)

10 mM MgCl₂

0.4 mM succinyl-CoA

2 mM adipic acid sodium salt

20 μgmL CoA-transferase.

Whether or not an enzyme originally expressed in a host microorganism used in the present invention has CoA transferase activity can be determined by adding 0.05 mL of the CFE, instead of purified CoA transferase, to a total of 0.1 mL of the enzymatic reaction solution and performing the above-described measurement. The specific CFE preparation method targeted to E. coli is as described for that used in determination of acyl transferase activity.

Either the polypeptides described in (a) to (c) or the 3-hydroxybutyryl-CoA dehydrogenase in the present invention is characterized by having higher activity than 3-oxoadipyl-CoA reductases used in conventional techniques. In this respect, the phrase “higher activity” refers to production of 3-hydroxyadipic acid, α-hydromuconic acid, or adipic acid with a higher yield in a genetically modified microorganism expressing any one of the polypeptides than in a genetically modified microorganism expressing a conventional 3-oxoadipyl-CoA reductase when those microorganisms are derived from the same host microorganism species and are cultured under the same expression conditions in a culture medium containing a carbon source as a material for fermentation. In this respect, the yield of 3-hydroxyadipic acid is calculated according to the formula (2). The yield of u-hydromuconic acid or adipic acid is calculated according to the formula (2), where 3-hydroxyadipic acid is replaced by u-hydromuconic acid or adipic acid, respectively.

$$Formula ( 2 )$$

$$Yield ( \% ) = amount of generated 3 \text{-} hydroxyadipic acid ( mol ) \text{/} amount of consumed carbon source ( mol ) \times 100$$

The specific method to confirm the higher activity of either the polypeptides described in (a) to (c) or the 3-hydroxybutyryl-CoA dehydrogenase in the present invention compared to the activity of 3-oxoadipyl-CoA reductases used in conventional techniques is as follows. The pBBR1 MCS-2 vector, which is able to self-replicate in E. coli (ME Kovach, (1995), Gene 166: 175-176), is cleaved with Xhol to obtain pBBR1MCS-2Xhol. To integrate a constitutive expression promoter into the vector, an upstream 200-b region (SEQ ID NO: 186) of gapA (NCBI Gene ID: NC 000913.3) is amplified by PCR using the genomic DNA of Escherichia coli K-12 MG1655 as a template in accordance with routine procedures (for example, primers represented by SEQ ID NOs: 187 and 188 are used), and the obtained fragment and the pBBR1MCS-2Xhol are ligated together using the In-Fusion HD Cloning Kit (manufactured by Takara Bio Inc.) to obtain the plasmid pBBR1MCS-2::Pgap. The pBBR1MCS-2::Pgap is cleaved with Scol to obtain pBBR1MCS-2::PgapScaI. A nucleic acid encoding an acyl transferase in the full length form is amplified by PCR in accordance with routine procedures (for example, primers represented by SEQ ID NOs: 190 and 191 are used), and the obtained fragment and pBBR1MCS-2::PgapScaI are ligated together using the In-Fusion HD Cloning Kit to obtain the plasmid pBBR1MCS-2::AT. The pBBR⁻1MCS-2::AT is cleaved with Hpal to obtain pBBR1MCS-2::ATIIpal. A nucleic acid encoding a CoA transferase in the full length form is amplified by PCR in accordance with routine procedures (for example, primers represented by SEQ ID NOs: 194 and 195 are used), and the obtained fragment and pBBR1MCS-2::ATHpaI are ligated together using “the In-Fusion HD Cloning Kit” to obtain the plasmid pBBR1MCS-2::ATCT.

On the other hand, the pACYCDuet-1 expression vector (manufactured by Novagen), which is able to self-replicate in E. coli, is cleaved with BaivHl to obtain pACYCDuet-1BamHI. A nucleic acid encoding a polypeptide represented by any one of SEQ ID NOs: 1 to 86 or encoding a conventionally used 3-oxoadipyl-CoA reductase, is amplified by PCR in accordance with routine procedures (for example, primers represented by SEQ ID NOs: 196 and 197 are used), and the obtained fragment and pACYCDuet-1BamIII are ligated together using the In-Fusion HD Cloning Kit (manufactured by Takara Bio Inc.) to obtain a plasmid that expresses the polypeptide represented by any one of SEQ ID NOs: 1 to 86 or expresses the conventionally used 3-oxoadipyl-CoA reductase.

The obtained plasmid and the pBBR1MCS-2::ATCT are introduced into E. coli strain BL21 (DE3) by electroporation (NM Calvin. PC Hanawalt. J. Bacteriol, 170 (1988), pp. 2796-2801). A loopful of the strain after the introduction is inoculated into 5 mL of the culture medium I (10 gL Bacto Tryptone (manufactured by Difco Laboratories), 5 gL Bacto Yeast Extract (manufactured by Difco Laboratories), 5 gL sodium chloride, 25 ugmL kanamycin, and 15 μgmL chloramphenicol) adjusted to pH 7, and incubated at 30° C. with shaking at 120 min⁻¹ :for 18 hours. Subsequently, 0.25 mL of the culture fluid is added to 5 mL of the culture medium II (10 gL succinic acid, 10 gL glucose, 1 gl, ammonium sulfate, 50 mM potassium phosphate,0.025 gL magnesium sulfate,0.0625 mgL iron sulfate, 2.7 mgL manganese sulfate, 0.33 mgt, calcium chloride, 1.25 gL sodium chloride, 2.5 gL Bacto Tryptone. 1.25 gL Bacto Yeast Extract. 25 μgmL kanamycin, 15 μgmL chloramphenicol, and 0.01 mM IPTG) adjusted to plI 6.5. and incubated at 30° C. with shaking at 120 min^{-I} for 24 hours. The supernatant separated from bacterial cells by centrifugation of the culture fluid is processed by membrane treatment using Millex-GV (0.22 μm; PVDF; manufactured by Merck KGaA), and the resulting filtrate is analyzed to measure the 3-hydroxyadipic acid and carbon source concentrations in the culture supernatant. Quantitative analysis of 3-hydroxyadipic acid on LC-MSMS is performed under the following conditions.
HPLC: 1290 Infinity (manufactured by Agilent Technologies, Inc.)
Column: Synergi hydro-RP (manufactured by Phenomenex Inc.), length: 100 mm,
internal diameter: 3 mm, particle size: 2.5 um

Mobile phase: 0.1% aqueous formic acid solution methanol =7030
Flow rate: 0.3 mLmin
Column temperature: 40° C.
LC detector: DAD (210 nm)
MSMS: Triple-Quad LCMS (manufactured by Agilent Technologies, Inc.)
Ionization method: ESI in negative mode.

Quantitative analysis of carbon sources, such as sugars and succinic acid, on HPLC is performed under the following conditions.
HPLC: Shimazu Prominence (manufactured by Shimadzu Corporation)
Column: Shodex Sugar SH1011 (manufactured by Showa Denko K.K.), length: 300 mm, internal diameter: 8 mm, particle size: 6 μm
Mobile phase: 0.05M aqueous sulfuric acid solution
Flow rate: 0.6 mLmin
Column temperature: 65° C.
Detector: RI.

When a nucleic acid encoding any one selected from the group of the acyl transferase, the CoA transferase, the enoyl-CoA hydratase, and the enoyl-CoA reductase is introduced into a host microorganism in the present invention, the nucleic acid may be artificially synthesized based on the amino acid sequence information of the enzyme in a database, or isolated from the natural environment. In cases where the nucleic acid is artificially synthesized, the usage frequency of codons corresponding to each amino acid in the nucleic acid sequence may be changed depending on the host microorganism into which the nucleic acid is introduced.

In the present invention, the method of introducing a nucleic acid encoding any one selected from the group of the acyl transferase, the CoA transferase, the enoyl-CoA hydratase, and the enoyl-CoA reductase into the host microorganism method is not limited to a particular method; for example, a method in which the nucleic acid is integrated into an expression vector capable of autonomous replication in the host microorganism and then introduced into the host microorganism, a method in which the nucleic acid is integrated into the genome of the host microorganism, and the like can he used.

In cases where a nucleic acid encoding any one of the enzymes is isolated from the natural environment, the sources of the genes are not limited to particular organisms, and examples of the organisms include those of the genus Acinetobacter, such as Acinetobacter baylyi and Acinetobacter radioresistens; the genus Aerobacter, such as Aerobacter cloacae; the genus Alcaligenes, such as Alcaligenesfaecalis; the genus Bacillus, such as Bacillus badius, Bacillus magaterium, and Bacillus roseus; the genus Brevibacterium. such as Brevibacterium todinum; the genus Corynebacterium, such as Corynebacterium acetoacidophilum, Corynebacterium acetoglutamicum, Corynebacterium ammoniagenes, and Corynebacterium glutamicum; the genus Cupriavidus, such as Cupriavidus metallidurans, Cupriavidus necator, Cupriavidus numazuensis, and Cupriavidus oxalaticus; the genus Delflia, such as Delftia acidovorans; the genus Escherichia, such as Escherichia coli and Escherichia lergusonii; the genus Hafnia, such as Hafnia alvei; the genus Microbacterium, such as Microbacterium ammoniaphilum; the genus Nocardioides, such as Nocardioides alhus; the genus Planomicrohium, such as Planomicrobium okeanokoites; the genus Pseudomonas, such as Pseudomonas azotoformans, Pseudomonas chlororaphis, Pseudomonas fluorescens, Pseudomonas Pseudomonas putida, and Pseudomonas reptilivora; the genus Rhizobium, such as Rhizobium radiohacter; the genus Rhodosporidium, such as Rhodosporidium toruloides; the genus Saccharomyces, such as Saccharomyces cerevisiae; the genus Serratia, such as Serratia entoinophila, Serratialicaria, Serratia fonticola, Serratia grimesii, Serratia nematodiphila, Serratia odorilera, and Serratia plymuthica; the genus Shimwellia, such as Shimwellia blattae; the genus Streptomyces, such as Streptomyces vinaceus, Streptomyces karnatakensis, Streptomyces olivaceus, and Streptomyces vinaceus; the genus Yarrowia, such as Yarrowia lipolytica; the genus Yersinia, such as Yersinia ruckeri; the genus Euglena, such as Euglena gracilis; and the genus Thermobifida, such as Thermobifidalitsca. Preferably, the organisms are those of the genera Acinetobacter, Corynebacterium, Escherichia, Pseudomonas, Serratia, Euglena, and Thermobifida.

When a nucleic acid encoding a polypeptide expressed in the present invention is integrated into an expression vector or the genome of a host microorganism, the nucleic acid being integrated into the expression vector or the genome is preferably composed of a promoter, a ribosome-binding sequence, a nucleic acid encoding the polypeptide to be expressed, and a transcription termination sequence, and may additionally contain a gene that controls the activity of the promoter.

The promoter used in the present invention is not limited to a particular promoter, provided that the promoter drives expression of the enzyme in the host microorganism; examples of the promoter include gap promoter, trp promoter, lac promoter, toe promoter, and T7 promoter.

In cases where an expression vector is used in the present invention to introduce the nucleic acid or to enhance the expression of the polypeptide, the expression vector is not limited to a particular vector, provided that the vector is capable of autonomous replication in the microorganism; examples of the vector include pBBR1MCS vector, pBR322 vector, pMW vector, pET vector, pRSF vector, pCDF vector, pACYC vector, and derivatives of the above vectors.

In cases where a nucleic acid for genome integration is used in the present invention to introduce the nucleic acid or to enhance the expression of the polypeptide, the nucleic acid for genome integration is introduced by site-specific homologous recombination. The method for site-specific homologous recombination is not limited to a particular method, and examples of the method include a method in which λ Red recombinase and FLP recombinase are used (Proc Natl Acad Sci U.S.A. 2000 Jun. 6; 97 (12): 6640-6645.), and a method in which X Red recombinase and the soeB gene are used (Biosci Biotechnol Biochem. 2007 December; 71 (12):2905-11.).

The method of introducing the expression vector or the nucleic acid for genome integration is not limited to a particular method, provided that the method is for introduction of a nucleic acid into a microorganism; examples of the method include the calcium ion method (Journal of Molecular Biology, 53, 159 (1970)), and electroporation (N M Calvin, P C Hanawalt. J. Bacteriol, 170 (1988), pp. 2796-2801).

In the present invention, a genetically modified microorganism in which a nucleic acid encoding a 3-oxoadipyl-CoA reductase is introduced or expression of the corresponding polypeptide is enhanced is cultured in a culture medium, preferably a liquid culture medium, containing a carbon source as a material for fermentation which can be used by ordinary microorganisms. The culture medium used contains, in addition to the carbon source that can be used by the genetically modified microorganism, appropriate amounts of a nitrogen source, inorganic salts, and, if necessary, organic trace nutrients such as amino acids and vitamins. Any of natural and synthetic culture media can be used as long as the medium contains the above-described nutrients.

The material for fermentation is a material that can be metabolized by the genetically modified microorganism. The term “metabolize” refers to conversion of a chemical substance, which a microorganism has taken up from the extracellular environment or intracellularly generated from a different chemical substance, to another chemical substance through an enzymatic reaction. Sugars can be suitably used as the carbon source. Specific examples of the sugars include monosaccharides, such as glucose, sucrose, fructose, galactose, mannose, xylose, and arabinose; disaccharides and polysaccharides formed by linking these monosaccharides; and saccharified starch solution, molasses, and saccharified solution from cellulose-containing biomass, each containing any of those saccharides.

Other than the above sugars, succinic acid, a substrate of the CoA transferase, can also be added to the culture medium for efficient production of 3-hydroxyadipic acid, α-hydromuconic acid, andor adipic acid.

The above-listed carbon sources may be used individually or in combination. When a carbon source is added, the concentration of the carbon source in the culture medium is not particularly limited, and can be appropriately selected depending on the type of the carbon source; in the case of sugars, the concentration is preferably from 5 gL to 300 gL; in the case of succinic acid, the concentration is preferably from 0.1 gL to 100 gL.

As the nitrogen source used for culturing the genetically modified microorganism, for example, ammonia gas, aqueous ammonia, ammonium salts, urea, nitric acid salts, other supportively used organic nitrogen sources, such as oil cakes, soybean hydrolysate, casein degradation products, other amino acids; vitamins, corn steep liquor, yeast or yeast extract, meat extract, peptides such as peptone, and bacterial cells and hydrolysate of various fermentative bacteria can be used. The concentration of the nitrogen source in the culture medium is not particularly limited, and is preferably from 0.1 gL to 50 gL.

As the inorganic salts used for culturing the genetically modified microorganism, for example, phosphoric acid salts, magnesium salts, calcium salts, iron salts, and manganese salts can be appropriately added to the culture medium and used.

The culture conditions for the genetically modified microorganism to produce 3-hydroxyadipic acid, α-hydromuconic acid, andor adipic acid are set by appropriately adjusting or selecting, for example, the culture medium with the above composition, culture temperature, stirring speed, pH, aeration rate, and inoculation amount, depending on, for example, the species of the genetically modified microorganism and external conditions.

The pH range of the culture is not specifically limited, provided that the genetically modified microorganism can be grown in the pH range. However, the pH range is preferably from pH 5 to 8, more preferably from pH 5.5 to 6.8.

Although the range of aeration rates in the culture is not specifically limited, as long as 3-hydroxyadipic acid, α-hydromuconic acid, andor adipic acid can be produced under the aeration conditions. It is desired that oxygen remain in the gaseous phase andor liquid phase in a culture container for good growth of the mutant microorganism at least at the start of incubation.

In cases where foam is formed in a liquid culture, an antifoaming agent such as a mineral oil, silicone oil, or surfactant may be appropriately added to the culture medium.

After a recoverable amount of 3-hydroxyadipic acid, ct-hydromuconic acid, andor adipic acid is produced during culturing of the microorganism, the produced products can be recovered. The produced products can be recovered, for example isolated, according to a commonly used method, in which the culturing is stopped once a product of interest is accumulated to an appropriate level, and the fermentation product is collected from the culture. Specifically, the products can be isolated from the culture by separation of bacterial cells through, for example, centrifugation or filtration prior to, for example, column chromatography, ion exchange chromatography, activated charcoal treatment, crystallization, membrane separation, or distillation. More specifically, examples include, but are not limited to, a method in which an acidic component is added to salts of the products, and the resulting precipitate is collected; a method in which water is removed from the culture by concentration using, for example, a reverse osmosis membrane or an evaporator to increase the concentrations of the products and the products andor salts of the products are then crystallized and precipitated by cooling or adiabatic crystallization to recover the crystals of the products andor salts of the products by, for example, centrifugation or filtration; and a method in which an alcohol is added to the culture to produce esters of the products and the resulting esters of the products are subsequently collected by distillation and then hydrolyzed to recover the products. These recovery methods can be appropriately selected and optimized depending on, for example, physical properties of the products.

### EXAMPLES

The present invention will be specifically described below with reference to examples.

### Reference Example 1

Production of plasmids each expressing an enzyme catalyzing a reaction to generate 3-oxoadipyl-CoA and coenzyme A (the reaction A), an enzyme catalyzing a reaction to generate 3-hydroxyadipic acid from 3-hydroxyadipyl-CoA (the reaction E) and a reaction to generate α-hydromuconic acid from 2,3-dehydroadipyl-CoA (the reaction F), and a polypeptide represented by SEQ ID NO: 1, 2, 3, 4, 5, 6, or 7

The pBBR1MCS-2 vector, which is capable of autonomous replication in E. coil (ME Kovach, (1995). Gene 166: 175-176), was cleaved with Xhol to obtain pBBR1MCS-2Xhol. To integrate a constitutive expression promoter into the vector, primers (SEQ ID NOs: 187 and 188) were designed to amplify the upstream 200-b region (SEQ ID NO: 186) of gapzI (NCBI Gene ID: NC 000913.3) by PCR using the genomic DNA of Escherichia coil K-12 MG1655 as a template, and a PCR reaction was performed in accordance with routine procedures. The obtained fragment and pF3BR1MCS-2XhoI were ligated together using the In-Fusion HD Cloning Kit (manufactured by Takara Bio Inc.), and the resulting plasmid was introduced into E. coli strain DH5a. The nucleotide sequence on the plasmid isolated from the obtained recombinant E. coli strain was confirmed in accordance with routine procedures, and the plasmid was designated as pBBRIMCS-2::Pgap. Then, the pBBR1MCS-2::Pgap was cleaved with Seal to obtain pBBR1MCS-2::PgapScaI. For amplification of a gene encoding an enzyme catalyzing the reaction A, primers (SEQ ID NOs: 190 and 191) were designed to amplify the full length of the acyl transferase gene pcaF (NCBI Gene ID: 1041755, SEQ ID NO: 189) by PCR using the genomic DNA of Pseudomonas putida strain KT2440 as a template, and a PCR reaction was performed in accordance with routine procedures. The obtained fragment and the pBBR1MCS-2::PgapScaI were ligated together using the In-Fusion HD Cloning Kit, and the resulting plasmid was introduced into E. coli strain DH5a. The nucleotide sequence on the plasmid isolated from the obtained recombinant strain was confirmed in accordance with routine procedures, and the plasmid was designated as pBBR1MCS-2::AT. Then, the pBBR1MCS-2::AT was cleaved with Hpal to obtain pBBR1MCS-2::ATHpaI. For amplification of a gene encoding an enzyme catalyzing the reactions D and F, primers (SEQ ID NOs: 194 and 195) were designed to amplify a continuous sequence including the full lengths of genes together encoding a CoA transferase, peal and peal (NCBI Gene IDs: 1046613 and 1046612, SEQ ID NOs: 192 and 193) by PCR using the genomic DNA of Pseudomonas putida strain KT2440 as a template, and a PCR reaction was performed in accordance with routine procedures. The obtained fragment and the pBBR1MCS-2::ATHpal were ligated together using the In-Fusion HD Cloning Kit, and the resulting plasmid was introduced into E. coli strain DH5a. The nucleotide sequence on the plasmid isolated from the obtained recombinant strain was confirmed in accordance with routine procedures, and the plasmid was designated as pBBR1MCS-2::ATCT.

The pBBR1MCS-2::ATCT was cleaved with Seal to obtain pBBR1MCS-2::ATCTSca1. For amplification of a nucleic acid encoding a polypeptide represented by SEQ ID NO: 1, primers (SEQ ID NOs: 196 and 197) were designed to amplify the nucleic acid represented by SEQ ID NO: 87 through PCR using the genomic DNA of Serratia marcescens strain ATCC13880 as a template, and a PCR reaction was performed in accordance with routine procedures. For amplification of a nucleic acid encoding a polypeptide represented by SEQ ID NO: 2, primers (SEQ ID NOs: 198 and 199) were designed to amplify the nucleic acid represented by SEQ ID NO: 88 through PCR using the genomic DNA of Serratia nematodiphila strain DSM21420 as a template, and a PCR reaction was performed in accordance with routine procedures. For amplification of a nucleic acid encoding a polypeptide represented by SEQ ID NO: 3, primers (SEQ ID NOs: 200 and 201) were designed to amplify the nucleic acid represented by SEQ ID NO: 89 through PCR using the genomic DNA of Serratia plymuthica strain NBRC102599 as a template, and a PCR reaction was performed in accordance with routine procedures. For amplification of a nucleic acid encoding a polypeptide represented by SEQ ID NO: 4, primers (SEQ ID NOs: 202 and 203) were designed to amplify the nucleic acid represented by SEQ ID NO: 90 through PCR using the genomic DNA of Serratia proteamaculans strain 568 as a template, and a PCR reaction was performed in accordance with routine procedures. For amplification of a nucleic acid encoding a polypeptide represented by SEQ ID NO: 5, primers (SEQ ID NOs: 204 and 205) were designed to amplify the nucleic acid represented by SEQ ID NO: 91 through PCR using the genomic DNA of Serratia ureilytica strain Lr54 as a template, and a PCR reaction was performed in accordance with routine procedures. For amplification of a nucleic acid encoding a polypeptide represented by SEQ ID NO: 6, primers (SEQ ID NOs: 206 and 207) were designed to amplify the nucleic acid represented by SEQ ID NO: 92 through PCR using the genomic DNA of Serratia sp. strain BW106 as a template, and a PCR reaction was performed in accordance with routine procedures. For amplification of a nucleic acid encoding a polypeptide represented by SEQ ID NO: 7, primers (SEQ ID NOs: 208 and 209) were designed to amplify the nucleic acid represented by SEQ ID NO: 93 through PCR using the genomic DNA of Serratia liquefaciens strain FK01 as a template, and a PCR reaction was performed in accordance with routine procedures. Each of the obtained fragments and the pBBR1MCS-2::ATCTScaI were ligated together using the In-Fusion HD Cloning Kit (manufactured by Takara Bio Inc.), and each of the resulting plasmids was introduced into E. coli strain DH5a. The nucleotide sequence on the plasmid isolated from each of the obtained recombinant strains was confirmed in accordance with routine procedures.

The plasmid for expression of the polypeptide represented by SEQ ID NO: 1 was designated as “pBBR1MCS-2::ATCTOR1”; the plasmid for expression of the polypeptide represented by SEQ ID NO: 2 was designated as “pBBR1MCS-2::ATCTOR2”; the plasmid for expression of the polypeptide represented by SEQ ID NO: 3 was designated as “pBFIR1MCS-2::ATCTOR3”; the plasmid for expression of the polypeptide represented by SEQ ID NO: 4 was designated as “pBBR1N1CS-2::ATCTOR4”; the plasmid for expression of the polypeptide represented by SEQ ID NO: 5 was designated as “pBBR1MCS-2::ATCTOR5”; the plasmid for expression of the polypeptide represented by SEQ ID NO: 6 was designated as “pBBR1MCS-2::ATCTOR6”; and the plasmid for expression of the polypeptide represented by SEQ ID NO: 7 was designated as “pBBR1MCS-2::ATCTOR7”; and these plasmids are listed in Table 6.

TABLE 6

| | | | SEQ |
|---|---|---|---|
| | | | ID |
| Plasmid | Originating organism | Gene ID | NO: |
| pBBR1MCS- | Serratia marcescens ATCC | JMPQ01000047.1 | 87 |
| 2::ATCTOR1 | 13880 | | |
| pBBR1MCS- | Serratia nematodiphila | JPUX00000000.1 | 88 |
| 2::ATCTOR2 | DSM21420 | | |
| pBBR1MCS- | Serratia plymuthica | BCTU01000013.1 | 89 |
| 2::ATCTOR3 | NBRC102599 | | |
| pBBR1MCS- | Serratia proteamaculans | CP000826.1 | 90 |
| 2::ATCTOR4 | 568 | | |
| pBBR1MCS- | Serratia ureilytica Lr5/4 | JSFB01000001 | 91 |
| 2::ATCTOR5 | | | |
| pBBR1MCS- | Serratia sp. BW106 | MCGS01000002.1 | 92 |
| 2::ATCTOR6 | | | |
| pBBR1MCS- | Serratia liquefaciens FK01 | CP006252.1 | 93 |
| 2::ATCTOR7 | | | |

### Reference Example 2

Production of a plasmid for expression of an enzyme catalyzing a reaction to generate 2,3-dehydroadipyl-CoA from 3-hydroxyadipyl-CoA (the reaction C)

The pMW119 expression vector (manufactured by Nippon Gene Co., Ltd.), which is capable of autonomous replication in E. coli, was cleaved with Sad to obtain pMW119SacI. To integrate a constitutive expression promoter into the vector, primers (SEQ ID NOs: 210 and 211) were designed to amplify the upstream 200-b region (SEQ ID NO: 186) of gapA (NCBI Gene ID: NC 000913.3) by PCR using the genomic DNA of Escherichia coil K-12 MG1655 as a template, and a PCR reaction was performed in accordance with routine procedures. The obtained fragment and the pMW119SacI were ligated together using the In-Fusion HD Cloning Kit (manufactured by Takara Bio Inc.), and the resulting plasmid was introduced into E. coli strain DH5ct. The nucleotide sequence on the plasmid isolated from the obtained recombinant E. coli strain was confirmed in accordance with routine procedures, and the plasmid was designated as pMW1 19::Pgap. Then, the pMW1 19::Pgap was cleaved with Sphl to obtain pMW119::PgapSphl. For amplification of a gene encoding an enzyme catalyzing the reaction C, primers (SEQ ID NOs: 212 and 213) were designed to amplify the full length of the enoyl-CoA hydratase gene paaF (NCBI Gene ID: 1046932, SEQ ID NO: 176) by PCR using the genomic DNA of Pseudomonas puticia strain KT2440 as a template, and a PCR reaction was performed in accordance with routine procedures. The obtained fragment and the pMW119::PgapSphl were ligated together using the In-Fusion HD Cloning Kit (manufactured by Takara Bio Inc.), and the resulting plasmid was introduced into E. coli strain DH5a. The nucleotide sequence on the plasmid isolated from the obtained recombinant strain was confirmed in accordance with routine procedures. The obtained plasmid was designated as “pMW119::EH”.

### Reference Example 3

Production of plasmids each expressing an enzyme catalyzing a reaction to generate 3-oxoadipyl-CoA and coenzyme A from acetyl-CoA and succinyl-CoA (the reaction A), an enzyme catalyzing a reaction to generate adipic acid from adipyl-CoA (the reaction G), and a polypeptide represented by SEQ ID NO: 1, 2, 3, 4, 5, 6, or 7

For amplification of a gene encoding an enzyme catalyzing the reaction G, primers (SEQ ID NOs: 216 and 217) were designed to amplify a continuous sequence including the full lengths of genes together encoding a CoA transferase, dcaI and dcaf (NCBI Gene ID: CR543861.1, SEQ ID NOs: 214 and 215) by PCR using the genomic DNA of Acinetobacter baylyi strain ADPI as a template, and a PCR reaction was performed in accordance with routine procedures. The obtained fragment and each of the fragments obtained by cleaving the pBBR1MCS-2::ATCTORE pBBR1MCS-2::ATCTOR2, pBBR1MCS-2::ATCTOR3, pBBR1MCS-2::ATCTOR4, pBBR1MCS-2::ATCTOR5, pBBR1MCS-2::ATCTOR6, and pBBR1MCS-2::ATCTOR7 with Hpal, which were produced in Reference Example 1, were ligated together using the In-Fusion HD Cloning Kit, and each of the resulting plasmids was introduced into E. coli strain DH5u. The nucleotide sequences on the plasmids isolated from the obtained recombinant strains were confirmed in accordance with routine procedures, and the plasmids were designated as pBBR1MCS-2::ATCT2OR1, pBBIUMCS-2::ATCT2OR2, pBBR1MCS-2::ATCT2OR3, pBBR1MCS-2::ATCT2OR4, pBBR1MCS-2::ATCT2OR5, pBBR1MCS-2::ATCT2OR6, and pBBR1MCS-2::ATCT2OR7.

### Reference Example 4

Production of a plasmid for expression of enzymes catalyzing a reaction to generate 2,3-dehydroadipyl-CoA from 3-hydroxyadipyl-CoA (the reaction C) and a reaction to generate adipyl-CoA from 2,3-dehydroadipyl-CoA (the reaction D)

The pMW119::EH was cleaved with HindIll to obtain pMW119::EHHindIII. For amplification of a gene encoding an enzyme catalyzing the reaction D, primers (SEQ ID NOs: 219 and 220) were designed to amplify the full length of dcaA (NCBI-Protein ID: AAL09094.1, SEQ ID NO: 218) from Acinetobacter baylyi strain ADP1 by PCR, and a PCR reaction was performed in accordance with routine procedures. The obtained fragment and the pMW119::EHHindIII were ligated together using the In-Fusion HD Cloning Kit (manufactured by Takara Bio Inc.), and the resulting plasmid was introduced into E. coli strain DII5u. The nucleotide sequence on the plasmid isolated from the obtained recombinant strain was confirmed in accordance with routine procedures, and the plasmid was designated as pMW119::EHER.

### Example 1

Generation of a Mutant Microorganism of the Genus Serratia with Impaired Pyruvate Kinase Function

Genes encoding the pyruvate kinase of a microorganism of the genus Serratia, pykF and pykA, were disrupted to generate a mutant microorganism of the genus Serratia with impaired pyruvate kinase function.

The procedure for disrupting pykF and pykA followed the method described in Proc Natl Acad Sci U S A., 2000 Jun. 6, 97(12): 6640-6645.

Generation of a Mutant Microorganism Of The Genus Serratia Deficient in pykF

A PCR reaction was performed using pKD4 as a template and oligo DNAs represented by SEQ ID NOs: 221 and 222 as primers to obtain a PCR fragment of 1.6 kb in length for disruption of pykF. A FRT recombinase expression plasmid, pKD46, was introduced into Serratia grime,sli strain NBRC13537, and an ampicillin-resistant strain was obtained. The obtained strain was inoculated into 5 mL of LB medium containing 500 ugmL ampicillin and was cultured at 30° C. with shaking for 1 day. Subsequently, 0.5 mL of the culture fluid was inoculated into 50 mL of LB medium containing 500 ugmI, ampicillin and 50 mM arabinose and was cultured in rotation at 30° C. for 2 hours. The culture fluid was cooled on ice for 20 minutes, and the bacterial cells were then washed with 10% (ww) glycerol three times. The washed pellet was suspended in 100 ,AL of 10% (ww) glycerol and mixed with 5 μL of the PCR fragment, and the mixture was then cooled in an electroporation cuvette on ice for 10 minutes. Electroporation was performed using a Gene Pulser electroporator (manufactured by Bio-Rad Laboratories, Inc.; 3 kV, 200 D, 25 μF), and 1 mL of SOC medium was added to the electroporation cuvette immediately after the electroporation, and the bacterial cells in the cuvette were incubated at 30° C. with shaking for 2 hours. The total volume of the culture was applied to LB agar medium containing 25 ugmL kanamycin and was incubated at 30° C. for 1 day. Direct colony PCR was performed on the resulting kanamycin-resistant strains to confirm the deletion of the gene of interest and the insertion of a kanamycin resistance gene from the length of the amplified band. Oligo DNA primers represented by SEQ ID NOs: 223 and 225 were used.

Subsequently, one of the kanamycin-resistant strains was inoculated into 5 mL of LB medium and was cultured at 37° C. and passaged twice to segregate away the pKD46 and to obtain an ampicillin-sensitive strain. The plasmid pCP20 was introduced into the ampicillin-sensitive strain, and ampicillin-resistant strains were again obtained. After culturing the obtained strains at 40° C., colony direct PCR was performed on the resulting strains to confirm the deletion of the kanamycin resistance gene from the length of the amplified band. Oligo DNA primers represented by SEQ IT) NOs: 224 and 225 were used. Subsequently, one of the kanamycin-sensitive strains was inoculated into 5 mL of LB medium and was cultured at 37° C. and passaged twice to segregate away the pCP20. The obtained strain was designated as Serratia grimestiNBRC13537 zlpyk⁻F.

Generation of a Mutant Microorganism of the Genus Serratia Deficient in pykA

A PCR reaction was performed using pKD4 as a template and oligo DNAs represented by SEQ ID NOs: 226 and 227 as primers to obtain a PCR fragment of 1.6 kb in length for disruption of pykA.

By the same method as used for the generation of the pykA-deficient strain, pykA was disrupted in the Serratia grimesii NBRC13537 zipykE strain. After the plasmid pKD46 was introduced into the above strain, the PCR fragment used for disruption of pykA was introduced to the resulting strain. Direct colony PCR was performed on the resulting kanamycin-resistant strains to confirm the deletion of the gene of interest and the insertion of a kanamycin resistance gene from the length of the amplified band. Oligo DNA primers represented by SEQ ID NOs: 223 and 229 were used.

Subsequently, an ampicillin-sensitive strain was obtained by segregating away the pKD46. The plasmid pCP20 was introduced into the ampicillin-sensitive strain, and ampicillin-resistant strains were again obtained. Colony direct PCR was performed on the obtained strains to confirm the deletion of the kanamycin resistance gene from the length of the amplified band. Oligo DNA primers represented by SEQ ID NOs: 228 and 229 were used. The pCP20 was segregated away from one of the kanamycin-sensitive strains. The obtained strain was designated as SgΔPP.

### Example 2

Generation of mutant microorganisms of the genus Serratia with impaired pyruvate kinase function and carrying a plasmid expressing enzymes that catalyze the reactions A, B, E, and F

Each of the plasmids produced in Reference Example 1 was introduced into the SgΔPP produced in Example 1 to generate mutant microorganisms of the genus Serratia. Additionally, a mutant microorganism of the genus Serratia was generated as a control by introducing the pBBR1MCS-2 empty vector into the SgΔPP.

The SgΔPP was inoculated into 5 mL of LB medium and cultured at 30° C. with shaking for I day. Subsequently, 0.5 mL of the culture fluid was inoculated into 5 mL of LB medium and was cultured at 30° C. with shaking for 2 hours. The culture fluid was cooled on ice for 20 minutes, and the bacterial cells were then washed with 10% (ww) glycerol three times. The washed pellet was suspended in 100 μL of 10% (ww) glycerol and mixed with 1 uL of the pBBR1MCS-2 (control), pBBR1MCS-2::ATCTOR1, pBBRIMCS-2::ATCTOR2, pBBR1MCS-2::ATCTOR3, pBBRIMCS-2::ATCTOR4, pBBR1MCS-2::ATCTOR5, pBBR1MCS-2::ATCTOR6, or pBBR1MCS-2::ATCTOR7, and the mixture was then cooled in an electroporation cuvette on ice for 10 minutes. Electroporation was performed using a Gene Pulser electroporator (manufactured by Bio-Rad Laboratories, Inc.; 3 kV, 200 Ω, 25 μF), and 1 mL of SOC medium was added to the electroporation cuvette immediately after the electroporation, and the bacterial cells in the cuvette were incubated at 30° C. with shaking for 1 hour. Fifty μL of the culture was applied to LB agar medium containing 25 μgmL kanamycin and was incubated at 30° C. for 1 day. The obtained strains were designated as SgΔPPpBBR (negative control), SgΔPP3HA1, SgΔPP31IA2, SgΔPP3HA3, SgΔPP3HA4, SgΔPP3HA5, SgΔPP3HA6, and SgΔPP3HA7.

### Reference Example 5

Generation of Mutant Microorganisms of the Genus Serratia with Intact Pyruvate Kinase Function and Carrying a Plasmid Expressing Enzymes that Catalyze the Reactions A, B, E, and F

By the same method as in Example 2, the pBBR1MCS-2 (control), pBBR1MCS-2::ATCTOR1, pBBR1MCS-2::ATCTOR2, pBBR1MCS-2::ATCTOR3, pBBRl S-2::ATCTOR4,)1313R1MCS-2::AICTOR5, pBBR1MCS-2::ATCTOR6, or pBBRlMCS-2::ATCTOR7 was introduced into Serratia grimesii NBRC13537. The obtained strains were designated as SgpBBR (negative control), Sg3HA1, Sg31-1A2, Sg3HA3, Sg3HA4, Sg3HA5, Sg3HA6, and Sg3HA7.

### Example 3

Production test of 3-hydroxyadipic acid and u-hydromuconic acid using mutant microorganisms of the genus Serratia with impaired pyruvate kinase function

The production test of 3-hydroxyadipic acid and α-hydromuconic acid was conducted using the mutant microorganisms of the genus Serratia produced in Example 2.

A loopful of each mutant produced in Example 2 was inoculated into 5 mL (in a glass test tube of 18-mm diameter with aluminum cap) of the culture medium 1 (10 gL Bacto Tryptone (manufactured by Difco Laboratories), 5 gL Bacto Yeast Extract (manufactured by Difco Laboratories), 5 gL sodium chloride, 25 μgmL kanamycin) adjusted to pH 7 and was cultured at 30° C. with shaking at 120 min⁻¹ for 24 hours. Subsequently, 0.25 mL of the culture fluid was added to 5 mI, (in a glass test tube of 18-mm diameter with aluminum cap) of the culture medium II (50gL glucose. 1 gL ammonium sulfate, 50 mM potassium phosphate, 0.025 gL magnesium sulfate, 0.0625 mgL iron sulfate, 2.7 mgL manganese sulfate, 0.33 mgL calcium chloride, 1.25 gL sodium chloride, 2.5 gL Bacto Tryptone. 1.25 gL, Bacto Yeast Extract, 25 μgmL kanamycin) adjusted to p11 6.5 and was cultured at 30° C. with shaking at 120 min^{-I} for 24 hours.

### Quantitative Analysis of Substrate and Product

The supernatant separated from bacterial cells by centrifugation of each culture fluid was processed by membrane treatment using Millex-GV (0.22 μm; PVDF; manufactured by Merck KGaA), and the resulting filtrate was analyzed by the following methods to quantify the concentrations of 3-hydroxyadipic acid, α-hydromuconic acid, and other products accumulated in the culture supernatant and the concentration of sugars remaining unused in the culture medium. The yields of 3-hydroxyadipic acid and α-hydromuconic acid calculated using the above formula (2) from the measurement results are shown in Table 7. However, a concentration of not more than 0.1 mgL is considered to be below the detection limit in the quantitative LC-MSMS analysis and is hereinafter denoted in each table as N.D.

### Quantitative Analysis of 3-Hydroxyadipic Acid and α-Hydromuconic Acid by LC-MSMS

HPLC: 1290 Infinity (manufactured by Agilent Technologies, Inc.)
Column: Synergi hydro-RP (manufactured by Phenomenex Inc.), length: 100 mm, internal diameter: 3 mm, particle size: 2.5 μm
Mobile phase: 0.1% aqueous formic acid solution methanol =7030
Flow rate: 0.3 mLmin
Column temperature: 40° C.
LC detector: 1260DAD VL+(210 nm)
MSMS: Triple-Quad LCMS (manufactured by Agilent Technologies, Inc.)
Ionization method: ESI in negative mode.
   Quantitative analysis of organic acids by HPLC
HPLC:LC-10A (manufactured by Shimadzu Corporation)
Column: Shim-pack SPR-H (manufactured by Shimadzu GLC Ltd.), length: 250 mm, internal diameter: 7.8 mm, particle size: 8 μm
Shim-pack SCR-101H (manufactured by Shimadzu GLC Ltd.) length: 250 mm, internal diameter: 7.8 mm, particle size: 10 um
Mobile phase: 5 mM p-toluenesulfonic acid
Reaction solution: 5 mMp-toluenesulfonic acid, 0.1 mM EDTA, 20 mM Bis-Tris
Flow rate: 0.8 mLmin
Column temperature: 45° C.
Detector: CDD-l0Avp (manufactured by Shimadzu Corporation)

### Quantitative Analysis of Sugars and Alcohol by HPLC

HPLC: Shimazu Prominence (manufactured by Shimadzu Corporation)
Column: Shodex Sugar SII41011 (manufactured by Showa Denko K.K.), length: 300 mm, internal diameter: 8 mm, particle size: 6 μm
Mobile phase: 0.05M aqueous sulfuric acid solution
Flow rate: 0.6 mLmin
Column temperature: 65° C.
Detector: RID-10A (manufactured by Shimadzu Corporation).

### Comparative Example 1

Production test of 3-hydroxyadipic acid and α-hydromuconic acid using mutant microorganisms of the genus Serratia with intact pyruvate kinase function

The mutant microorganisms of the genus Serratia produced in Reference Example 5 were cultured in the same manner as in Example 3. The concentrations of 3-hydroxyadipic acid, α-hydromuconic acid, and other products accumulated in the culture supernatant and the concentration of sugars remaining unused in the culture medium were quantified. The yields of 3-hydroxyadipic acid and α-hydromuconic acid calculated using the above formula (2) from the measurement results are shown in Table 7.

By comparing the results of Comparative Example 1 and Example 3, it was found that the yields of 3-hydroxyadipic acid and ct-hydromuconic acid were increased by impairing the function of pyruvate kinase in the microorganism of the genus Serratia.

TABLE 7

| | | Yield of | Yield of |
|---|---|---|---|
| | Strain | 3HA (%) | HMA (%) |
| Example 3 | SgΔPP/ | 0.0362 | 0.0113 |
| | pBBR | | |
| | SgΔPP/3HA1 | 3.47 | 0.0782 |
| | SgΔPP/3HA2 | 5.78 | 0.0960 |
| | SgΔPP/3HA3 | 5.24 | 0.0846 |
| | SgΔPP/3HA4 | 5.10 | 0.0909 |
| | SgΔPP/3HA5 | 6.21 | 0.107 |
| | SgΔPP/3HA6 | 6.28 | 0.103 |
| | SgΔPP/3HA7 | 4.96 | 0.0638 |
| Comparative | Sg/ | N.D. | N.D. |
| Example 1 | pBBR | | |
| | Sg/3HA1 | 0.784 | 0.0293 |
| | Sg/3HA2 | 1.15 | 0.0470 |
| | Sg/3HA3 | 0.942 | 0.0461 |
| | Sg/3HA4 | 0.875 | 0.0418 |
| | Sg/3HA5 | 1.01 | 0.0529 |
| | Sg/3HA6 | 1.03 | 0.0366 |
| | Sg/3HA7 | 0.943 | 0.0237 |

### Example 4

Generation of an E. coli mutant with impaired pyruvate kinase function

Genes encoding the pyruvate kinase of E. coli, pykF and pykA, were disrupted to generate an E. coli mutant with impaired pyruvate kinase function. The procedure for disrupting pykF and pykA followed the method described in Proc Natl Acad Sci USA., 2000 Jun. 6, 97(12): 6640-6645.

Generation of an E. Coli Mutant Deficient in pykF

A PCR reaction was performed using pKD4 as a template and oligo DNAs represented by SEQ ID NOs: 230 and 231 as primers to obtain a PCR fragment of 1.6 kb in length for disruption of pykF.

A FRT recombinase expression plasmid, pKD46, was introduced into Escherichia coli strain MG1655, and an ampicillin-resistant strain was obtained. The obtained strain was inoculated into 5 mL of LB medium containing 100 μgmL ampicillin and cultured at 30° C. with shaking for 1 day. Subsequently, 0.5 mL of the culture fluid was inoculated into 50 mL of LB medium containing 100 ugmL ampicillin and 50 mM arabinose, and was cultured in rotation at 30° C. for 2 hours. The culture fluid was cooled on ice for 20 minutes, and the bacterial cells were then washed with 10% (ww) glycerol three times. The washed pellet was suspended in 100 μL of 10% (ww) glycerol and mixed with 5 μL of the PCR fragment, and the mixture was then cooled in an electroporation cuvette on ice for 10 minutes. Electroporation was performed using a Gene Pulser electroporator (manufactured by Bio-Rad Laboratories, Inc.; 3 kV, 200 Q, 25 uF), and 1 mL of SOC medium was added to the electroporation cuvette immediately after the electroporation, and the bacterial cells in the cuvette were incubated at 30° C. with shaking for 2 hours. The total volume of the culture was applied to LB agar medium containing 25 μgmL kanamycin and was incubated at 30° C. for 1 day. Direct colony PCR was performed on the resulting kanamycin-resistant strains to confinn the deletion of the gene of interest and the insertion of a kanamycin resistance gene from the length of the amplified band. Oligo DNA primers represented by SEQ ID NOs: 223 and 233 were used.

Subsequently, one of the kanamycin-resistant strains was inoculated into 5 mL of LB medium and was cultured at 37° C. and passaged twice to segregate away the pKD46 and to obtain an ampicillin-sensitive strain. The plasmid pCP20 was introduced into the ampicillin-sensitive strain, and ampicillin-resistant strains were again obtained. After culturing the obtained strains at 40° C., direct colony PCR was performed on the resulting strains to confirm the deletion of the kanamycin resistance gene from the length of the amplified band. Oligo DNA primers represented by SEQ ID NOs: 232 and 233 were used. Subsequently, one of the kanamycin-sensitive strains was inoculated into 5 mL of LB medium and was cultured at 37° C. and passaged twice to segregate away the pCP20. The obtained strain was designated as Escherichia coli MG1655 zlpykF.

Generation of an E. coli Mutant Deficient in pykA

A PCR reaction was performed using pKD4 as a template and oligo DNAs represented by SEQ ID NOs: 234 and 235 as primers to obtain a PCR fragment of 1.6 kb in length for disruption of pykA.

By the same method as used for the generation of the pykF-deficient strain, pykA was disrupted in the Escherichia coli MG1655 zlpykF strain. After the plasmid pKD46 was introduced into the above strain, the PCR fragment used for disruption of pykA was introduced to the resulting strain. Direct colony PCR was performed on the resulting kanamycin-resistant strains to confirm the deletion of the gene of interest and the insertion of a kanamycin resistance gene from the length of the amplified band. Oligo DNA primers represented by SEQ ID NOs: 223 and 224 were used.

Subsequently, an ampicillin-sensitive strain was obtained by segregating away the pKD46. The plasmid pCP20 was introduced into the ampicillin-sensitive strain, and ampicillin-resistant strains were again obtained. Direct colony PCR was performed on the obtained strains to confirm the deletion of the kanamycin resistance gene from the length of the amplified band. Oligo DNA primers represented by SEQ ID NOs: 236 and 237 were used. The pCP20 was segregated away from one of the kanamycin-sensitive strains. The obtained strain was designated as EcΔPP.

### Example 5

Generation of E. coli Mutants with Impaired Pyruvate Kinase Function and Carrying a Plasmid Expressing Enzymes that Catalyze the Reactions A, B, E, and F

Each of the plasmids produced in Reference Example 1 was introduced into the EcΔPP produced in Example 4 to generate E. coli mutants.

The EcΔPP was inoculated into 5 mL of LB medium and cultured at 30° C. with shaking for 1 day. Subsequently, 0.5 mL of the culture fluid was inoculated into 5 mL of LB medium and was cultured at 30° C. with shaking for 2 hours. The culture fluid was cooled on ice for 20 minutes, and the bacterial cells were then washed with 10% (ww) glycerol three times. The washed pellet was suspended in 100 μL of 10% (wvv) glycerol and mixed with 1 μL of the pBBR1MCS-2 (negative control), pBBR1MCS-2::ATCTOR1, pBBR1MCS-2::ATCTOR2, pBBR1MCS-2::ATCTOR3. pBBR1MCS-2::ATCTOR4, pBBR1MCS-2::ATCTOR5, pBBR1MCS-2::ATCTOR6, or pBBR1MCS-2::ATCTOR7, and the mixture was then cooled in an electroporation cuvette on ice for 10 minutes. Electroporation was performed using a Gene Pulser electroporator (manufactured by Bio-Rad Laboratories, Inc.; 3 kV, 200 Q, 25 μF). and 1 mL of SOC medium was added to the electroporation cuvette immediately after the electroporation, and the bacterial cells in the cuvette were incubated at 30° C. with shaking for 1 hour. Fifty μL of the culture was applied to LB agar medium containing 25 pgmL kanamycin and was incubated at 30° C. for 1 day. The obtained strains were designated as EcΔPPpBBR (negative control), EcΔPP3HA1, EcΔPP3HA2, EcΔPP31-1A3, EcΔPP3HA4, EcΔPP3HA5, EcΔPP3HA6, and EcΔPP3IIA7.

### Reference Example 6

Generation of E. Coli Mutants with Intact Pyruvate Kinase Function and Carrying a Plasmid Expressing Enzymes that Catalyze the Reactions A, B, E, and F

By the same method as in Example 5, the pBBR1MCS-2 (control), pBBR1MCS-2::ATCTOR1, pBBR1MCS-2::ATCTOR2, pBBR1MCS-2::ATCTOR3, pBBR1MCS-2::ATCTOR4, pBBR1MCS-2::ATCTORS, pBBR1MCS-2::ATCTOR6, or pBBR1MCS-2::ATCTOR7 was introduced into Escherichia coli MG1655. The obtained strains were designated as EepBBR (negative control), Ec3IIA1, Ec3HA2, Ec3HA2, Ec3HA4, Ec3HA5, Ec3HA6, and Ec3HA7.

### Example 6

Production Test of 3-Hydroxyadipic Acid and α-Hydromuconic Acid Using E. Coli Mutants with Impaired Pyruvate Kinase Function

The mutants produced in Example 5 were cultured in the same manner as in Example 3. The concentrations of 3-hydroxyadipic acid, α-hydromuconic acid, and other products accumulated in the culture supernatant and the concentration of sugars remaining unused in the culture medium were quantified. The yields of 3-hydroxyadipic acid and α-hydromuconic acid calculated using the above formula (2) from the measured values are shown in Table 8.

### Comparative Example 2

Production Ttest of 3-Hydroxyadipic Acid and α-Hydromuconic Acid Using E. Coli Mutants with Intact Pyruvate Kinase Function

The mutants produced in Reference Example 6 were cultured in the same manner as in Example 6. The concentrations of 3-hydroxyadipic acid, α-hydromuconic acid, and other products accumulated in the culture supernatant and the concentration of sugars remaining unused in the culture medium were quantified. The yields of 3-hydroxyadipic acid and ci-hydromuconic acid calculated using the above formula (2) from the measured values are shown in Table 8.

By comparing the results of Comparative Example 2 and Example 6. it was found that the yields of 3-hydroxyadipic acid and 0.-hydromuconic acid were increased by impairing the function of pyruvate kinase in E. coli.

TABLE 8

| | | Yield of | Yield of |
|---|---|---|---|
| | Strain | 3HA (%) | HMA (%) |
| Example 6 | EcΔPP/ | 0.0427 | 0.0132 |
| | pBBR | | |
| | EcΔPP/3HA1 | 2.54 | 0.0292 |
| | EcΔPP/3HA2 | 3.97 | 0.0333 |
| | EcΔPP/3HA3 | 3.64 | 0.0273 |
| | EcΔPP/3HA4 | 2.86 | 0.0257 |
| | EcΔPP/3HA5 | 3.67 | 0.0269 |
| | EcΔPP/3HA6 | 3.57 | 0.0348 |
| | EcΔPP/3HA7 | 3.13 | 0.0274 |
| Comparative | Ec/ | N.D. | N.D. |
| Example 2 | pBBR | | |
| | Ec/3HA1 | 1.48 | 0.0172 |
| | Ec/3HA2 | 2.59 | 0.0160 |
| | Ec/3HA3 | 2.64 | 0.0167 |
| | Ec/3HA4 | 1.82 | 0.0186 |
| | Ec/3HA5 | 2.47 | 0.0166 |
| | Ec/3HA6 | 2.66 | 0.0228 |
| | Ec/3HA7 | 1.78 | 0.0172 |

### Example 7

Generation of mutant microorganisms of the genus Serratia with impaired pyruvate kinase function and carrying plasmids expressing enzymes that catalyze the reactions A, B, C, E, and F

The plasmid pMW119::EH produced in Reference Example 2 was introduced into each mutant microorganism of the genus Serratia produced in Example 2 to generate mutant microorganisms of the genus Serratia . Additionally, a mutant microorganism of the genus Serratia was generated as a control by introducing the pMW119 empty vector into the SgΔPPpBBR produced in Example 2.

The SgΔPPpBBR, SgΔPP3HA1 SgΔPP3HA2, SgΔPP3HA3, SgΔPP3HA4, SgΔPP3HA5, SgΔPP3HA6, or SgΔPP3HA7 was inoculated into 5 mi. of LB medium containing 25 μgmL kanamycin and cultured at 30° C. with shaking for 1 day. Subsequently, 0.5 mL of the culture fluid was inoculated into 5 mf. of LB medium containing 25 μgmL kanamycin and was cultured at 30° C. with shaking for 2 hours. The culture fluid was cooled on ice for 20 minutes, and the bacterial cells were then washed with 10% (ww) glycerol three times. The washed pellet was suspended in 100 μL of 10% (ww) glycerol and mixed with 1 μL of the pBBR1MCS-2 (control) or pMW119::EH, and the mixture was then cooled in an electroporation cuvette on ice for 10 minutes. Electroporation was performed using a Gene Pulser electroporator (manufactured by Bio-Rad Laboratories, Inc.; 3 kV, 200 Ω, 25 μF), and 1 mL of SOC medium was added to the electroporation cuvette immediately after the electroporation, and the bacterial cells in the cuvette were incubated at 30° C. with shaking for 1 hour. Fitly of the culture was applied to LB agar medium containing 500 ugmL ampicillin and 25 μgmL kanamycin and was incubated at 30° C. for 1 day. The obtained strains were designated as SgΔPPpBBRpMW (negative control), SgΔPPIIMA1, SgΔPPHMA2, SgΔPPHMA3, SgΔPPHMA4, SgΔPPHMAS, SgΔPPHMA6, and S APPHMA7.

### Reference Example 7

Generation of mutant microorganisms of the genus Serratia with intact pyruvate kinase function and carrying plasmids expressing enzymes that catalyze the reactions A, B, C, E, and F

By the same method as in Example 7, the pMW119 (control) or pMW119::EH was introduced into SgpBBR, Sg3HA1, Sg3HA2, Sg3HA3, Sg3HA4, Sg3HA5, Sg311A6, and Sg3HA7. The obtained strains were designated as SgpBBRpMW (negative control), SgHMA1, SgHMA2, SgHMA3, SgHMA4, SgHMA5, SgHMA6, and SgHMA7.

### Example 8

Production test of α-hydromuconic acid using mutant microorganisms of the genus Semliki with impaired pyruvate kinase function

The mutants produced in Example 7 were cultured in the same manner as in Example 3, except that ampicillin was added to the culture medium to a final concentration of 500 μgmL. The concentrations of α-hydromuconic acid and other products accumulated in the culture supernatant and the concentration of sugars remaining unused in the culture medium were quantified. The yield of α-hydromuconic acid calculated using the above formula (2) from the measured values is shown in Table 9.

### Comparative Example 3

Production test of α-hydromuconic acid using mutant microorganisms of the genus Serratia with intact pyruvate kinase function

The mutants produced in Reference Example 7 were cultured in the same manner as in Example 8. The concentrations of α-hydromuconic acid and other products accumulated in the culture supernatant and the concentration of sugars remaining unused in the culture medium were quantified. The yield of α-hydromuconie acid calculated using the above formula (2) from the measured values is shown in Table 9.

By comparing the results of Comparative Example 3 and Example 8, it was found that the yield of a-hydromuconic acid was increased by impairing the function of pyruvate kinase in the microorganism of the genus Serratia.

TABLE 9

| | | Yield of |
|---|---|---|
| | Strain | HMA (%) |
| Example 8 | SgΔPP/ | 0.0119 |
| | pBBRpMW | |
| | SgΔPP/HMA1 | 0.156 |
| | SgΔPP/HMA2 | 0.179 |
| | SgΔPP/HMA3 | 0.153 |
| | SgΔPP/HMA4 | 0.118 |
| | SgΔPP/HMA5 | 0.217 |
| | SgΔPP/HMA6 | 0.241 |
| | SgΔPP/HMA7 | 0.140 |
| Comparative | Sg/ | N.D. |
| Example 3 | pBBRpMW | |
| | Sg/HMA1 | 0.0495 |
| | Sg/HMA2 | 0.0584 |
| | Sg/HMA3 | 0.0434 |
| | Sg/HMA4 | 0.0524 |
| | Sg/HMA5 | 0.0587 |
| | Sg/HMA6 | 0.0618 |
| | Sg/HMA7 | 0.0519 |

### Example 9

Generation of E. coli mutants with impaired pyruvate kinase function and carrying plasmids expressing enzymes that catalyze the reactions A, B, C, E, and F

The plasmid pMW119::EH produced in Reference Example 2 was introduced into each of the E. coli mutants produced in Example 5 to generate E. coli mutants. Additionally, an E. coli mutant was generated as a control by introducing the pMW119 empty vector into the EcΔPPpBBR produced in Example 5.

The EcΔPPpBBR. EcΔPP311⁻A1, EcΔPPRHA2, EcΔPP3HA3, EcΔPP3HA4. EcΔPP3HA5, EcΔPP311A6, or EcΔPP3HA7 was inoculated into 5 mL of LB medium containing 25 μgmL kanamycin and cultured at 30° C. with shaking for 1 day. Subsequently, 0.5 mL of the culture fluid was inoculated into 5 mL of LB medium containing 25 μgmL kanamycin and was cultured at 30° C. with shaking for 2 hours. The culture fluid was cooled on ice for 20 minutes, and the bacterial cells were then washed with 10% (ww) glycerol three times. The washed pellet was suspended in 100 μL of 10% (ww) glycerol and mixed with 1 uL of the pBBR1MCS-2 (control) or pMW1 19::EH, and the mixture was then cooled in an electroporation cuvette on ice for 10 minutes. Electroporation was performed using a Gene Pulser electroporator (manufactured by Bio-Rad Laboratories, Inc.; 3 kV, 200 Ω, 25 μF.), and 1 mL of SOC medium was added to the electroporation cuvette immediately after the electroporation, and the bacterial cells in the cuvette were incubated at 30° C. with shaking for 1 hour. Fifty uL of the culture was applied to LB agar medium containing 100 μgmL ampicillin and 25 μgmL kanamycin and was incubated at 30° C. for 1 day. The obtained strains were designated as EcΔPPpBBRpMW (negative control), EcΔPPHMA1, EcΔPPHMA2, EcΔPPHMA3, EcΔPPHMA4 EcΔPPHMAS, EcΔPPHMA6, and EcΔPPHMA7.

### Reference Example 8

Generation of E. coli mutants with intact pyruvate kinase function and carrying plasmids expressing enzymes that catalyze the reactions A, B, C, E, and F

By the same method as in Example 9, the pMW119 (control) or pMW119::EH was introduced into the EcpBBR, Ec3HA1, Ec3HA2, Ec31IA3, Ec3HA4, Ec3HA5, Ec3HA6, and Ec3HA7. The obtained strains were designated as EcpBBRpMW (negative control), EcHMA1, EcHMA2, EcIIMA3, EcHMA4, EcHMAS, EcIIMA6. and EcHMA7.

### Example 10

Production test of α-hydromuconic acid using E. coli mutants with impaired pyruvate kinase (Unction

The mutants produced in Reference Example 9 were cultured in the same manner as in Example 6, except that ampicillin was added to the culture medium to a concentration of 100 μgmL. The concentrations of α-hydromuconic acid and other products accumulated in the culture supernatant and the concentration of sugars remaining unused in the culture medium were quantified. The yield of α-hydromuconic acid calculated using the above formula (2) from the measured values is shown in Table 10.

### Comparative Example 4

Production test of α-hydromuconic acid using E. coli mutants with intact pyruvate kinase function

The mutants produced in Reference Example 8 were cultured in the same manner as in Example 10. The concentrations of α-hydromuconic acid and other products accumulated in the culture supernatant and the concentration of sugars remaining unused in the culture medium were quantified. The yield of α-hydromuconic acid calculated using the above formula (2) from the measured values is shown in Table 10.

By comparing the results of Comparative Example 4 and Example 10, it was found that the yield of α-hydromuconic acid was increased by impairing the function of pyruvate kinase in E. coli.

TABLE 10

| | | Yield of |
|---|---|---|
| | Strain | HMA (%) |
| Example 10 | EcΔPP/ | 0.0167 |
| | pBBRpMW | |
| | EcΔPP/HMA1 | 0.0511 |
| | EcΔPP/HMA2 | 0.0818 |
| | EcΔPP/HMA3 | 0.0717 |
| | EcΔPP/HMA4 | 0.0688 |
| | EcΔPP/HMA5 | 0.0765 |
| | EcΔPP/HMA6 | 0.0761 |
| | EcΔPP/HMA7 | 0.0599 |
| Comparative | Ec/ | N.D. |
| Example 4 | pBBRpMW | |
| | Ec/HMA1 | 0.0362 |
| | Ec/HMA2 | 0.0636 |
| | Ec/HMA3 | 0.0569 |
| | Ec/HMA4 | 0.0624 |
| | Ec/HMA5 | 0.0621 |
| | Ec/HMA6 | 0.0640 |
| | Ec/HMA7 | 0.0491 |

### Example 11

Generation of mutant microorganisms of the genus Serratia with impaired pyruvate kinase function and carrying plasmids expressing enzymes that catalyze the reactions A, B. C, D, and G

By the same method as in Example 2, the pBBR1MCS-2::ATCT2OR1, pBBR1MCS-2::ATCT2OR2, pBBR1MCS-2::ATCT2OR3, pBBR1.MCS-2::ATCT2OR4, pBBR1MCS-2::ATCT2OR5, pBBR1MCS-2::ATCT2OR6, or pBBR1MCS-2::ATCT2OR7 produced in Reference Example 3 was introduced into the SgΔPP. By the same method as in Example 7, the plasmid pMW119::EIIER produced in Reference Example 4 was introduced into each of the obtained mutants to generate mutant microorganisms of the genus Serratia . The obtained strains were designated as SgΔPPADA1, SgΔPPADA2, SgΔPPADA3, SgΔPPADA4, SgΔPPADAS, SgΔPPADA6, and SgΔPPADA7.

### Reference Example 9

Generation of mutant microorganisms of the genus Serratia with intact pyruvate kinase function and carrying plasmids expressing enzymes that catalyze the reactions A, B, C, D, and G

By the same method as in Example 11, the pBBR1MCS-2::ATCT2OR1, pBBR1MCS-2::ATCT2OR2, pBBR1MCS-2::ATCT2OR3, pBBR1MCS-2::ATCT2OR4, pBBR1MCS-2::ATCT2OR5, pBBR1MCS-2::ATCT2OR6, or pBBR1MCS-2::ATCT2OR7 produced in Reference Example 3 was introduced into Serratia grimesii NBRC13537. By the same method as in Example 7, the plasmid pMW119::EHER produced in Reference Example 4 was introduced into each of the obtained mutants to generate mutant microorganisms of the genus Serratia . The obtained strains were designated as SgADA1, SgADA2, SgADA3, SgADA4, SgADAS, SgADA6, and SgADA7.

### Example 12

Production test of adipic acid using mutant microorganisms of the genus Serratia with impaired pyruvate kinase function

The mutants produced in Example 11 and the SgΔPPpBBRpMW (negative control) were cultured in the same manner as in Example 8. The concentrations of adipic acid and other products accumulated in the culture supernatant and the concentration of sugars remaining unused in the culture medium were quantified. The quantification of adipic acid was performed using LC-MSMS under the same conditions for the quantification of 3-hydroxyadipic acid and α-hydromuconic acid. The yield of adipic acid calculated using the above formula (2) from the measured values is shown in Table 11.

### Comparative Example 5

Production test of adipic acid using mutant microorganisms of the genus Serratia with intact pyruvate kinase function

The mutants produced in Reference Example 9 and the SgpBBRpMW were cultured in the same manner as in Example 8. The concentrations of adipic acid and other products accumulated in the culture supernatant and the concentration of sugars remaining unused in the culture medium were quantified. The yield of adipic acid calculated using the above formula (2) from the measured values is shown in Table 11.

By comparing the results of Comparative Example 5 and Example 12, it was found that the yield of adipic acid was increased by impairing the function of pyruvate kinase in the microorganism of the genus Serratia .

TABLE 11

| | | Yield of |
|---|---|---|
| | Strain | ADA (%) |
| Example 12 | SgΔPP/ | N.D. |
| | pBBRpMW | |
| | SgΔPP/ADA1 | 0.0783 |
| | SgΔPP/ADA2 | 0.110 |
| | SgΔPP/ADA3 | 0.0861 |
| | SgΔPP/ADA4 | 0.116 |
| | SgΔPP/ADA5 | 0.108 |
| | SgΔPP/ADA6 | 0.136 |
| | SgΔPP/ADA7 | 0.0958 |
| Comparative | Sg/ | N.D. |
| Example 5 | pBBRpMW | |
| | Sg/ADA1 | 0.0244 |
| | Sg/ADA2 | 0.0325 |
| | Sg/ADA3 | 0.0254 |
| | Sg/ADA4 | 0.0246 |
| | Sg/ADA5 | 0.0314 |
| | Sg/ADA6 | 0.0264 |
| | Sg/ADA7 | 0.0289 |

### Example 13

Generation of E. coli mutants with impaired pyruvate kinase function and carrying plasmids expressing enzymes that catalyze the reactions A, B, C, D, and G

By the same method as in Example 5, the PBBR1MCS-2::ATCT2OR1, pBBR1MCS-2::ATCT2OR2, pBBR1MCS-2::ATCT2OR3, pBBR1MCS-2::ATCT2OR4, pBBR1MCS-2::ATCT2OR5, pBBR1MCS-2::ATCT2OR6, or pBBR1MCS-2::ATCT2OR7 produced in Reference Example 3 was introduced into the EeAPP. By the same method as in Example 9, the plasmid pMW119::EHER produced in Reference Example 4 was introduced into each of the obtained mutants to generate E. coli mutants. The obtained strains were designated as EcΔPPADA1, EcΔPPADA2, EcΔPPADA3, EcΔPPADA4, EcΔPPADAS, EcΔPPADA6, and EcΔPPADA7.

### Reference Example 10

Generation of E. coli mutants with intact pyruvate kinase function and carrying plasmids expressing enzymes that catalyze the reactions A, B, C, D, and G

By the same method as in Example 13, the pBBR1MCS-2::ATCT2OR1, pBBR1MCS-2::ATCT2OR2, pBBR1MCS-2::ATCT2OR3, pBBR1MCS-2::ATCT2OR4, pBBR1MCS-2::ATCT2OR5, pBBR1MCS-2::ATCT2OR6, or pBBR1MCS-2::ATCT2OR7 produced in Reference Example 3 was introduced into Escherichia coli MG1655. By the same method as in Example 9, the plasmid pMW119::EHER produced in Reference Example 4 was introduced into each of the obtained mutants to generate E. coli mutants. The obtained strains were designated as EcADA1, EcADA2, EcADA3, EcADA4, EcADAS, EcADA6, and EcADA7.

### Example 14

Production test of adipic acid using E. coli mutants with impaired pyruvate kinase function

The mutants produced in Example 13 and the EcΔPPpBBRpMW were cultured in the same manner as in Example 10. The concentrations of adipic acid and other products accumulated in the culture supernatant and the concentration of sugars remaining unused in the culture medium were quantified. The quantification of adipic acid was performed using LC-MSMS under the same conditions for the quantification of 3-hydroxyadipic acid and α-hydromuconic acid. The yield of adipic acid calculated using the above formula (2) from the measured values is shown in Table 12.

### Comparative Example 6

Production test of adipic acid using E. coli mutants with intact pyruvate kinase function

The mutants produced in Reference Example 10 and the EcpBBRpMW were cultured in the same manner as in Example 10. The concentrations of adipic acid and other products accumulated in the culture supernatant and the concentration of sugars remaining unused in the culture medium were quantified. The yield of adipic acid calculated using the above formula (2) from the measured values is shown in Table 12.

By comparing the results of Comparative Example 6 and Example 14, it was found that the yield of adipic acid was increased by impairing the function of pyruvate kinase in E. coli.

TABLE 12

| | | Yield of |
|---|---|---|
| | Strain | ADA (%) |
| Example 14 | EcΔPP/ | N.D. |
| | pBBRpMW | |
| | EcΔPP/ADA1 | 0.0213 |
| | EcΔPP/ADA2 | 0.0338 |
| | EcΔPP/ADA3 | 0.0293 |
| | EcΔPP/ADA4 | 0.0315 |
| | EcΔPP/ADA5 | 0.0382 |
| | EcΔPP/ADA6 | 0.0407 |
| | EcΔPP/ADA7 | 0.0235 |
| Comparative | Ec/ | N.D. |
| Example 6 | pBBRpMW | |
| | Ec/ADA1 | 0.0148 |
| | Ec/ADA2 | 0.0153 |
| | Ec/ADA3 | 0.0107 |
| | Ec/ADA4 | 0.0192 |
| | Ec/ADA5 | 0.0139 |
| | Ec/ADA6 | 0.0147 |
| | Ec/ADA7 | 0.0167 |

### Example 15

Production test 2 of 3-hydroxyadipic acid and α-hydromuconic acid using mutant microorganisms of the genus Serratia with impaired pyruvate kinase function

The production test of 3-hydroxyadipic acid and a.-hydromuconic acid was conducted using the mutant microorganisms of the genus Serratia produced in Example 2 under anaerobic conditions.

The mutant microorganisms of the genus Serratia produced in Example 2 were cultured in the same manner as in Example 3, except that the mutant microorganisms were cultured statically using the culture medium II. The concentrations of 3-hydroxyadipic acid, α-hydromuconic acid, and other products accumulated in the culture supernatant and the concentration of sugars remaining unused in the culture medium were quantified. The yields of 3-hydroxyadipic acid and α-hydromuconic acid calculated using the above formula (2) from the measured values are shown in Table 13.

### Comparative Example 7

Production test 2 of 3-hydroxyadipic acid and α-hydromuconic acid using mutant microorganisms of the genus Serratia with intact pyruvate kinase function

The mutants produced in Reference Example 5 were cultured in the same manner as in Example 15. The concentrations of 3-hydroxyadipic acid, α-hydromuconic acid, and other products accumulated in the culture supernatant and the concentration of sugars remaining unused in the culture medium were quantified.

The yields of 3-hydroxyadipic acid and α-hydromuconic acid calculated using the above formula (2) from the measured values are shown in Table 13.

By comparing the results of Comparative Example 7 and Example 15, it was found that the yields of 3-hydroxyadipic acid and α-hydromuconic acid were increased even under anaerobic conditions by impairing the function of pyruvate kinase in the microorganism of the genus Serratia .

TABLE 13

| | | Yield of | Yield of |
|---|---|---|---|
| | Strain | 3HA (%) | HMA (%) |
| Example 15 | SgΔPP/ | 0.0485 | 0.0224 |
| | pBBR | | |
| | SgΔPP/3HA1 | 4.84 | 0.159 |
| | SgΔPP/3HA2 | 6.07 | 0.171 |
| | SgΔPP/3HA3 | 5.99 | 0.143 |
| | SgΔPP/3HA4 | 5.30 | 0.195 |
| | SgΔPP/3HA5 | 5.84 | 0.180 |
| | SgΔPP/3HA6 | 6.02 | 0.202 |
| | SgΔPP/3HA7 | 5.98 | 0.160 |
| Comparative | Sg/ | N.D. | N.D. |
| Example 7 | pBBR | | |
| | Sg/3HA1 | 1.68 | 0.0482 |
| | Sg/3HA2 | 2.46 | 0.0577 |
| | Sg/3HA3 | 1.94 | 0.0471 |
| | Sg/3HA4 | 1.99 | 0.0527 |
| | Sg/3HA5 | 2.29 | 0.0523 |
| | Sg/3HA6 | 2.95 | 0.0627 |
| | Sg/3HA7 | 1.66 | 0.0595 |

### Example 16

Production test 2 of 3-hydroxyadipic acid and u-hydromuconic acid using E. coli mutants with impaired pyruvate kinase function

The production test of 3-hydroxyadipic acid and u-hydromuconic acid was conducted under anaerobic conditions using the K coli mutants produced in Example 5.

The E. coli mutants produced in Example 5 were cultured in the same manner as in Example 6, except that the mutants were cultured statically using, the culture medium II. The concentrations of 3-hydroxyadipic acid, α-hydromuconic acid, and other products accumulated in the culture supernatant and the concentration of sugars remaining unused in the culture medium were quantified. The yields of 3-hydroxyadipic acid and α-hydromuconic acid calculated using the above formula (2) from the measured values are shown in Table 14.

### Comparative Example 8

Production test 2 of 3-hydroxyadipic acid and α-hydromuconic acid using E. coli mutants with intact pyruvate kinase function

The mutants produced in Reference Example 6 were cultured in the same manner as in Example 16. The concentrations of 3-hydroxyadipic acid, α-hydromuconic acid, and other products accumulated in the culture supernatant and the concentration of sugars remaining unused in the culture medium were quantified. The yields of 3-hydroxyadipic acid and α-hydromuconic acid calculated using the above formula (2) from the measured values are shown in Table 14.

By comparing the results of Comparative Example 8 and Example 16, it was found that the yields of 3-hydroxyadipic acid and α-hydromuconic acid were increased even under anaerobic conditions by impairing the function of pyruvate kinase in E. coli.

TABLE 14

| | | Yield of | Yield of |
|---|---|---|---|
| | Strain | 3HA (%) | HMA (%) |
| Example 16 | EcΔPP/ | 0.0669 | 0.0113 |
| | pBBR | | |
| | EcΔPP/3HA1 | 13.2 | 0.0213 |
| | EcΔPP/3HA2 | 14.9 | 0.0277 |
| | EcΔPP/3HA3 | 13.9 | 0.0268 |
| | EcΔPP/3HA4 | 14.1 | 0.0224 |
| | EcΔPP/3HA5 | 14.3 | 0.0259 |
| | EcΔPP/3HA6 | 14.7 | 0.0226 |
| | EcΔPP/3HA7 | 13.2 | 0.0213 |
| Comparative | Ec/ | N.D. | N.D. |
| Example 8 | pBBR | | |
| | Ec/3HA1 | 1.32 | 0.0171 |
| | Ec/3HA2 | 2.00 | 0.0154 |
| | Ec/3HA3 | 1.82 | 0.0130 |
| | Ec/3HA4 | 1.47 | 0.0136 |
| | Ec/3HA5 | 2.17 | 0.0138 |
| | Ec/3HA6 | 1.77 | 0.0166 |
| | Ec/3HA7 | 1.14 | 0.0179 |

### Example 17

Production test 2 of adipic acid using mutant microorganisms of the genus Serratia with impaired pyruvate kinase function

The production test of adipic acid was conducted using the mutant microorganisms of the genus Serratia produced in Example 11 under anaerobic conditions.

The mutant microorganisms of the genus Serratia produced in Example 11 were cultured in the same manner as in Example 12. except that the mutant microorganisms were cultured statically using the culture medium II. The concentrations of adipic acid and other products accumulated in the culture supernatant and the concentration of sugars remaining unused in the culture medium were quantified. The yield of adipic acid calculated using the above formula (2) from the measured values is shown in Table 15.

### Comparative Example 9

Production test 2 of adipic acid using mutant microorganisms of the genus Serratia with intact pyruvate kinase function

The mutants produced in Reference Example 9 were cultured in the same manner as in Example 17. The concentrations of adipic acid and other products accumulated in the culture supernatant and the concentration of sugars remaining unused in the culture medium were quantified. The yield of adipic acid calculated using the above formula (2) from the measured values is shown in Table 15.

By comparing the results of Comparative Example 9 and Example 17, it was found that the yield of adipic acid was increased even under anaerobic conditions by impairing the function of pyruvate kinase in the microorganism of the genus Serratia .

TABLE 15

| | | Yield of |
|---|---|---|
| | Strain | ADA (%) |
| Example 17 | SgΔPP/ | N.D. |
| | pBBRpMW | |
| | SgΔPP/ADA1 | 0.0359 |
| | SgΔPP/ADA2 | 0.0480 |
| | SgΔPP/ADA3 | 0.0379 |
| | SgΔPP/ADA4 | 0.0395 |
| | SgΔPP/ADA5 | 0.0431 |
| | SgΔPP/ADA6 | 0.0490 |
| | SgΔPP/ADA7 | 0.0375 |
| Comparative | Sg/ | N.D. |
| Example 9 | pBBRpMW | |
| | Sg/ADA1 | 0.0152 |
| | Sg/ADA2 | 0.0181 |
| | Sg/ADA3 | 0.0188 |
| | Sg/ADA4 | 0.0179 |
| | Sg/ADA5 | 0.0135 |
| | Sg/ADA6 | 0.0130 |
| | Sg/ADA7 | 0.0093 |

### Example 18

Production test 2 of adipic acid using E. coli mutants with impaired pyruvate kinase function

The production test of adipic acid was conducted using the E. coli mutants produced in Example 13 under anaerobic conditions.

The E. coli mutants produced in Example 13 were cultured in the same manner as in Example 14, except that the mutants were cultured statically using the culture medium II. The concentrations of adipic acid and other products accumulated in the culture supernatant and the concentration of sugars remaining unused in the culture medium were quantified. The yield of adipic acid calculated using the above formula (2) from the measured values is shown in Table 16.

### Comparative Example 10

Production test 2 of adipic acid using E. coli mutants with intact pyruvate kinase function

The mutants produced in Reference Example 10 were cultured in the same manner as in Example 18. The concentrations of adipic acid and other products accumulated in the culture supernatant and the concentration of sugars remaining unused in the culture medium were quantified. The yield of adipic acid calculated using the above formula (2) from the measured values is shown in Table 16.

By comparing the results of Comparative Example 10 and Example 18, it was found that the yield of adipic acid was increased even under anaerobic conditions by impairing the function of pyruvate kinase in E. coli.

TABLE 16

| | | Yield of |
|---|---|---|
| | Strain | ADA (%) |
| Example 18 | EcΔPP/ | N.D. |
| | pBBRpMW | |
| | EcΔPP/ADA1 | 0.0255 |
| | EcΔPP/ADA2 | 0.0254 |
| | EcΔPP/ADA3 | 0.0269 |
| | EcΔPP/ADA4 | 0.0248 |
| | EcΔPP/ADA5 | 0.0212 |
| | EcΔPP/ADA6 | 0.0278 |
| | EcΔPP/ADA7 | 0.0212 |
| Comparative | Ec/ | N.D. |
| Example 10 | pBBRpMW | |
| | Ec/ADA1 | 0.0166 |
| | Ec/ADA2 | 0.0180 |
| | Ec/ADA3 | 0.0140 |
| | Ec/ADA4 | 0.0161 |
| | Ec/ADA5 | 0.0148 |
| | Ec/ADA6 | 0.0219 |
| | Ec/ADA7 | 0.0123 |

### Example 19

Generation of a mutant microorganism of the genus Serratia with defects in genes encoding pyruvate kinase and a phosphotransferase system enzyme

A mutant microorganism of the genus Serratia with impaired function of both pyruvate kinase and a phosphotransferase system enzyme was generated by disrupting a gene encoding a phosphotransferase, ptsG, in the SgΔPP strain produced in Example 1.

A PCR reaction was performed using pKD4 as a template and oligo DNAs represented by SEQ ID NOs: 239 and 240 as primers to obtain a PCR fragment of 1.6 kb in length for disruption of ptsG. The introduction of pKD46 into the above strain was followed by the introduction of the PCR fragment for disruption of ptsG into the resulting strain. Direct colony PCR was performed on the resulting kanamycin-resistant strains to confirm the deletion of the gene of interest and the insertion of a kanamycin resistance gene from the length of the amplified band. Oligo DNA primers represented by SEQ ID NOs: 223 and 242 were used.

Subsequently, an ampicillin-sensitive strain was obtained by segregating away the pKD46. The plasmid pCP20 was introduced into the ampicillin-sensitive strain, and ampicillin-resistant strains were again obtained. Direct colony PCR was performed on the obtained strains to confirm the deletion of the kanamycin resistance gene from the length of the amplified band. Oligo DNA primers represented by SEQ ID NOs: 241 and 242 were used. The pCP20 was segregated away from one of the kanamycin-sensitive strains. The obtained strain is hereinafter referred to as SgΔPPG.

### Example 20

Generation of a mutant microorganism of the genus Serratia with defects in genes encoding pyruvate kinase and a phosphotransferase system enzyme and carrying a plasmid expressing enzymes that catalyze the reactions A, B, E, and F By the same method as in Example 2, a plasmid produced in Reference Example 1, pBBR1MCS-2::ATCTOR1, was introduced into the SgΔPPG strain produced in Example 19, and the obtained mutant microorganism of the genus Serratia was designated as SgΔPPG3HA1.

### Example 21

Production test of 3-hydroxyadipic acid and α-hydromuconic acid using a mutant microorganism of the genus Serratia with impaired function of both pyruvate kinase and a phosphotransferase system enzyme and carrying a plasmid expressing enzymes that catalyze the reactions A, B, E, and F

By the same method as in Example 15, the production test of 3-hydroxyadipic acid and α-hydromuconic acid was conducted using the mutant microorganism of the genus Serratia produced in Example 20.

### Comparative Example 11

Production test of 3-hydroxyadipic acid and α-hydromuconic acid using a mutant microorganism of the genus Serratia with intact pyruvate kinase function and intact phosphotransferase system enzyme function and carrying a plasmid expressing enzymes that catalyze the reactions A, B. E, and F

By the same method as in Comparative Example 7, the production test of 3-hydroxyadipic acid and α-hydromuconic acid was conducted using the Sg314A1 strain produced in Reference Example 5.

By comparing the results of Example 21 and Example 15, it was found that the yields of 3-hydroxyadipic acid and α-hydromuconic acid were further increased in the mutant microorganism of the genus Serratia with defects in the genes encoding pyruvate kinase and the phosphotransferase system enzyme and with enhanced activity of an enzyme that catalyzes the reaction of reducing 3-oxoadipyl-CoA to 3-hydroxyadipyl-CoA. Additionally, by comparing the results of Example 21 and Comparative Example 11, it was found that the yields of acetic acid and ethanol, both of which were generated by conversion of acetyl-CoA, were also increased in the mutant with defects in the genes encoding pyruvate kinase and the phosphotransferase system enzyme.

TABLE 17

| | | | | Yield of | Yield of | |
|---|---|---|---|---|---|---|
| | | Yield of | Yield of | succinic | acetic | Yield of |
| | Strain | 3HA (%) | HMA (%) | acid (%) | acid (%) | ethanol (%) |
| Example 21 | SgΔPPG/3HA1 | 6.06 | 0.180 | 60.6 | 36.8 | 52.2 |
| Comparative | Sg/3HA1 | 1.68 | 0.0482 | 7.26 | 35.1 | 38.8 |
| Example 11 | | | | | | |

### Example 22

Generation of an E. coli mutant with defects in genes encoding pyruvate kinase and a phosphotransferase system enzyme

An E. coli mutant with impaired function of both pyruvate kinase and a phosphotransferase system enzyme was generated by disrupting a gene encoding a phosphotransferase,ptsG, in the EcΔPP produced in Example 4.

A PCR reaction was performed using pKD4 as a template and oligo DNAs represented by SEQ ID NOs: 243 and 244 as primers to obtain a PCR fragment of 1.6 kb in length for disruption of ptsG. The introduction of pKD46 into the above strain was followed by the introduction of the PCR fragment for disruption of ptsG into the resulting strain. Direct colony PCR was performed on the resulting kanamycin-resistant strains to confirm the deletion of the gene of interest and the insertion of a kanamycin resistance gene from the length of the amplified band. Oligo DNA primers represented by SEQ ID NOs: 223 and 246 were used.

Subsequently, an ampicillin-sensitive strain was obtained by segregating away the pKD46. The plasmid pCP20 was introduced into the ampicillin-sensitive strain, and ampicillin-resistant strains were again obtained. Direct colony PCR was performed on the obtained strains to confirm the deletion of the kanamycin resistance gene :from the length of the amplified band. Oligo DNA primers represented by SEQ ID NOs: 245 and 246 were used. The pCP20 was segregated away from one of the kanamycin-sensitive strains. The obtained strain is hereinafter referred to as EcΔPPG.

### Example 23

Generation of an E. coli mutant with defects in genes encoding pyruvate kinase and a phosphotransferase system enzyme and carrying a plasmid expressing enzymes that catalyze the reactions A, B, E, and F

By the same method as in Example 5, the pBBIUMCS-2::ATCTOR1 produced in Reference Example 1 was introduced into the EcΔPPG strain produced in Example 22, and the obtained E. coli mutant was designated as EcΔPPG3HA1.

### Example 24

Production test of 3-hydroxyadipic acid and u-hydromuconic acid using an E. coli mutant with impaired function of both pyruvate kinase and a phosphotransferase system enzyme and carrying a plasmid expressing enzymes that catalyze the reactions A, B, E, and F

By the same method as in Example 16, the production test of 3-hydroxyadipic acid and α-hydromuconic acid was conducted using the E. coli mutant produced in Example 23.

### Comparative Example 12

Production test of 3-hydroxyadipic acid and α-hydromuconic acid using an E. coli mutant with intact pyruvate kinase function and intact phosphotransferase system enzyme function and carrying a plasmid expressing enzymes that catalyze the reactions A, B, E, and F

By the same method as in Comparative Example 8, the production test of 3-hydroxyadipic acid and α-hydromuconic acid was conducted using the Ec/3HA1 produced in Reference Example 6.

By comparing the results of Example 24 and Example 16, it was found that the yields of 3-hydroxyadipic acid and α-hydromuconic acid were further increased in the E. coli mutant with defects in the genes encoding pyruvate kinase and the phosphotransferase system enzyme and with enhanced activity of an enzyme that catalyzes the reaction of reducing 3-oxoadipyl-CoA to 3-hydroxyadipyl-CoA. Additionally, by comparing the results of Example 24 and Comparative Example 12, it was found that the yields of acetic acid and ethanol, both of which were generated by conversion of acetyl-CoA, were also increased in the mutant with defects in the genes encoding pyruvate kinase and the phosphotransferase system enzyme.

TABLE 18

| | | | | Yield of | Yield of | |
|---|---|---|---|---|---|---|
| | | Yield of | Yield of | succinic | acetic | Yield of |
| | Strain | 3HA (%) | HMA (%) | acid (%) | acid (%) | ethanol (%) |
| Example 24 | EcΔPPG/3HA1 | 15.4 | 0.0439 | 60.2 | 51.3 | 58.0 |
| Comparative | Ec/3HA1 | 1.32 | 0.0171 | 12.6 | 36.7 | 40.7 |
| Example 12 | | | | | | |

1. A genetically modified microorganism in which a nucleic acid encoding any one of the polypeptides described in (a) to (c) below is introduced or the expression of the polypeptide is enhanced and the function of pyruvate kinase is impaired:
(a) a polypeptide composed of an amino acid sequence represented by any one of SEQ ID NOs: 1 to 7;
(b) a polypeptide composed of the same amino acid sequence as that represented by any one of SEQ ID NOs: 1 to 7, except that one or several amino acids are substituted, deleted, inserted, andlor added, and having an enzymatic activity that catalyzes a reaction to reduce 3-oxoadipyl-CoA to 3-hydroxyadipyl-CoA;
(c) a polypeptide composed of an amino acid sequence with a sequence identity of not less than 70% to the sequence represented by any one of SEQ ID NOs: 1 to 7 and having an enzymatic activity that catalyzes a reaction to reduce 3-oxoadipyl-CoA to 3-hydroxyadipyl-CoA.

2. The genetically modified microorganism according to claim 1, wherein a polypeptide selected from the above (b) and (c) contains a region composed of an amino acid sequence represented by SEQ ID NO: 173.

3. The genetically modified microorganism according to claim 2, wherein the amino acid sequence represented by SEQ ID NO: 173 contains a phenylalanine or leucine residue at the 13th amino acid position from the N terminus, a leucine or glutamine residue at the 15th amino acid position from the N terminus, a lysine or asparagine residue at the 16th amino acid position from the N terminus, a glycine or serine residue at the 17th amino acid position from the N terminus, a proline or arginine residue at the 19th amino acid position from the N terminus, and a leucine, methionine, or valine residue at the 21st amino acid position from the N terminus.

4. The genetically modified microorganism according to any one of claim 1, which is a genetically modified microorganism belonging to a genus selected from the group consisting of Escherichia, Serratia, Hafnia, and Pseudomonas.

5. The genetically modified microorganism according to claim 1, which has an ability to generate 3-oxoadipyl-CoA and coenzyme A from acetyl-CoA and succinyl-CoA and an ability to generate 3-hydroxyadipic acid from 3-hydroxyadipyl-CoA.

6. The genetically modified microorganism according to claim 1, which has an ability to generate 3-oxoadipyl-CoA and coenzyme A from acetyl-CoA and succinyl-CoA, an ability to generate 2,3-dehydroadipyl-CoA from 3-hydroxyadipyl-CoA, and an ability to generate α-hydromuconic acid from 2,3-dehydroadipyl-CoA.

7. The genetically modified microorganism according to claim 1, which has an ability to generate 3-oxoadipyl-CoA and coenzyme A from acetyl-CoA and succinyl-CoA, an ability to uenerate 2,3-dehydroadipyl-CoA from 3-hydroxyadipyl-CoA, an ability to generate adipyl-CoA from 2,3-dehydroadipyl-CoA, and an ability to generate adipic acid from adipyl-CoA.

8. The genetically modified microorganism according to claim 1, wherein the function of a phosphotransferase system enzyme is further impaired.

9. A method of producing 3-hydroxyadipic acid, comprising culturing the uenetically modified microorganism according to claim 1 in a culture medium containing a carbon source as a raw material for fermentation.

10. A method of producing α-hydromuconic acid, comprising culturing the genetically modified microorganism according to claim 1 in a culture medium containing a carbon source as a raw material for fermentation.

11. A method of producing adipic acid, comprising culturing the genetically modified microorganism according to claim 1 in a culture medium containing a carbon source as a raw material for fermentation.

12. A method of producing one or more substances selected from the group consisting of 3-hydroxyadipic acid, α-hydromuconic acid, and adipic acid, comprising culturing a uenetically modified microorganism in a culture medium containing a carbon source as a raw material for fermentation, Wherein a nucleic acid encoding a polypeptide encoded by the 3-hydroxybutyryl-CoA dehydrogenase gene of a microorganism of the genus Serratia , which forms a gene cluster with 5-aminolevulinic acid synthase gene in the microorganism, is introduced or the expression of the polypeptide is enhanced and the function of pyruvate kinase is impaired in the genetically modified microorganism.

13. The method according to claim 12, wherein the genetically modified microorganism is a microorganism in which the function of a phosphotransferase system enzyme is further impaired.
